(19) United States
(12) Patent Application Publication
(10) Pub. No.: US 2021/0052712 A1
(43) Pub. Date: Feb. 25, 2021

(54) HETEROLOGOUS COMBINATION PRIME:BOOST THERAPY AND METHODS OF TREATMENT

(71) Applicant: CHILDREN'S HOSPITAL OF EASTERN ONTARIO RESEARCH INSTITUTE INC., Ottawa (CA)

(72) Inventor: David F. STOJDL, Ottawa (CA)

(73) Assignee: CHILDREN'S HOSPITAL OF EASTERN ONTARIO RESEARCH INSTITUTE INC., Ottawa, ON (CA)

(21) Appl. No.: 17/045,753

(22) PCT Filed: Apr. 9, 2019
(86) PCT No.: PCT/CA2019/050433

§ 371 Date: Oct. 6, 2020

(51) Int. Cl. A61K 39/00; C07K 14/005; C07K 14/47; A61K 39/12; A61K 35/17; A61K 9/00; A61P 35/00; C12N 7/00; A61K 39/205

(52) U.S. Cl. CPC A61K 39/001186; A61K 2039/5256; C07K 14/47; A61K 39/12; A61K 35/17; A61K 9/0019; A61P 35/00; C12N 7/00; A61K 39/205; A61K 9/0085; A61K 2039/545; A61K 2039/585; A61K 2039/54; A61K 2039/55516; A61K 2039/572; C12N 2760/20043; C12N 2760/20071; C12N 2760/20034; C12N 2710/20022; C12N 2710/20071; C07K 14/005

(57) ABSTRACT

The present disclosure provides a Farmington virus formulated to induce an immune response in a mammal against a tumour associated antigen. The Farmington virus may express an antigenic protein that includes an epitope from the tumour associated antigen. The Farmington virus may be formulated in a composition where the virus is separate from an antigenic protein that includes an epitope from the tumour associated antigen. The present disclosure also provides a prime:boost therapy for use in inducing an immune response in a mammal. The boost includes a Farmington virus, or a composition that includes a Farmington virus.

### FIELD

The present disclosure relates to Farmington (FMT) virus and its use in cancer treatment.

### BACKGROUND

Pathogens and disease cells comprise antigens that can be detected and targeted by the immune system, thus providing a basis for immune-based therapies, including immunogenic vaccines and immunotherapies. In the context of cancer treatment, for example, immunotherapy is predicated on the fact that cancer cells often have molecules on their cell surfaces that can be recognized and targeted.

Viruses have also been employed in cancer therapy, in part for their ability to directly kill disease cells. For example, oncolytic viruses (OVs) specifically infect, replicate in and kill malignant cells, leaving normal tissues unaffected. Several OVs have reached advanced stages of clinical evaluation for the treatment of various neoplasms. In addition to the vesicular stomatitis virus (VSV), the non-VSV Maraba virus has shown oncotropism in vitro. Maraba virus, termed “Maraba MG1” or “MG1”, has been engineered to have improved tumour selectivity and reduced virulence in normal cells, relative to wild-type Maraba. MG1 is a double mutant strain containing both G protein (Q242R) and M protein (L123W) mutations. In vivo MG1, has potent anti-tumour activity in xenograft and syngeneic tumour models in mice that is superior to the therapeutic efficacy observed with the attenuated VSV, VSVΔM51 oncolytic viruses that preceded MG1 (WO 2011/070440).

Various strategies have been developed to improve OV-induced anti-tumour immunity. The strategies take advantage of both the inherent oncolytic activity of the virus, and the ability to use the virus as a vehicle to generate immunity to tumour associated antigens. One such strategy, defined as an “oncolytic vaccine”, involves the modification of an oncolytic virus so that it contains nucleic acid sequences that expresses one or more tumour antigen(s) in vivo. It has been demonstrated that VSV can also be used as a cancer vaccine vector. Human Dopachrome Tautomerase (hDCT) is an antigen present on melanoma cancers. When administered in a heterologous prime:boost setting in a murine melanoma model, a VSV expressing hDCT not only induced an increased tumour-specific immunity to DCT but also a concomitant reduction in antiviral adaptive immunity. As a result, an increase of both median and long term survival were seen in the model system.

Farmington virus is a member of the Rhabdoviridae family of single-stranded negative sense RNA viruses and has been previously demonstrated to have oncolytic properties. It was first isolated from a wild bird during an outbreak of epizootic eastern equine encephalitis.

There remains a need for improved oncolytic vaccine vectors and treatment regimens that deliver improved immunogenicity to target cancer antigens while retaining, or even improving the overall oncolytic efficacy of the treatment.

### SUMMARY

The following disclosure is intended to exemplify, not limit, the scope of the invention.

The goal of the invention is to develop a new, improved oncolytic virus capable of being modified into an oncolytic vaccine, e.g., to both function at a therapeutic oncolytic level while eliciting a therapeutic immune response to a tumour associated antigen in a mammal with a cancer expressing the same tumour associated antigen. The oncolytic virus of the invention is capable of being used as the boost component of a heterologous prime:boost therapy. When administered as, for example, using the methods described here the resulting prime:boost therapy provides improved efficacy to when substituted into or added to one or more previously disclosed prime:boost combination therapies. See, e.g., International Application Nos. WO 2010/105347, WO 2014/127478, and WO 2017/195032, the entire contents of each of which are herein incorporated by reference.

In one aspect, the present disclosure provides a Farmington virus formulated to induce an immune response in a mammal against a tumour associated antigen. In some embodiments, the Farmington virus is capable of expressing an antigenic protein that includes an epitope from the tumour associated antigen. In some embodiments, the Farmington virus is formulated in a composition where the virus is separate from an antigenic protein that includes at least one epitope from the tumour associated antigen.

In another aspect, the present disclosure provides a heterologous combination prime:boost therapy for use in inducing an immune response in a mammal. The prime is formulated to generate an immunity in the mammal to a tumour associated antigen. The boost includes a Farmington virus, and is formulated to induce the immune response in the mammal against the tumour associated antigen. Aside from the immunological responses to the tumour associated antigen, the prime and the boost are immunologically distinct.

In yet another aspect, the present disclosure provides a composition comprising a boost for use in inducing an immune response to a tumour associated antigen in a mammalian subject having a pre-existing immunity to the tumour associated antigen. The boost includes a Farmington virus, and is formulated to induce the immune response in the mammal against the tumour associated antigen. The pre-existing immunity may be generated by a prime from a combination prime:boost treatment. In such an example, the immune response generated by the boost is based on the same tumour associated antigen as the immune response generated by the prime that is used in the prime:boost treatment. Aside from the immunological response, the boost is immunologically distinct from the prime.

In still another aspect, the present disclosure provides a Farmington virus formulated to induce an immune response in a mammal against a tumour associated antigen. The Farmington virus is for use as the boost of a pre-existing immunity to the tumour associated antigen. The pre-existing immunity may be generated by the prime of a combination prime:boost therapy. The prime of the combination prime:boost therapy is formulated to generate an immunity in the mammal to the tumour associated antigen and, aside from the immunological responses to the tumour associated antigen, the boost is immunologically distinct from the prime.

In one aspect, the present disclosure provides a Farmington virus comprising a nucleic acid that is capable of expressing a tumour associated antigen or an epitope thereof. In some embodiments, the genomic backbone of the Farmington virus encodes a protein having at least 90% sequence identity with any one of SEQ ID NOs 3-7. In some embodiments, the genomic backbone of the Farmington virus encodes a protein having at least 95% sequence identity with any one of SEQ ID NOs 3-7.

In some embodiments, the tumour associated antigen (“TAA”) is a foreign antigen. For example, the foreign antigen may comprise may comprise an antigenic portion, portions, or derivatives, or the entire tumour-associated foreign antigen. Exemplary foreign TAA's used in the methods of the invention may be or be derived from a fragment or fragments of known TAA's. Foreign TAA's include E6 protein from Human Papilloma Virus (“HPV”); E7 protein from HPV; E6/E7 fusion protein; human CMV antigen, pp65; murine CMV antigen, m38; and others.

In some embodiments, the tumour associated antigen (“TAA”) is a self antigen. For example, the self antigen may comprise an antigenic portion, portions, or derivatives, or the entire tumour-associated self antigen. Exemplary self TAA's used in the methods of the invention may be or be derived from a fragment or fragments of known TAA's. Self TAA's include human dopachrome tautomerase (hDCT) antigen; melanoma-associated antigen (“MAGEA3”); human Six-Transmembrane Epithelial Antigen of the prostate protein (“huSTEAP”); human Cancer Testis Antigen 1 (“NYESO1”); and others.

In some embodiments, the tumour associated antigen is a neoepitope.

In some embodiments, the Farmington virus induces an immune response against the tumour associated antigen in a mammal to whom the Farmington virus is administered. In some embodiments, the mammal has been previously administered a prime that is immunologically distinct from the Farmington virus.

In some embodiments, the prime is, for example,

(a) a virus comprising a nucleic acid that is capable of expressing the tumour associated antigen or an epitope thereof;

(b) T-cells specific for the tumour associated antigen; or

(c) a peptide of the tumour associated antigen.

In some embodiments, the Farmington virus further encodes a cell death protein.

In one aspect, the present disclosure provides a composition comprising a Farmington virus comprising a nucleic acid that is capable of expressing a tumour associated antigen or an epitope thereof, the composition being formulated to induce an immune response in a mammal against the tumour associated antigen.

In one aspect, the present disclosure provides a composition comprising a Farmington virus and an antigenic protein that includes an epitope from a tumour associated antigen, wherein the Farmington virus is separate from the antigenic protein, the composition being formulated to induce an immune response in a mammal against the tumour associated antigen.

In one aspect, the present disclosure provides a heterologous combination prime:boost therapy for use in inducing an immune response in a mammal, wherein the prime is formulated to generate an immunity in the mammal to a tumour associated antigen, and the boost comprises: a Farmington virus comprising a nucleic acid that is capable of expressing a tumour associated antigen or an epitope thereof and is formulated to induce the immune response in the mammal against the tumour associated antigen.

In one aspect, the present disclosure provides a method of enhancing an immune response in a mammal having a cancer, the method comprising a step of: administering to the mammal a composition comprising a Farmington virus comprising a nucleic acid that is capable of expressing a tumour associated antigen or an epitope thereof,

wherein the mammal has been administered a prime that is directed to the tumour associated antigen or an epitope thereof; and wherein the prime is immunologically distinct from the Farmington virus.

In some embodiments, the mammal has a tumour that expresses the tumour associated antigen.

In some embodiments, the cancer is brain cancer. For example, the brain cancer may be glioblastoma.

In some embodiments, the cancer is colon cancer.

In some embodiments, the Farmington virus is capable of expressing an epitope of the tumour associated antigen.

In some embodiments, the prime is directed to an epitope of the tumour associated antigen.

In some embodiments, the prime is directed to the same epitope of the tumour associated antigen as the epitope encoded by the Farmington virus.

In some embodiments, the prime comprises: (a) a virus comprising a nucleic acid that is capable of expressing the tumour associated antigen or an epitope thereof; (b) T-cells specific for the tumour associated antigen; or (c) a peptide of the tumour associated antigen.

In some embodiments, the prime comprises a virus comprising a nucleic acid that is capable of expressing the tumour associated antigen or an epitope thereof. For example, the prime may comprise a single-stranded RNA virus, such as a positive-strand RNA virus (e.g., lentivirus) or a negative-strand RNA virus. In some embodiments, the prime comprises a double-stranded DNA virus. For example, the double-stranded DNA virus may be an adenovirus (e.g., an Ad5 virus).

In some embodiments, the prime comprises T-cells specific for the tumour associated antigen.

In some embodiments, the prime comprises a peptide of the tumour associated antigen. In some such embodiments, the prime further comprises an adjuvant.

In some embodiments, the mammal is administered the composition at least 9 days after the mammal was administered the prime. In some embodiments, the mammal is administered the composition no more than 14 days after the mammal was administered the prime.

In some embodiments, provided methods further comprise a second step of administering to the mammal a composition comprising a Farmington virus comprising a nucleic acid that is capable of expressing a tumour associated antigen or an epitope thereof. In some embodiments, the second step of administering is performed at least 50, at least 75, at least 100, or at least 120 days after the first step of administering.

In some embodiments, provided methods further comprise a third step of administering to the mammal a composition comprising a Farmington virus comprising a nucleic acid that is capable of expressing a tumour associated antigen or an epitope thereof. In some embodiments, the third step of administering is performed at least 50, at least 75, at least 100, or at least 120 days after the second step of administering.

In some embodiments, at least one step of administering is performed by a systemic route of administration.

In some embodiments, at least one step of administering is performed by a non-systemic route of administration.

In various embodiments, at least one step of administering is performed by injection directly into a tumour of the mammal, intracranially, intravenously, or both intravenously and intracranially.

In some embodiments, the frequency of T cells specific for the tumour associated antigen is increased after the step of administering. In some embodiments, the T cells comprise CD8 T cells.

In some embodiments, the mammal's survival is extended compared to that of a control mammal who is not administered the composition. In some embodiments, the control mammal is administered a prime directed to the tumour associated antigen, wherein the prime is immunologically distinct from the composition.

In some embodiments, the frequency of T cells specific for the Farmington virus increases by no more than 3% after the step of administering. In some embodiments, the frequency of CD8 T cells specific for the Farmington virus increases by no more than 3% after the step of administering.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIGS. 1A-1E: Engineered Farmington (FMT) virus is a versatile cancer vaccine platform. FMT virus engineered to express m38 antigen can boost immune responses when paired with 3 different prime methods: engineered AdV-m38, ACT of m38-specific CD8 T cells or m38 peptide with adjuvant, as demonstrated by frequencies and numbers of IFNγ-secreting CD8 T cells (FIG. 1A) and IFNγ and TNF-secreting CD8 T cells (FIG. 1B) after ex-vivo peptide stimulation of PBMCs isolated from vaccinated mice 5-6 days after boost. Moreover, FMT virus can boost immune responses directed to different classes of antigens: self-antigens (e.g., DCT (FIG. 1C)); foreign antigens (e.g., m38 (FIG. 1D)); and neo-epitopes (e.g., mutated Adpgk and Reps1 (FIG. 1E)). The graphs show mean and SEM. Data was analysed with 1-way ANOVA Dunn's Multiple Comparison Test (FIGS. 1A, 1B), 1-way ANOVA Dunn's Multiple Comparison Test (FIG. 1C), Mann Whitney test (FIG. 1D), and 2-way ANOVA Bonferroni Multiple Comparison Test (FIG. 1E). AdV—adenovirus, ACT—adoptive cell trasfer, P values: *−p<0.05, **−P<0.01, ***−P<0.001.

FIGS. 2A-I: FMT-based vaccination induces long-lasting immune responses. Increases in m38-specific CD8 T cells frequencies and numbers were observed following a first boost with FMT-m38 compared to PBS control and following a second boost with FMT-m38 applied 120 days after the first boost compared to PBS control and immune response just before boost (FIG. 2A). An anti-m38 immune response was sustained for over 5 months (FIG. 2A). Homologous multiple boosts were more effective when applied with longer time interval (minimum 3 months compared to 1 month) (FIGS. 2B, 2C). Higher frequencies and numbers of neo-epitope-specific CD8 T cells were detected after vaccination in mice primed with only one peptide compared to mice primed with all 3 peptides (FIGS. 2B, 2C). These immune responses lasted for over 6 months (FIGS. 2B, 2C). Data were analysed with Mann Whitney test (FIGS. 2B, 2C, 2E, and 2H) and 1-way ANOVA Dunn's Multiple Comparison Test (FIGS. 2D and 2I). ACT—adoptive cell transfer.

FIGS. 3A-3D: Anti-tumour efficacy of FMT virus-based cancer vaccine. Treatment with FMT-m38 virus in a prime+boost setting significantly extended survival of CT2A-m38 tumour-bearing mice compared with PBS and prime only controls and induced antigen-specific CD8 T cell responses in tumour-bearing mice (FIGS. 3A, 3B, and 3C). FMT-based vaccination against Adpgk and Reps1 neo-epitopes delayed tumour progression, extended survival of MC-38-tumour bearing mice and boosted antigen-specific CD8 T cells responses (FIG. 3D). Data were analysed as follows: for FIGS. 3A-3C: Log-rank (Mantel-Cox) test for survival analysis and 1-way ANOVA Dunn's Multiple Comparison Test; for FIG. 1D Log-rank (Mantel-Cox) test for survival analysis and 2-way ANOVA Bonferroni Multiple Comparison Test. AdV—adenovirus, ACT—adoptive cell trasfer. P values: *−p<0.05, **−P<0.01, ***−P<0.001, ****−P<0.0001.

FIGS. 4A-4C: Inducing TAA-specific effector CD8 T cells provides therapeutic efficacy. Treatment with anti-m38 prime and boost induced high frequencies and numbers of m38-specific CD8 T cells and extended the survival of mice bearing m38-expressing CT2A tumours, while vaccination with irrelevant antigens did not have an impact on survival (FIG. 4A). Prime+boost treatment improved the survival of tumour-bearing mice at a ACT starting dose 10³ cells (FIG. 4B). Increasing the ACT prime dose resulted in higher frequencies and numbers of antigen-specific CD8 T cells and increased cure rate; however, no further survival benefit was observed above an ACT dose of 10⁵ cells (FIG. 4B). FMT-m38 treatment administered intravenously (iv) induced highest frequencies and numbers of m38-specific CD8 T cells and had the best therapeutic efficacy compared with intracranial (ic) (intra-tumour) route and a combination of intravenous (iv) and intracranial (ic) routes (FIG. 4C). The higher amount of infectious particles detected in the spleen after FMT virus intravenous injection compared to after intracranial injection might explain this observation (FIG. 4C). All treatment strategies extended survival, but a higher cure rate was observed in groups administered by the intravenous route alone or in combination with intracranial injection compared to intracranial injection alone (FIG. 4C).

FIGS. 5A-5E: Pre-existing TAA-specific CD8 effector T cells extend survival post tumour challenge. (See Example 8.) FIGS. 5A and 5C show percentages of CD8+ IFNγ+(out of all CD8+ cells) in blood from mice 9 days before and 6 days after, respectively, tumour challenge. FIGS. 5B and 5D show amounts of m38-specific CD8⁺ T cells per mL blood from mice 9 days before and 6 days after, respectively, tumour challenge. FIG. 5E shows Kaplan-Meier survival curves of mice receiving various prime:boost treatments or PBS.

FIGS. 6A-6E: FMT-based vaccination administered intracranially promotes anti-tumour immune response within the brain tumour microenvironment. FMT-m38 injection by both intravenous (iv) and intracranial (ic) routes increased the frequency and numbers of tumour-infiltrating lymphocytes (TILs) compared to PBS control, while numbers of macrophages remained the same in each group (FIG. 6A). In the FMT-m38 intravenous treatment group, a distinct CD11b^{low} CD45+ population of macrophages was observed (FIG. 6A). The “all macrophages” population in FIG. 6A includes both the CD11b^{low} CD45+ and CD11b+CD45^{bright} macrophage populations (red gate on dot plots). FMT-m38-based vaccination reduced the frequency and numbers of CD206+ macrophages, while CD86 expression was very similar with in PBS controls (FIG. 6B). Treatment with intracranially delivered FMT-m38 increased the recruitment of both CD8 and CD4 T cells, while reduced amounts of these cells were found in tumours from mice treated with intravenously administered FMT-m38 compared to tumours from control mice (FIG. 6C). CD8^{low} T cells were gated and considered CD8 T cells, as they formed a distinct population on the dot plot (FIG. 6C), and downregulation of CD8 marker upon activation was observed in other experiments. Intracranial injection of FMT virus increased IL-7, IL-13, IL-6 and TNFa cytokines and G-CSF growth factor levels (FIG. 6D). Elevated levels of chemokines Eotaxin, CXCL5, RANTES, CXCL1 and MIP-2 were observed in tumours from mice injected intracranially with FMT virus compared to that observed in tumors from mice in the PBS control or FMT-intravenous group. Intravenous injection resulted in diminished levels of CXCL5, MIG, RANTES and CXCL1 compared to levels in the PBS control or FMT-intracranial group (FIG. 6E).

Graphs show mean and SEM and representative dot plots from each treatment group. All data in FIGS. 6A-6C were analysed with 2 way ANOVA Bonferroni multiple comparison test, except CD206+ cell numbers, which were analysed with Kruskal-Wallis and Dunn's multiple comparison test. All data in FIGS. 6D and 6E were analysed with Kruskal-Wallis and Dunn's multiple comparison test. P values: *−p<0.05, **−P<0.01, ***−P<0.001, ****−P<0.0001.

FIG. 7A-7C: Ex vivo expansion of antigen-specific central memory CD8 T cells. Splenocytes were extracted from Maxim38 mice and cultured for 6 days in supplemented RPMI medium in the presence of m38 peptide. On the day of harvest, cells were phenotyped by flow cytometry. The majority of cells were CD8-positive (FIG. 7A). Within the CD8+ population, 40-60% of cells were of memory CD127+CD62L+ phenotype (FIG. 7B). Most of memory T cells expressed CD27, none expressed KLRG1 and the expression of CCR7 varied between different cellular products, but in most cases was low (FIG. 7C).

FIG. 8. CD8 T cell response to FMT viral backbone. CD8 T cell response against a dominant epitope of FMT virus was assessed by peptide stimulation and intracelluar cytokine staining (ICS) assay 5-6 days after FMT-m38 boost. The frequencies of FMT-specific CD8 T cells ranged from 0-3% and were significantly higher compared to PBS control only in a group primed with ACT-m38. 1-way ANOVA Dunn's Multiple Comparison Test. AdV—adenovirus, ACT—adoptive cell trasfer, P values: * −p<0.05, **−P<0.01, ***−P<0.001.

FIGS. 9A and 9B. CT2A-m38 brain tumour model characteristics. MRI imaging of brains in mice injected with wild type CT2A cells (left panels) vs. those of mice injected with CT2A-m38 cells (FIG. 9A). Expression of a major histocompatibility complex class I (MHC I) allele that presents the m38 epitope in tumour cells extracted from mice 21 days after intracranial implantation of CT2A-m38 cells (FIG. 9B).

FIG. 10. Immune response at the day of brain tumour collection. Blood was collected from CT2A-m38 tumour-bearing mice 6 days after FMT-m38 is or iv injection. FMT-m38 boost expanded the frequencies and numbers of m38-specific cells.

FIGS. 11A-11D. Gating strategy for phenotyping of tumour-infiltrating immune cells. The debris and dead cells were excluded on the FSC vs SSC plot, then singlets were gated on the FSC-A vs SSC-A plot, and remaining dead cells were excluded by Viability dye stain (FIG. 11A). Immune cells were gated based on the expression of CD45 (FIG. 11B). Next, within the CD45+ population, we distinguished microglia (defined as the CD11 b+CD45^{low} population), all macrophages (red gate) (defined as CD11b+CD45^{bright} cells), and lymphocytes (defined as CD11 b-CD45+ cells) (FIG. 11C). Expression of the NK cell marker NKp46 within all CD45+ cells was also examined; however, this population was less than 0.5% of all immune cells (data not shown). The “all macrophages” population was further divided into CD11b+CD45^{bright} and CD11 b^{low}CD45+ populations (FIG. 11C). Both macrophage and microglia populations may also contain dendritic cells and granulocytes. Within the CD11b-CD45+ lymphocyte population, T cells were gated as CD3+ cells (FIG. 11D). Macrophages and T cells were further examined for the expression of other markers as indicated in FIGS. 5A-E. FSC-A—Forward Scatter-Area, FSC-H—Forward Scatter-Height, SSC—Side Scatter-Area.

### DETAILED DESCRIPTION

Generally, the present disclosure provides Farmington virus and its use as, or in, an immunostimulatory composition. The Farmington virus may be used as a boost of a pre-existing immunity to a tumour associated antigen. The boost may be a component in a heterologous combination prime:boost treatment, where the prime generates the pre-existing immunity. In heterologous prime:boost treatments, the prime and the boost are immunologically distinct.

In the context of the present disclosure, the expression “immunologically distinct” should be understood to mean that at least two agents or compositions (e.g., the prime and the boost) do not produce antisera that cross react with one another. The use of a prime and a boost that are immunologically distinct permits an effective prime/boost response to the tumour associated antigen that is commonly targeted by the prime and the boost.

In the context of the present disclosure, a “combination prime:boost therapy” should be understood to refer to therapies for which (1) the prime and (2) the boost are to be administered as a prime:boost treatment. A “therapy” should be understood to refer to physical components, while a “treatment” should be understood to refer to the method associated with administration of the therapeutic components. The prime and boost need not be physically provided or packaged together, since the prime is to be administered first and the boost is to be administered only after an immunological response has been generated in the mammal. In some examples, the combination may be provided to a medical institute, such as a hospital or doctor's office, in the form of a package (or plurality of packages) of the prime, and a separate package (or plurality of packages) of the boost. The packages may be provided at different times. In other examples, the combination may be provided to a medical institute, such as a hospital or doctor's office, in the form of a package that includes both the prime and the boost. In yet other examples, the prime may be generated by a medical institute, such as through isolation of T-cells from the mammal for adoptive cell transfer, and the boost may be provided at a different time.

In the context of the present disclosure, the expression “tumour associated antigen,” “self tumour associated antigen,” is meant to refer to any immunogen that is that is associated with tumour cells, and that is either absent from or less abundant in healthy cells or corresponding healthy cells (depending on the application and requirements). For instance, the tumour associated antigen may be unique, in the context of the organism, to the tumour cells. Examples of such antigens include but are not limited to human dopachrome tautomerase (hDCT) antigen; melanoma-associated antigen (“MAGEA3”); human Six-Transmembrane Epithelial Antigen of the prostate protein (“huSTEAP”); human Cancer Testis Antigen 1 (“NYESO1”); and others.

In the context of the present disclosure, the expression “foreign antigen” or “non-self antigen” refers to an antigen that originates outside the body of an organism, e.g., antigens from viruses or microorganisms, foods, cells and substances from other organisms, etc. Examples of such antigens include but are not limited to E6 protein from Human Papilloma Virus (“HPV”); E7 protein from HPV; E6/E7 fusion protein; E6/E7 fusion protein; human CMV antigen, pp65; murine CMV antigen, m38; and others.

In the context of the present disclosure, the term “neo-antigen” refers to newly formed antigens that have not previously been recognized by the immune system and that arise from genetic aberrations within a tumor.

In the context of the present disclosure, the expression “self antigen” refers to an antigen that originates within the body of an organism.

The boost is formulated to generate an immune response in the mammal to a tumour associated antigen. The boost may be, for example: a Farmington virus that expresses an antigenic protein; a composition that includes a Farmington virus and a separate antigenic protein; or a cell infected with a Farmington virus that expresses an antigenic protein.

The full-length genomic sequence for wild type Farmington virus has been determined. The sequence of the complementary DNA (cDNA) polynucleotide produced by Farmington virus is shown in SEQ ID NO: 1 (SEQ ID NO: 1 of WO2012167382). The disclosure of WO2012167382 is incorporated herein by reference. The RNA polynucleotide sequence of Farmington virus is shown in SEQ ID NO: 2 (SEQ ID NO: 2 of WO2012167382). Five putative open reading frames were identified in the genomic sequence. Additional ORFs may be present in the virus that have not yet been identified. The sequences of the corresponding proteins are shown in SEQ ID NOs: 3, 4, 5, 6, and 7 (SEQ ID NOs: 3, 4, 5, 6 and 7 of WO2012167382).

Table 1 provide a description of SEQ ID NOs: 1-7.

TABLE 1
Description of Sequences

| | | |
|---|---|---|
| SEQ ID NO: 1 | Farmington | cDNA produced by the FMT |
| | rhabdovirus- | rhabdovirus |
| | DNA | |
| SEQ ID NO: 2 | Farmington | |
| | rhabdovirus- | |
| | RNA | |
| SEQ ID NO: 3 | Farmington | The promoter is at position 134 to |
| | rhabodvirus | 149 and the encoding sequence is at |
| | ORF1 | positions 206 to 1444 of SEQ ID |
| | | NO: 1. |
| SEQ ID NO: 4 | Farmington | The promoter is at positions 1562 to |
| | rhabodvirus | 1578 and the encoding sequence is |
| | ORF2 | at positions 1640 to 2590 of SEQ ID |
| | | NO: 1. |
| SEQ ID NO: 5 | Farmington | The promoter is at positions 2799 to |
| | rhabodvirus | 2813 and the encoding sequence is |
| | ORF3 | at positions 2894 to 3340 of SEQ ID |
| | | NO: 1. |
| SEQ ID NO: 6 | Farmington | The promoter is at positions 3457 to |
| | rhabodvirus | 3469 and the encoding sequence is |
| | ORF4 | at positions 3603 to 5717 of SEQ ID |
| | | NO: 1. |
| SEQ ID NO: 7 | Farmington | The promoter is at positions 5766 to |
| | rhabodvirus | 5780 and the encoding sequence is |
| | ORF5 | at positions 5832 to 12221 of SEQ ID |
| | | NO: 1. |

The encoding DNA sequences are shown in SEQ ID Nos: 8, 9, 10, 11, and 12 respectively (SEQ ID NOs: 8, 9, 10, 11 and 12, respectively, of WO2015154197). (The disclosures of WO 2012/167382 and WO2 015/154197 are incorporated herein by reference.)

In the context of the present disclosure, the expression “a Farmington virus” should be understood to refer to any virus whose genomic backbone encodes:
a protein that is at least 90% identical, and more preferably at least 95% identical, to the protein of SEQ ID NO: 3 (SEQ ID NO: 3 of WO2012167382);
a protein that is at least 90% identical, and more preferably at least 95% identical, to the protein of SEQ ID NO: 4 (SEQ ID NO: 4 of WO2012167382);
a protein that is at least 90% identical, and more preferably at least 95% identical, to the protein of SEQ ID NO: 5 (SEQ ID NO: 5 of WO2012167382);
a protein that is at least 90% identical, and more preferably at least 95% identical, to the protein of SEQ ID NO: 6 (SEQ ID NO: 6 of WO2012167382); and
a protein that is at least 90% identical, and more preferably at least 95% identical, to the protein of SEQ ID NO: 7 (SEQ ID NO: 7 of WO2012167382).

A Farmington virus according to the present disclosure that expresses an antigenic protein (e.g., a tumour associated antigen or an epitope thereof) may have the nucleic acid sequence encoding the antigenic protein inserted anywhere in the genomic backbone that does not interfere with the production of the viral gene products. For example: the sequence encoding the antigenic protein may be located between the N and the P genes, between the P and the M genes, or between the G and the L genes.

A Farmington virus according to the present disclosure that expresses an antigenic protein may additionally include a nucleic acid sequence that encodes a protein implicated in cell death (“cell death protein”), or a variant thereof. Examples of cell death proteins include, but are not limited to: Apoptin; Bcl-2-associated death promoter (BAD); BCL2-antagonist/killer 1 (BAK1); BCL2-associated X (BAX); p15 BH3 interacting-domain death agonist, transcript variant 2 (BIDv2); B-cell lymphoma 2 interacting mediator of cell death (BIM); Carbamoyl-phosphate synthetase 2, aspartate transcarbamylase, and dihydroorotase (CAD); caspase 2 (CASP2); caspace 3 (CASP3); caspace 8 (CASP8); CCAAT-enhancer-binding protein homologous protein (CHOP); DNA fragmentation factor subunit alpha (DFFA); Granzyme B; activated c-Jun N-terminal kinase (JNK); Phorbol-12-myristate-13-acetate-induced protein 1 (PMAPI 1, also referred to as NOXA); p53 upregulated modulator of apoptosis beta (PUMA beta); p53 upregulated modulator of apoptosis gamma (PUMA gamma); p53-induced death domain protein (PIDD); recombinant ADAM15 disintegrin domain (RAIDD); ubiquitin conjugated Second Mitochondrial-derived Activator of Caspases (SMAC); autophagy related 12 (ATG12); autophagy related 3 (ATG3); Beclin-1 (BECN1); solute carrier family 25 member 4 (SLC25A4); Receptor-interacting serine/threonine-protein kinase 1 (RIPK1); Receptor-interacting serine/threonine-protein kinase 3 (RIPK3); short form of Phosphoglycerate mutase family member 5 (PGAM5S); mixed lineage kinase domain-like (MLKL); Cathepsin D; Maraba M; and any variant thereof.

Specific examples of such an additional protein are: mixed lineage kinase domain-like (MLKL), caspase 2 (CASP2), p15 BH3 interacting-domain death agonist, transcript variant 2 (BIDv2), and Bcl-2-associated death promoter (BAD).

Farmington viruses that encode cell death proteins, or variants thereof, are discussed in WO2015154197, the disclosure of which is incorporated herein by reference. Specific examples of the MLKL, CASP2, BIDv2, and BAD proteins have the sequences shown in SEQ ID NOs: 13, 15, 17 and 19, respectively, of WO2015154197.

The prime and the boost may include different antigenic proteins, so long as the antigenic proteins are based on the same tumour associated antigen. This should be understood to mean that the antigenic protein of the prime and the antigenic protein of the boost are design or selected, such that they each comprise sequences eliciting an immune reaction to the same tumour associated antigen. It will be appreciated that the antigenic protein of the prime and the antigenic protein of the boost need not be exactly the same in order to accomplish this. For instance, they may be peptides comprising sequences that partially overlap, with the overlapping segment comprising a sequence corresponding to the tumour associated antigen, or a sequence designed to elicit an immune reaction to the tumour associated antigen, thereby allowing an effective prime and boost to the same antigen to be achieved. However, in some embodiments, the antigenic protein of the prime and the antigenic protein of the boost are the same.

The prime, formulated to generate an immunity in the mammal to a tumour associated antigen, may be any combination of components that potentiates the immune response to the tumour associated antigen. For example, the prime may be, or may include: a virus that expresses an antigenic protein; a mixture of a virus and an antigenic protein; a pharmacological agent and an antigenic protein; an immunological agent and an antigenic protein (e.g., an adjuvant and a peptide); adoptive cell transfer; or any combination thereof. In the context of the present disclosure, the subject may have prior exposure to certain antigens unrelated to the present therapy. Any immune response to such prior exposure is not considered a “prime” for the purpose of the presently disclosed methods and compositions.

In some embodiments, the prime comprises

(a) a virus comprising a nucleic acid that is capable of expressing the tumour associated antigen or an epitope thereof;

(b) T-cells specific for the tumour associated antigen; or

(c) a peptide of the tumour associated antigen.

In some embodiments, the prime comprises an oncolytic virus.

In some embodiments, the prime comprises a virus comprising a nucleic acid that is capable of expressing the tumour associated antigen or an epitope thereof.

In some embodiments, the prime comprises a single-stranded RNA virus.

The single-stranded RNA virus may be a positive-sense single stranded RNA virus (e.g., a lentivirus) or a negative-sense single stranded RNA virus.

In some embodiments, the prime comprises a double-stranded DNA virus.

For example, the virus may be an adenovirus, e.g., an Ad5 virus.

In some embodiments, the prime comprises T-cells specific for the tumour associated antigen. For example, the prime may comprise T-cells of the memory phenotype, e.g., CD8+ memory cells (e.g., CD8+CD127+CD62L+ cells).

In some embodiments, the prime comprises a peptide, e.g., an epitope of a tumour associated antigen. In some such embodiments, the prime further comprises an adjuvant.

More specific examples of primes contemplated by the authors include: an adenovirus that expresses an antigenic protein; a lentivirus that expresses an antigenic protein; Listeria monocytogenes (LM) that expresses an antigenic protein; an oncolytic virus that expresses an antigenic protein; an adenovirus and an antigenic protein where the antigenic protein is not encoded by the adenovirus; an oncolytic virus and an antigenic protein where the antigenic protein is not encoded by the oncolytic virus; a mixture of poly I:C and an antigenic protein; CD8 memory T-cells specific to an antigenic protein; a mixture of poly I:C, anti CD40 antibody, and an antigenic protein; and a nanoparticle adjuvant with an immunostimulatory RNA or DNA, or with an antigenic protein.

The tumour associated antigen may be, for example, an antigen in: Melanoma Antigen, family A,3 (MAGEA3); human Papilloma Virus E6 protein (HPV E6); human Papilloma Virus E7 protein (HPV E7); human Six-Transmembrane Epithelial Antigen of the Prostate protein (huSTEAP); Cancer Testis Antigen 1 (NYESO1); Brachyury protein; Prostatic Acid Phosphatase; Mesothelin; CMV pp65; CMV IE1; EGFRvIII; IL13R alpha2; Her2/neu; CD70; CD133; BCA; FAP; Mesothelin; KRAS; p53; CHI; CSP; FABP7; NLGN4X; PTP; H3F3A K27M; G34R/V; or any combination thereof. In some embodiments, the tumor associated antigen is a foreign antigen. In some embodiments, the tumor associated antigen is a self antigen. In some embodiments, the tumour associated antigen is a neo-antigen that results from a tumour-specific mutation of a wild-type self-protein.

The protein sequence of full length, wild type, human MAGEA3 is shown in SEQ ID NO: 13 (SEQ ID NO: 1 of WO/2014/127478). The protein sequence of a variant of full length, wild type, human MAGEA3 is shown in SEQ ID NO; 14 (SEQ ID NO: 4 of WO/2014/127478). The protein sequences of HPV16 E6, HPV18 E6, HPV16 E7 and HPV18 E7 are shown in SEQ ID NOs: 15-18 (SEQ ID Nos: 9-12 of WO/2017/195032). The protein sequence of a huSTEAP protein is shown in SEQ ID NO: 19 (SEQ ID NO: 13 of WO/2017/195032). The protein sequence of NYESO1 is shown in SEQ ID NO: 20 (SEQ ID NO: 13 of WO/2014/127478). The protein sequence of human Brachyury protein is disclosed in the Uniprot database under identifier 015178-1 (www.uniprot.org/uniprot/015178) (SEQ ID NO: 21). The protein sequence of secreted human prostatic acid phosphatase is disclosed in the Uniprot database under identifier P15309-1 (www.uniprot.org/uniprot/P15309) (SEQ ID NO: 22). The disclosure of which is incorporated herein by reference. Variants of these specific sequences may be used as antigenic proteins for the prime and/or the boost of the present disclosure so long as the variant protein includes at least one tumour associated epitope of the reference protein, and the amino acid sequence of the variant protein is at least 70% identical to the amino acid sequence of the reference protein.

In one aspect, the present disclosure provides a heterologous combination prime:boost therapy for use in inducing an immune response in a mammal. The prime is formulated to generate an immunity in the mammal to a tumour associated antigen. The boost includes a Farmington virus, and is formulated to induce the immune response in the mammal against the tumour associated antigen. Aside from the immune responses to the tumour associated antigen, the prime and the boost are immunologically distinct.

In some embodiments, the prime:boost therapy is formulated to generate immune responses against a plurality of antigens. It should be understood that antigenic proteins, such as MAGEA3, HPV E6, HPV E7, huSTEAP, Cancer Testis Antigen 1; Brachyury; Prostatic Acid Phosphatase; FAP; HER2; and Mesothelin have more than one antigenic epitope. Formulating the prime and the Farmington virus to include or express an antigenic protein having a plurality of antigenic epitopes may result in the mammal generating immune responses against more than one of the antigenic epitopes.

In one specific example, the prime and the Farmington virus are both formulated to induce an immune response against at least one antigen in the E6 and E7 transforming proteins of the HPV16 and HPV18 serotypes. This may be accomplished by having the Farmington virus express a fusion protein that includes HPV16 E6, HPV18 E6, HPV16 E7 and HPV18 E7 protein domains. The four protein domains are linked by proteasomally degradable linkers that result in the separate HPV16 E6, HPV18 E6, HPV16 E7 and HPV18 E7 proteins once the fusion protein is in the proteasome. Exemplary fusion proteins are discussed in WO/2014/127478 and WO/2017/195032, the disclosures of which are incorporated herein by reference. The prime may be formulated to induce an immune response against an antigenic protein that is different from the antigenic protein expressed by the Farmington virus. For example, the prime may be an oncolytic virus that expresses an HPV E6/E7 fusion protein where the four protein domains are linked in a different order.

In another specific example, the prime and the Farmington virus are both formulated to induce an immune response against at least one antigen in MAGEA3. This may be accomplished by having the Farmington virus express an antigenic protein comprising an amino acid sequence (a) that includes at least one tumour associated epitope selected from the group consisting of: EVDPIGHLY (SEQ ID NO: 23), FLWGPRALV (SEQ ID NO: 24), KVAELVHFL (SEQ ID NO: 25), TFPDLESEF (SEQ ID NO: 26), VAELVHFLL (SEQ ID NO: 27), REPVTKAEML (SEQ ID NO: 28), AELVHFLLL (SEQ ID NO: 29), WQYFFPVIF (SEQ ID NO: 30) EGDCAPEEK (SEQ ID NO: 31), KKLLTQHFVQENYLEY (SEQ ID NO: 32), VIFSKASSSLQL (SEQ ID NO: 33), VFGIELMEVDPIGHL (SEQ ID NO: 34), GDNQIMPKAGLLIIV (SEQ ID NO: 35), TSYVKVLHHMVKISG (SEQ ID NO: 36), and FLLLKYRAREPVTKAE (SEQ ID NO: 37), and (b) that is at least 70% identical to the amino acid sequence of SEQ ID NO: 13( ). The prime may be formulated to induce an immune response against an antigenic protein that is different from the antigenic protein expressed by the Farmington virus. For example, the prime may be a mixture of poly I:C and a synthetic long peptide that includes FLWGPRALV (SEQ ID NO: 24).

In yet another specific example, the prime and the Farmington virus are both formulated to induce an immune response against a neo-antigen. This may be accomplished by formulating the Farmington virus as an adjuvant to an antigenic protein that includes the neo-antigen, where the Farmington virus does not encode the antigenic protein. The prime may be formulated against the same antigenic protein or against a different antigenic protein, so long as the immunogenic sequence of the neo-antigen is conserved.

1. A prime:boost therapy according to the present disclosure may be used in the treatment of cancer. For example, in one aspect, provided are methods of enhancing an immune response in a mammal having a cancer, the method comprising a step of:

administering to the mammal a composition comprising a Farmington virus comprising a nucleic acid that is capable of expressing a tumour associated antigen or an epitope thereof,
wherein the mammal has been administered a prime is directed to the tumour associated antigen or an epitope thereof; and

wherein the prime is immunologically distinct from the Farmington virus.

In some embodiments, the mammal has brain cancer, such as glioblastoma. In some embodiments, the prime has colon cancer.

The prime and the composition comprising the Farmington virus may be administered by any of a variety of routes of administration, which may be the same or different for the prime and the composition comprising the Farmington virus. One of ordinary skill in the art reading the present specification will understand that the appropriate route of administration may depend on one or more factors, including, e.g., on the type of cancer the mammal has. In some embodiments, at least one of the prime and the composition comprising the Farmington virus is administered by a systemic route of administration. In some embodiments, at least one of the prime and the composition comprising the Farmington virus is administered by a non-systemic route of administration.

Non-limiting examples of routes of administration include intravenous, intramuscular, intraperitoneal, intranasal, intracranial, and direct injection into a tumour. For example, in the case of brain cancer, intracranial administration may be suitable. In some embodiments, the prime and/or the composition comprising the Farmington virus is administered by more than one method, e.g., both intracranially and intravenously.

In some embodiments, provided methods comprise more than one “boost” with Farmington virus, e.g., methods may further comprise a second step (and optionally a third step) of administering to the mammal a composition comprising a Farmington virus as disclosed herein. In embodiments comprising more than one “boost,” a subsequent boost may be separated by a time interval, e.g., at 50, at least 75, at least 100, or at least 120 days from the previous step of administering. In embodiments comprising at least three boosts, the time intervals between boosts may be approximately the same, or they may be different.

In some embodiments, an immune response is generated in the mammal after the step of administering the composition comprising the Farmington virus (or after each step of administering the composition). For example, the immune response can comprise an immune response specific for the tumour associated antigen (TAA), e.g., an increase in the frequency of T cells (e.g., CD8 T cells) specific for the tumour associated antigen (e.g., as determined in a sample such as a blood or serum sample from the mammal).

In some embodiments, a limited immune response, or no immune response, specific for the Farmington virus is generated in the mammal after the step of administering the composition comprising the Farmington virus (or after each step of administering the composition). For example, in some embodiments, after the step of administering the composition comprising the Farmington virus (or after each step of administering the composition), the frequency of T cells (e.g., CD8 T cells) specific for the Farmington virus is no greater than 3% (e.g., as determined in a sample such as a blood or serum sample from the mammal).

Provided prime:boost therapies may be formulated in accordance with provided methods, e.g., the prime and/or the boost may be formulated for particular routes of administration as discussed herein.

SEQUENCES
(Farmington rhabdovirus cDNA)
SEQ ID NO: 1
ttacgacgca taagctgaga aacataagag actatgttca tagtcaccct gtattcatta 60
ttgactttta tgacctatta ttcgtgaggt catatgtgag gtaatgtcat ctgcttatgc 120
gtttgcttat aagataaaac gatagaccct tcacgggtaa atccttctcc ttgcagttct 180
cgccaagtac ctccaaagtc agacgatggc tcgtccgcta gctgctgcgc aacatctcat 240
aaccgagcgt cattcccttc aggcgactct gtcgcgggcg tccaagacca gagccgagga 300
attcgtcaaa gatttctacc ttcaagagca gtattctgtc ccgaccatcc cgacggacga 360
cattgcccag tctgggccca tgctgcttca ggccatcctg agcgaggaat acacaaaggc 420
cactgacata gcccaatcca tcctctggaa cactcccaca cccaacgggc tcctcagaga 480
gcatctagat gccgatgggg gaggctcatt cacagcgctg cccgcgtctg caatcagacc 540
cagcgacgag gcgaatgcat gggccgctcg catctccgac tcagggttgg ggcctgtctt 600
ctatgcagcc ctcgctgctt acatcatcgg ctggtcagga agaggagaga ctagccgcgt 660
gcagcagaac ataggtcaga aatggctgat gaacctgaac gcaatcttcg gcaccacgat 720
cacccatcca acaaccgtgc gtctgccaat caacgtcgtc aacaacagcc tcgcagtgag 780
gaacggactt gctgccacac tctggctata ctaccgttca tcacctcaga gtcaggacgc 840
gttcttctat gggctcatcc gtccctgttg cagtggatat ctcggcctgc tacatcgggt 900
gcaggagatt gatgagatgg agccggactt cctcagtgac ccccggatca tccaggtgaa 960
tgaggtctac agtgcactca gagccctggt tcaactggga aacgacttca agaccgccga 1020
tgatgagccc atgcaggtct gggcgtgcag gggaatcaac aacggatatc tgacatatct 1080
ctcagaaact cctgcgaaga aaggagctgt tgtgcttatg tttgcccaat gcatgctgaa 1140
gggcgactct gaggcctgga acagctaccg cactgcaacc tgggtgatgc cctattgcga 1200
caatgtggcc ctaggagcga tggcaggcta catccaagcc cgccagaaca ccagggcata 1260
tgaggtctca gcccagacag gtctcgacgt caacatggcc gcggtcaagg actttgaggc 1320
cagttcaaaa cccaaggctg ctccaatctc gctgatccca cgccccgctg atgtcgcatc 1380
ccgcacctct gagcgcccat ctattcctga ggttgacagc gacgaagagc tcggaggaat 1440
gtaaaccaat aagcttcact gccggtagtt taggcataca cacgcagttc cgttatccat 1500
cacacccgtc ccttctttta tgctgctatt atttcagttg ctaagcttcc tgatttgatt 1560
aacaaaaaac cgtagacctc ctacgtgagg tatagctaga aattggttct atcggttgag 1620
agtctttgta ctattagcca tggaggacta tttgtctagc ttagaggccg cgagagagct 1680
cgtccggacg gagctggagc ccaagcgtaa cctcatagcc agcttagagt ccgacgatcc 1740
cgatccggta atagcgccag cggtaaaacc aaaacatccc aagccatgcc tgagcactaa 1800
agaagaggat catctcccct ctcttcgcct actattcggc gcaaaacgag acacctcggt 1860
gggcgtagag cagactctcc acaagcgtct ctgcgcttgt ctcgacggtt acctgaccat 1920
gacgaagaaa gaggccaatg cctttaaggc cgcggctgaa gcagcagcat tagcagtcat 1980
ggacattaag atggagcatc agcgccagga tctagaggat ctgaccgctg ctatccctag 2040
gatagaattc aaactcaatg ccatcctgga aaacaacaag gagatagcca aggctgtaac 2100
tgctgctaag gagatggagc gggagatgtc gtggggggaa agcgccgcca gctcgctcaa 2160
gtctgtcacc ctagatgagt cgtttagggg ccctgaagag ctttcagagt catttggcat 2220
ccgatataag gtcagaacct ggaatgagtt caagaaggcg ctggaaacca gcattgtgga 2280
cctgaggcct agccctgttt catttaggga attacggact atgtggctgt ctcttgacac 2340
ctcctttagg ctcattgggt ttgccttcat tcccacatgc gagcgcctgg agaccaaagc 2400
caaatgcaag gagacaagga ctctactccc ccttgcagag tcgatcatgc gaagatggga 2460
cctgcgggat ccaaccatct tggagaaagc ctgcgtagta atgatgatcc gtgggaatga 2520
gattgcatcg ctgaatcagg taaaagatgt tctcccgacc acaattcgtg ggtggaagat 2580
cgcttattag tcactgctcc cattagtccc actagacggc atacttccat tccgcccttt 2640
aattcccctg tcagacactc atgctccgaa atcactaacc atccttgtcc accaagcaat 2700
acgcatattc agtagcactg catctcgccc tccccctatc aagccccagc gctgcagatc 2760
ttcaccacat atatacatgc atcaactaca tgtgatttag aaaaaaccag acccttcacg 2820
ggtaatagcc taactcacga acgttcctct cgtttcgtat gataaggcct taagcattgt 2880
cgatacggtc gttatgcgtc ggttcttttt aggagagagc agtgcccctg cgagggactg 2940
ggagtccgag cgacctcccc cctatgctgt tgaggtccct caaagtcacg ggataagagt 3000
caccgggtac ttccagtgca acgagcgtcc gaaatccaag aagaccctcc acagcttcgc 3060
cgtaaaactc tgcgacgcaa ttaagccggt tcgagcggat gctcccagct tgaagatagc 3120
aatatggacg gctctagatc tggccttcgt gaaacctccc aatggaactg taacaataga 3180
tgcggcggtg aaagctacac cgctaatcgg gaacacccag tacaccgtag gcgatgaaat 3240
cttccagatg ctagggagaa ggggtggcct gatcgtcatc aggaacttac cccatgatta 3300
tcctcgaacg ttgattgagt tcgcctctcc cgagccttga gcaccagggc atcggtccgc 3360
ccgccctgtg atctcccgta gccgggctca gcgatcaagc cggcccgggt cgggggggac 3420
tggtgcaaca caaggggcgg cagtggacgc tgattaacaa aaaaccacct atatagaccc 3480
ctcacggtct tagactctgt tgccagctga caaccaacac acaagacatc tctctgattc 3540
agccgacccg atcgattcct ccccacccaa ttcctaccaa cgcactcctc acaagctcca 3600
ccatgctcag gatccagatc cctccgattg ctatcattct ggtaagtctc ctcacactcg 3660
acctgtccgg tgcaaggagg acaaccacac aaagaatccc tctccttaat gattcgtggg 3720
atttgttctc gagctatggc gacattcccg aagaacttgt cgtataccag aactacagcc 3780
acaattcctc cgagttaccc cctcctggct tcgagagatg gtacataaac cgaagagtgg 3840
cagacacttc cataccgtgc aggggcccct gtctagtgcc ctacatcctt catggcctca 3900
atgacacaac tgtctctcga cggggaggag gatggcgaag gtccggaatg aagtacccaa 3960
cccacgctgt caggctaggc ccttcaacag acgacgagag agttgaggaa gacatcggct 4020
acgtcaatgt ctccgcacta tcctgcacag ggtcgcccgt tgagatggcg ataccaacaa 4080
tccccgactg caccagtgct atccatccac gatccgaggt tactgtgccc gtcaagctcg 4140
atgtcatgag acgaaatccc aactaccctc ccattagagc gtggtcgtgc atcggacaga 4200
aaatcaccaa ccgatgtgat tgggcactct tcggcgagaa cctcatatat actcaagttg 4260
aagctagctc tctagcattc aagcacacaa gagcctctct tttgaacgaa tccaacggga 4320
tagacgctga aggacgtgca gttccctata tcctcgggga tatcgaaccc gggtactgcc 4380
gaaccctatt caacacatgg gtctctagtg agatcgtgtc atgcacgccc atcgaacttg 4440
tcctagttga cctgaaccct ttgtccccgg gacatggcgg atatgctgta ttgctgccaa 4500
acggagacaa agtggatgta cacgacaagc atgcatggga tggggacaac aaaatgtgga 4560
gatgggtgta cgagaagaaa gatccctgtg cgttcgagct ggtatccagg gaagtgtgtc 4620
ttttctcact gagtaggggt agtagactga gaggagcaac ccctccccaa ggagagctcc 4680
tcacctgccc gcattcggga aaggcatttg acctgaaggg ggcccgaagg attacaccca 4740
tttcatgcaa aatcgacatg gaatatgact tgctgtcact accaaccgga gtcatcctag 4800
gcctccacct atcagaactc gggacctcct ttggcaacct ctcaatgagt cttgaaatgt 4860
atgaacctgc cacaactctg acccctgagc aaatcaactt ctcgcttaaa gagctgggaa 4920
gctggaccga ggctcaactg aagagcctgt ctcactcaat ctgcctctcc acattctcca 4980
tatgggaact atcggttggg atgatcgatc taaaccctac cagggcagca agggccttgc 5040
tccatgatga taacatactg gcaacattcg agaacggtca cttttccatc gtcagatgtc 5100
gtccggaaat agttcaagtc ccttcgcatc ctcgagcatg tcacatggat ctccgccctt 5160
atgacaagca atcacgggca tcaaccctgg tggttcccct tgacaacagc actgccctcc 5220
tggtccccga caacatcgtg gttgaaggag tagaggccag tctatgcaac cactccgttg 5280
ccatcacgct gtcgaagaac agaactcact catacagcct ctatccccag ggtcgtcctg 5340
tgcttcgaca gaaaggtgcc gtggagctcc cgacgatagg gcccctccag ttacatcctg 5400
ccactcgagt ggacctttat acactgaaag agttccagga ggaccgaata gcgcgcagtc 5460
gagtcacaga catcaaggct gccgttgacg atctgcgtgc gaagtggcgt aaaggcaaat 5520
ttgaggcgga caccacggga gggggacttt ggtcggcgat tgtgggagtc ttcagttctc 5580
tcggggggtt cttcatgagg cccttgattg ctctcgcggc gatagtgacc tcaatcatca 5640
tcctgtatat ccttctgcgt gtactgtgtg ctgcctcatg ttcgacacac cgaagagtaa 5700
ggcaggactc ttggtaaaga ggactgcgat tgttgagtgg acaaacccta ggcctattcc 5760
gatttagaaa aaaccagacc tctcacgagg tcttttctac tagctgggtt ttcctcattc 5820
tatccagagc catggccttc gacccgaact ggcagagaga aggttatgaa tgggatccgt 5880
caagtgaggg cagaccgacc gatgagaacg aagacgacag aggtcatcgg ccaaaaacga 5940
gacttcgtac attccttgcc cgcacgttaa atagccctat ccgagcccta ttctacacaa 6000
tattcctagg aattcgagcg gtttgggacg ggttcaaaag actcctacct gtgaggaccg 6060
aaaagggtta tgcgaggttt tctgagtgcg tcacatatgg aatgatcgga tgtgatgagt 6120
gtgtaataga cccggtgagg gttgtcattg agctgaccga gatgcagtta ccgattaaag 6180
gcaaaggctc tacgaggttg agagcaatga taactgaaga ccttctcacg gggatgcgca 6240
cagccgtgcc tcagatcaga gtgagatcga agatcctagc agagcggtta gggagagcaa 6300
tcggccgaga gaccttgccg gcaatgatcc atcatgagtg ggcatttgtg atggggaaga 6360
ttctcacttt catggcagac aatgtgggta tgaacgctga cacggtcgag ggcgttctat 6420
cactatcaga ggtcacacgg cgatgggata tcggcaactc tgtgtccgca gtgttcaatc 6480
ctgatggcct tactatcaga gtagaaaaca cgggttacat catgaccaga gagactgcct 6540
gcatgatcgg agacattcat gctcaatttg caatccaata cctagctgca tacctagacg 6600
aggtgatcgg cacaaggacg tctctctcac ccgccgaact gacctctctc aaactatggg 6660
gacttaacgt cctgaaactc ctaggacgga acggttatga ggtgatcgcc tgcatggagc 6720
ccatagggta cgctgtcctg atgatgggaa gagacaggag tcctgatccc tatgtcaatg 6780
acacctattt aaacagcatc ctctcagaat tccctgtcga ctctgacgct cgagcctgcg 6840
ttgaagccct cttaactatc tatatgagct tcggcacacc ccataaagtc tcggacgcat 6900
tcggcctctt cagaatgttg ggacatccga tggttgatgg agctgacggg attgaaaaga 6960
tgcgaaggtt aagcaagaag gtcaagatcc cagaccagtc tacagcgatc gacctcgggg 7020
ctatcatggc cgaactgttt gtgcggagtt tcgtaaagaa gcacaaaagg tggcccaact 7080
gctccatcaa tctcccgcca cgacacccct tccaccacgc ccgcctatgt gggtatgtcc 7140
cggctgaaac ccatccccta aacaacactg catcctgggc ggctgtggag ttcaaccagg 7200
aattcgagcc gccgagacag tacaaccttg cagacatcat tgatgacaag tcgtgctctc 7260
ccaacaagca tgagctatat ggtgcttgga tgaagtcaaa aacagctggg tggcaggaac 7320
aaaagaagct catactccga tggttcactg agaccatggt taaaccttcg gagctcctgg 7380
aagagattga tgcacacggc ttccgagaag aggataagtt gattggatta acaccaaagg 7440
agagagagct gaaattaaca ccaagaatgt tctccttgat gacattcaag ttcagaacct 7500
accaagtcct cactgagagt atggtcgccg atgagatcct cccgcacttc ccccagatca 7560
ccatgaccat gtccaaccac gaactcacaa agaggttgat tagcagaacg agacctcaat 7620
ctggaggagg gcgtgatgtt cacatcaccg tgaacataga tttccagaaa tggaacacaa 7680
acatgagaca cggactggtc aaacatgtct tcgagcgact ggacaacctc tttggcttca 7740
ccaacttaat cagacgaact catgaatact tccaggaggc gaaatactat ctggctgaag 7800
atggaactaa tctgtcgttc gacaggaacg gggagttaat agatggccca tacgtttaca 7860
ccggatcata cggggggaac gaggggttac gacagaagcc ctggacaata gttaccgtgt 7920
gtggaatata caaggtagct agagacctga aaatcaaaca tcagatcacc ggtcagggag 7980
ataatcaggt ggtcacccta atatttccgg atcgagagtt gccttcagat ccggtggaga 8040
ggagcaagta ctgtagagac aagagcagtc agttcctgac acgtctcagt caatatttcg 8100
ctgaggttgg tttgcccgtc aagactgaag agacatggat gtcatcacgt ctctatgctt 8160
acggtaagcg catgttctta gagggagttc cacttaagat gtttctcaag aagataggca 8220
gagctttcgc cctctcgaat gagtttgtcc cgtccctcga ggaagatctg gccagagtct 8280
ggagtgccac cagcgcagcg gtagagcttg acctaactcc ctacgtagga tatgtcctcg 8340
ggtgctgctt gtctgcgcag gcgatcagaa atcacctcat ctactcccct gttctggagg 8400
gccctctgct ggttaaggcc tacgagcgta agttcattaa ctacgacgga ggaacaaagc 8460
ggggggcgat gcccggccta cgtccaacct ttgagagcct agtcaaaagt atctgctgga 8520
agccaaaggc catcggaggg tggccggtat tgatgttaga agatctcatc atcaaagggt 8580
tccctgatcc ggcgactagc gccctggctc aattgaagtc aatggtgcca tatacctctg 8640
gtatcgaccg ggagatcata ctttcctgtc tcaaccttcc cttatcgtcg gtggtatctc 8700
cgtcaatgtt gttaaaggac ccggcggcca tcaacaccat cacaaccccg tccgcgggcg 8760
acatcctgca agaggtcgcc agagactatg ttaccgatta cccactccaa aacccgcagc 8820
tcagagcagt ggtcaagaac gtgaagaccg agctagacac attggccagt gacttattca 8880
aatgtgaacc tttctttcct cctttaatga gcgatatctt ctcggcatct ctcccggcat 8940
atcaagacag gattgttcgc aagtgctcca cgacttctac aatcaggaga aaagctgccg 9000
agaggggctc cgactctctc ctcaaccgga tgaaaaggaa tgagatcaat aagatgatgt 9060
tacatctttg ggctacctgg ggaaggagcc ctctggccag attagacacc agatgtctca 9120
caacctgcac caagcaatta gcccaacagt atcggaacca gtcttgggga aagcagatcc 9180
atggagtctc agtcggccac cccttagaac tgttcggtcg aataacaccc agccatagat 9240
gcctacatga ggaggaccac ggagatttcc tgcaaacctt cgccagcgag catgtgaacc 9300
aagtggacac cgacatcacc acaactctgg ggccgttcta cccttacata ggctcggaga 9360
cgcgagaacg ggcagtcaag gttcgaaaag gagtgaatta cgtagttgag ccgcttctga 9420
aacccgcagt tcgactacta agagccatta attggttcat tcccgaggag tcagatgcgt 9480
cccatttgct gagcaatcta ttagcgtctg ttaccgacat caatcctcaa gaccactact 9540
catctaccga agtagggggg ggcaacgccg tccatcgcta cagctgccga ctatccgaca 9600
aattgagcag agtcaacaac ttatatcagt tgcatactta tttatctgtc acaacagagc 9660
ggttgaccaa gtacagtcga ggatcaaaaa acactgacgc acacttccag agcatgatga 9720
tttatgcaca aagccgtcat atagacctca tcttggagtc tctgcacacc ggagagatgg 9780
taccgttgga gtgtcatcat cacattgagt gcaatcactg tatagaggat atacccgacg 9840
agccaatcac gggggacccg gcttggactg aagtcaagtt tccttcaagt cctcaggagc 9900
cctttcttta catcaggcaa caagatctgc cggtcaaaga caaactcgag cctgtgcctc 9960
gcatgaacat cgtccgtctt gccggattgg gtccggaggc gattagtgag ctagcgcact 10020
actttgttgc attccgagtt atccgggcgt cagagacgga tgtcgaccct aacgatgttc 10080
tctcgtggac ctggctgagc cgaattgatc ctgacaaatt ggttgagtat atcgtgcatg 10140
tgttcgcttc actggaatgg catcatgtat taatgtcagg cgtgagtgtg agcgtcagag 10200
atgcattctt taagatgcta gtgtctaaaa gaatctcaga gactccgcta agttcattct 10260
attatctggc caacctgttc gttgaccctc agactcgcga agcactaatg agctctaaat 10320
acgggttcag cccccccgcc gagacagtcc ccaacgcaaa tgccgccgca gccgaaataa 10380
gaagatgctg tgcgaacagt gcgccgtcga tcttagaatc agcccttcac agccgtgagg 10440
ttgtttggat gccaggaacg aacaattatg gagacgttgt catctggtct cattacatta 10500
gattacggtt cagcgaagtt aaactagttg acattacacg atatcagcag tggtggagac 10560
agtctgagcg agacccctac gatttggtcc cggacatgca ggttcttgag agcgacctag 10620
atacgctgat gaaacggata ccgaggctca tgcgcaaggc gagacgtccc cctcttcagg 10680
taattcgaga ggacctggat gtcgcagtca tcaatgctga tcatcccgct cactctgtgc 10740
ttcagaacaa atacaggaaa ttgattttca gagagccgaa gattatcacg ggagctgtgt 10800
acaagtacct ctccctaaaa tcagagttga cagagttcac ctcagcaatg gtgatcggag 10860
acggaactgg aggtatcacc gccgccatga tggccgatgg gatagatgtg tggtatcaga 10920
cgctcgtcaa ctatgaccac gtgacacaac agggattatc cgtacaagcc ccggcagcat 10980
tggatcttct gcgcggggca ccctctggta ggctcttgaa tccgggaaga ttcgcatcat 11040
ttgggtctga cctaactgac cctcgattta cagcctactt tgatcaatat cccccgttca 11100
aggtggacac tctatggtct gacgcagagg gcgacttttg ggacaagcct tccaagttga 11160
atcaatactt tgagaacatc attgctttga gacatcggtt cgtgaagaca aatggacagc 11220
ttgtcgtgaa ggtgtatctg actcaagaca ctgctaccac aattgaagca ttcagaaaga 11280
agctgtcccc atgcgccatc atcgtgtctc tcttctcgac ggaaggctcc acagaatgct 11340
tcgtcctaag caatctcatc gcaccagaca cccctgtcga ccttgagatg gtggagaata 11400
tccctaaact aacatccctt gttccccaga ggacgacagt gaaatgctat tcccgacgag 11460
tagcgtgcat cagtaaaagg tggggacttt tcagatctcc gagcatagcc cttgaagtcc 11520
aaccgttcct tcactacatc acaaaggtca tctcagacaa aggaacacaa ctgagtctca 11580
tggcggtagc tgacacaatg atcaacagtt acaagaaggc tatctcaccc cgagtgttcg 11640
atctacaccg gcatagggcc gcactgggtt tcgggaggag atccttgcat ctcatctggg 11700
ggatgatcat ctcaccaatc gcttaccagc attttgagaa tccggccaag ttgatggatg 11760
tcctggacat gttgaccaat aacatctcag ctttcttatc gatatcgtcg tcaggatttg 11820
acctgtcatt tagtgtcagt gcagaccgag atgtccggat tgacagcaaa cttgtcagac 11880
tcccgctatt cgaaggatca gacctaaaat tcatgaaaac catcatgtct accctcggat 11940
ctgtgttcaa ccaggtcgag ccttttaagg ggatcgccat aaacccttct aaactaatga 12000
ctgtcaagag gacacaggag ttacgttaca acaacctaat ttacactaag gatgccatcc 12060
tattccccaa tgaagcggca aaaaacactg ccccgcttcg agccaacatg gtataccccg 12120
tccggggaga tctattcgcc cctaccgatc gcataccaat catgactcta gtcagcgatg 12180
agacaacacc tcagcactct cctccagagg atgaggcata actgaatcct ccctgaaggc 12240
tcacatgtcc cacgcgacgc aagatataac gacaagcaac tcgccctatt aactgtgatt 12300
aataaaaaac cgattattca gttgcttgag ggagtttcaa tccgttcagt gtatgatagg 12360
aagtttctga gatggtgggg attagggggc acctagagta tgtttgttcg ttttatgcgt 12420
cgt 12423
(Farmington rhabdovirus RNA)
SEQ ID NO: 2
uuacgacgca uaagcugaga aacauaagag acuauguuca uagucacccu guauucauua 60
uugacuuuua ugaccuauua uucgugaggu cauaugugag guaaugucau cugcuuaugc 120
guuugcuuau aagauaaaac gauagacccu ucacggguaa auccuucucc uugcaguucu 180
cgccaaguac cuccaaaguc agacgauggc ucguccgcua gcugcugcgc aacaucucau 240
aaccgagcgu cauucccuuc aggcgacucu gucgcgggcg uccaagacca gagccgagga 300
auucgucaaa gauuucuacc uucaagagca guauucuguc ccgaccaucc cgacggacga 360
cauugcccag ucugggccca ugcugcuuca ggccauccug agcgaggaau acacaaaggc 420
cacugacaua gcccaaucca uccucuggaa cacucccaca cccaacgggc uccucagaga 480
gcaucuagau gccgaugggg gaggcucauu cacagcgcug cccgcgucug caaucagacc 540
cagcgacgag gcgaaugcau gggccgcucg caucuccgac ucaggguugg ggccugucuu 600
cuaugcagcc cucgcugcuu acaucaucgg cuggucagga agaggagaga cuagccgcgu 660
gcagcagaac auaggucaga aauggcugau gaaccugaac gcaaucuucg gcaccacgau 720
cacccaucca acaaccgugc gucugccaau caacgucguc aacaacagcc ucgcagugag 780
gaacggacuu gcugccacac ucuggcuaua cuaccguuca ucaccucaga gucaggacgc 840
guucuucuau gggcucaucc gucccuguug caguggauau cucggccugc uacaucgggu 900
gcaggagauu gaugagaugg agccggacuu ccucagugac ccccggauca uccaggugaa 960
ugaggucuac agugcacuca gagcccuggu ucaacuggga aacgacuuca agaccgccga 1020
ugaugagccc augcaggucu gggcgugcag gggaaucaac aacggauauc ugacauaucu 1080
cucagaaacu ccugcgaaga aaggagcugu ugugcuuaug uuugcccaau gcaugcugaa 1140
gggcgacucu gaggccugga acagcuaccg cacugcaacc ugggugaugc ccuauugcga 1200
caauguggcc cuaggagcga uggcaggcua cauccaagcc cgccagaaca ccagggcaua 1260
ugaggucuca gcccagacag gucucgacgu caacauggcc gcggucaagg acuuugaggc 1320
caguucaaaa cccaaggcug cuccaaucuc gcugauccca cgccccgcug augucgcauc 1380
ccgcaccucu gagcgcccau cuauuccuga gguugacagc gacgaagagc ucggaggaau 1440
guaaaccaau aagcuucacu gccgguaguu uaggcauaca cacgcaguuc cguuauccau 1500
cacacccguc ccuucuuuua ugcugcuauu auuucaguug cuaagcuucc ugauuugauu 1560
aacaaaaaac cguagaccuc cuacgugagg uauagcuaga aauugguucu aucgguugag 1620
agucuuugua cuauuagcca uggaggacua uuugucuagc uuagaggccg cgagagagcu 1680
cguccggacg gagcuggagc ccaagcguaa ccucauagcc agcuuagagu ccgacgaucc 1740
cgauccggua auagcgccag cgguaaaacc aaaacauccc aagccaugcc ugagcacuaa 1800
agaagaggau caucuccccu cucuucgccu acuauucggc gcaaaacgag acaccucggu 1860
gggcguagag cagacucucc acaagcgucu cugcgcuugu cucgacgguu accugaccau 1920
gacgaagaaa gaggccaaug ccuuuaaggc cgcggcugaa gcagcagcau uagcagucau 1980
ggacauuaag auggagcauc agcgccagga ucuagaggau cugaccgcug cuaucccuag 2040
gauagaauuc aaacucaaug ccauccugga aaacaacaag gagauagcca aggcuguaac 2100
ugcugcuaag gagauggagc gggagauguc guggggggaa agcgccgcca gcucgcucaa 2160
gucugucacc cuagaugagu cguuuagggg cccugaagag cuuucagagu cauuuggcau 2220
ccgauauaag gucagaaccu ggaaugaguu caagaaggcg cuggaaacca gcauugugga 2280
ccugaggccu agcccuguuu cauuuaggga auuacggacu auguggcugu cucuugacac 2340
cuccuuuagg cucauugggu uugccuucau ucccacaugc gagcgccugg agaccaaagc 2400
caaaugcaag gagacaagga cucuacuccc ccuugcagag ucgaucaugc gaagauggga 2460
ccugcgggau ccaaccaucu uggagaaagc cugcguagua augaugaucc gugggaauga 2520
gauugcaucg cugaaucagg uaaaagaugu ucucccgacc acaauucgug gguggaagau 2580
cgcuuauuag ucacugcucc cauuaguccc acuagacggc auacuuccau uccgcccuuu 2640
aauuccccug ucagacacuc augcuccgaa aucacuaacc auccuugucc accaagcaau 2700
acgcauauuc aguagcacug caucucgccc ucccccuauc aagccccagc gcugcagauc 2760
uucaccacau auauacaugc aucaacuaca ugugauuuag aaaaaaccag acccuucacg 2820
gguaauagcc uaacucacga acguuccucu cguuucguau gauaaggccu uaagcauugu 2880
cgauacgguc guuaugcguc gguucuuuuu aggagagagc agugccccug cgagggacug 2940
ggaguccgag cgaccucccc ccuaugcugu ugaggucccu caaagucacg ggauaagagu 3000
caccggguac uuccagugca acgagcgucc gaaauccaag aagacccucc acagcuucgc 3060
cguaaaacuc ugcgacgcaa uuaagccggu ucgagcggau gcucccagcu ugaagauagc 3120
aauauggacg gcucuagauc uggccuucgu gaaaccuccc aauggaacug uaacaauaga 3180
ugcggcggug aaagcuacac cgcuaaucgg gaacacccag uacaccguag gcgaugaaau 3240
cuuccagaug cuagggagaa gggguggccu gaucgucauc aggaacuuac cccaugauua 3300
uccucgaacg uugauugagu ucgccucucc cgagccuuga gcaccagggc aucgguccgc 3360
ccgcccugug aucucccgua gccgggcuca gcgaucaagc cggcccgggu cgggggggac 3420
uggugcaaca caaggggcgg caguggacgc ugauuaacaa aaaaccaccu auauagaccc 3480
cucacggucu uagacucugu ugccagcuga caaccaacac acaagacauc ucucugauuc 3540
agccgacccg aucgauuccu ccccacccaa uuccuaccaa cgcacuccuc acaagcucca 3600
ccaugcucag gauccagauc ccuccgauug cuaucauucu gguaagucuc cucacacucg 3660
accuguccgg ugcaaggagg acaaccacac aaagaauccc ucuccuuaau gauucguggg 3720
auuuguucuc gagcuauggc gacauucccg aagaacuugu cguauaccag aacuacagcc 3780
acaauuccuc cgaguuaccc ccuccuggcu ucgagagaug guacauaaac cgaagagugg 3840
cagacacuuc cauaccgugc aggggccccu gucuagugcc cuacauccuu cauggccuca 3900
augacacaac ugucucucga cggggaggag gauggcgaag guccggaaug aaguacccaa 3960
cccacgcugu caggcuaggc ccuucaacag acgacgagag aguugaggaa gacaucggcu 4020
acgucaaugu cuccgcacua uccugcacag ggucgcccgu ugagauggcg auaccaacaa 4080
uccccgacug caccagugcu auccauccac gauccgaggu uacugugccc gucaagcucg 4140
augucaugag acgaaauccc aacuacccuc ccauuagagc guggucgugc aucggacaga 4200
aaaucaccaa ccgaugugau ugggcacucu ucggcgagaa ccucauauau acucaaguug 4260
aagcuagcuc ucuagcauuc aagcacacaa gagccucucu uuugaacgaa uccaacggga 4320
uagacgcuga aggacgugca guucccuaua uccucgggga uaucgaaccc ggguacugcc 4380
gaacccuauu caacacaugg gucucuagug agaucguguc augcacgccc aucgaacuug 4440
uccuaguuga ccugaacccu uuguccccgg gacauggcgg auaugcugua uugcugccaa 4500
acggagacaa aguggaugua cacgacaagc augcauggga uggggacaac aaaaugugga 4560
gaugggugua cgagaagaaa gaucccugug cguucgagcu gguauccagg gaaguguguc 4620
uuuucucacu gaguaggggu aguagacuga gaggagcaac cccuccccaa ggagagcucc 4680
ucaccugccc gcauucggga aaggcauuug accugaaggg ggcccgaagg auuacaccca 4740
uuucaugcaa aaucgacaug gaauaugacu ugcugucacu accaaccgga gucauccuag 4800
gccuccaccu aucagaacuc gggaccuccu uuggcaaccu cucaaugagu cuugaaaugu 4860
augaaccugc cacaacucug accccugagc aaaucaacuu cucgcuuaaa gagcugggaa 4920
gcuggaccga ggcucaacug aagagccugu cucacucaau cugccucucc acauucucca 4980
uaugggaacu aucgguuggg augaucgauc uaaacccuac cagggcagca agggccuugc 5040
uccaugauga uaacauacug gcaacauucg agaacgguca cuuuuccauc gucagauguc 5100
guccggaaau aguucaaguc ccuucgcauc cucgagcaug ucacauggau cuccgcccuu 5160
augacaagca aucacgggca ucaacccugg ugguuccccu ugacaacagc acugcccucc 5220
ugguccccga caacaucgug guugaaggag uagaggccag ucuaugcaac cacuccguug 5280
ccaucacgcu gucgaagaac agaacucacu cauacagccu cuauccccag ggucguccug 5340
ugcuucgaca gaaaggugcc guggagcucc cgacgauagg gccccuccag uuacauccug 5400
ccacucgagu ggaccuuuau acacugaaag aguuccagga ggaccgaaua gcgcgcaguc 5460
gagucacaga caucaaggcu gccguugacg aucugcgugc gaaguggcgu aaaggcaaau 5520
uugaggcgga caccacggga gggggacuuu ggucggcgau ugugggaguc uucaguucuc 5580
ucgggggguu cuucaugagg cccuugauug cucucgcggc gauagugacc ucaaucauca 5640
uccuguauau ccuucugcgu guacugugug cugccucaug uucgacacac cgaagaguaa 5700
ggcaggacuc uugguaaaga ggacugcgau uguugagugg acaaacccua ggccuauucc 5760
gauuuagaaa aaaccagacc ucucacgagg ucuuuucuac uagcuggguu uuccucauuc 5820
uauccagagc cauggccuuc gacccgaacu ggcagagaga agguuaugaa ugggauccgu 5880
caagugaggg cagaccgacc gaugagaacg aagacgacag aggucaucgg ccaaaaacga 5940
gacuucguac auuccuugcc cgcacguuaa auagcccuau ccgagcccua uucuacacaa 6000
uauuccuagg aauucgagcg guuugggacg gguucaaaag acuccuaccu gugaggaccg 6060
aaaaggguua ugcgagguuu ucugagugcg ucacauaugg aaugaucgga ugugaugagu 6120
guguaauaga cccggugagg guugucauug agcugaccga gaugcaguua ccgauuaaag 6180
gcaaaggcuc uacgagguug agagcaauga uaacugaaga ccuucucacg gggaugcgca 6240
cagccgugcc ucagaucaga gugagaucga agauccuagc agagcgguua gggagagcaa 6300
ucggccgaga gaccuugccg gcaaugaucc aucaugagug ggcauuugug auggggaaga 6360
uucucacuuu cauggcagac aaugugggua ugaacgcuga cacggucgag ggcguucuau 6420
cacuaucaga ggucacacgg cgaugggaua ucggcaacuc uguguccgca guguucaauc 6480
cugauggccu uacuaucaga guagaaaaca cggguuacau caugaccaga gagacugccu 6540
gcaugaucgg agacauucau gcucaauuug caauccaaua ccuagcugca uaccuagacg 6600
aggugaucgg cacaaggacg ucucucucac ccgccgaacu gaccucucuc aaacuauggg 6660
gacuuaacgu ccugaaacuc cuaggacgga acgguuauga ggugaucgcc ugcauggagc 6720
ccauagggua cgcuguccug augaugggaa gagacaggag uccugauccc uaugucaaug 6780
acaccuauuu aaacagcauc cucucagaau ucccugucga cucugacgcu cgagccugcg 6840
uugaagcccu cuuaacuauc uauaugagcu ucggcacacc ccauaaaguc ucggacgcau 6900
ucggccucuu cagaauguug ggacauccga ugguugaugg agcugacggg auugaaaaga 6960
ugcgaagguu aagcaagaag gucaagaucc cagaccaguc uacagcgauc gaccucgggg 7020
cuaucauggc cgaacuguuu gugcggaguu ucguaaagaa gcacaaaagg uggcccaacu 7080
gcuccaucaa ucucccgcca cgacaccccu uccaccacgc ccgccuaugu ggguaugucc 7140
cggcugaaac ccauccccua aacaacacug cauccugggc ggcuguggag uucaaccagg 7200
aauucgagcc gccgagacag uacaaccuug cagacaucau ugaugacaag ucgugcucuc 7260
ccaacaagca ugagcuauau ggugcuugga ugaagucaaa aacagcuggg uggcaggaac 7320
aaaagaagcu cauacuccga ugguucacug agaccauggu uaaaccuucg gagcuccugg 7380
aagagauuga ugcacacggc uuccgagaag aggauaaguu gauuggauua acaccaaagg 7440
agagagagcu gaaauuaaca ccaagaaugu ucuccuugau gacauucaag uucagaaccu 7500
accaaguccu cacugagagu auggucgccg augagauccu cccgcacuuc ccccagauca 7560
ccaugaccau guccaaccac gaacucacaa agagguugau uagcagaacg agaccucaau 7620
cuggaggagg gcgugauguu cacaucaccg ugaacauaga uuuccagaaa uggaacacaa 7680
acaugagaca cggacugguc aaacaugucu ucgagcgacu ggacaaccuc uuuggcuuca 7740
ccaacuuaau cagacgaacu caugaauacu uccaggaggc gaaauacuau cuggcugaag 7800
auggaacuaa ucugucguuc gacaggaacg gggaguuaau agauggccca uacguuuaca 7860
ccggaucaua cggggggaac gagggguuac gacagaagcc cuggacaaua guuaccgugu 7920
guggaauaua caagguagcu agagaccuga aaaucaaaca ucagaucacc ggucagggag 7980
auaaucaggu ggucacccua auauuuccgg aucgagaguu gccuucagau ccgguggaga 8040
ggagcaagua cuguagagac aagagcaguc aguuccugac acgucucagu caauauuucg 8100
cugagguugg uuugcccguc aagacugaag agacauggau gucaucacgu cucuaugcuu 8160
acgguaagcg cauguucuua gagggaguuc cacuuaagau guuucucaag aagauaggca 8220
gagcuuucgc ccucucgaau gaguuugucc cgucccucga ggaagaucug gccagagucu 8280
ggagugccac cagcgcagcg guagagcuug accuaacucc cuacguagga uauguccucg 8340
ggugcugcuu gucugcgcag gcgaucagaa aucaccucau cuacuccccu guucuggagg 8400
gcccucugcu gguuaaggcc uacgagcgua aguucauuaa cuacgacgga ggaacaaagc 8460
ggggggcgau gcccggccua cguccaaccu uugagagccu agucaaaagu aucugcugga 8520
agccaaaggc caucggaggg uggccgguau ugauguuaga agaucucauc aucaaagggu 8580
ucccugaucc ggcgacuagc gcccuggcuc aauugaaguc aauggugcca uauaccucug 8640
guaucgaccg ggagaucaua cuuuccuguc ucaaccuucc cuuaucgucg gugguaucuc 8700
cgucaauguu guuaaaggac ccggcggcca ucaacaccau cacaaccccg uccgcgggcg 8760
acauccugca agaggucgcc agagacuaug uuaccgauua cccacuccaa aacccgcagc 8820
ucagagcagu ggucaagaac gugaagaccg agcuagacac auuggccagu gacuuauuca 8880
aaugugaacc uuucuuuccu ccuuuaauga gcgauaucuu cucggcaucu cucccggcau 8940
aucaagacag gauuguucgc aagugcucca cgacuucuac aaucaggaga aaagcugccg 9000
agaggggcuc cgacucucuc cucaaccgga ugaaaaggaa ugagaucaau aagaugaugu 9060
uacaucuuug ggcuaccugg ggaaggagcc cucuggccag auuagacacc agaugucuca 9120
caaccugcac caagcaauua gcccaacagu aucggaacca gucuugggga aagcagaucc 9180
auggagucuc agucggccac cccuuagaac uguucggucg aauaacaccc agccauagau 9240
gccuacauga ggaggaccac ggagauuucc ugcaaaccuu cgccagcgag caugugaacc 9300
aaguggacac cgacaucacc acaacucugg ggccguucua cccuuacaua ggcucggaga 9360
cgcgagaacg ggcagucaag guucgaaaag gagugaauua cguaguugag ccgcuucuga 9420
aacccgcagu ucgacuacua agagccauua auugguucau ucccgaggag ucagaugcgu 9480
cccauuugcu gagcaaucua uuagcgucug uuaccgacau caauccucaa gaccacuacu 9540
caucuaccga aguagggggg ggcaacgccg uccaucgcua cagcugccga cuauccgaca 9600
aauugagcag agucaacaac uuauaucagu ugcauacuua uuuaucuguc acaacagagc 9660
gguugaccaa guacagucga ggaucaaaaa acacugacgc acacuuccag agcaugauga 9720
uuuaugcaca aagccgucau auagaccuca ucuuggaguc ucugcacacc ggagagaugg 9780
uaccguugga gugucaucau cacauugagu gcaaucacug uauagaggau auacccgacg 9840
agccaaucac gggggacccg gcuuggacug aagucaaguu uccuucaagu ccucaggagc 9900
ccuuucuuua caucaggcaa caagaucugc cggucaaaga caaacucgag ccugugccuc 9960
gcaugaacau cguccgucuu gccggauugg guccggaggc gauuagugag cuagcgcacu 10020
acuuuguugc auuccgaguu auccgggcgu cagagacgga ugucgacccu aacgauguuc 10080
ucucguggac cuggcugagc cgaauugauc cugacaaauu gguugaguau aucgugcaug 10140
uguucgcuuc acuggaaugg caucauguau uaaugucagg cgugagugug agcgucagag 10200
augcauucuu uaagaugcua gugucuaaaa gaaucucaga gacuccgcua aguucauucu 10260
auuaucuggc caaccuguuc guugacccuc agacucgcga agcacuaaug agcucuaaau 10320
acggguucag cccccccgcc gagacagucc ccaacgcaaa ugccgccgca gccgaaauaa 10380
gaagaugcug ugcgaacagu gcgccgucga ucuuagaauc agcccuucac agccgugagg 10440
uuguuuggau gccaggaacg aacaauuaug gagacguugu caucuggucu cauuacauua 10500
gauuacgguu cagcgaaguu aaacuaguug acauuacacg auaucagcag ugguggagac 10560
agucugagcg agaccccuac gauuuggucc cggacaugca gguucuugag agcgaccuag 10620
auacgcugau gaaacggaua ccgaggcuca ugcgcaaggc gagacguccc ccucuucagg 10680
uaauucgaga ggaccuggau gucgcaguca ucaaugcuga ucaucccgcu cacucugugc 10740
uucagaacaa auacaggaaa uugauuuuca gagagccgaa gauuaucacg ggagcugugu 10800
acaaguaccu cucccuaaaa ucagaguuga cagaguucac cucagcaaug gugaucggag 10860
acggaacugg agguaucacc gccgccauga uggccgaugg gauagaugug ugguaucaga 10920
cgcucgucaa cuaugaccac gugacacaac agggauuauc cguacaagcc ccggcagcau 10980
uggaucuucu gcgcggggca cccucuggua ggcucuugaa uccgggaaga uucgcaucau 11040
uugggucuga ccuaacugac ccucgauuua cagccuacuu ugaucaauau cccccguuca 11100
agguggacac ucuauggucu gacgcagagg gcgacuuuug ggacaagccu uccaaguuga 11160
aucaauacuu ugagaacauc auugcuuuga gacaucgguu cgugaagaca aauggacagc 11220
uugucgugaa gguguaucug acucaagaca cugcuaccac aauugaagca uucagaaaga 11280
agcugucccc augcgccauc aucgugucuc ucuucucgac ggaaggcucc acagaaugcu 11340
ucguccuaag caaucucauc gcaccagaca ccccugucga ccuugagaug guggagaaua 11400
ucccuaaacu aacaucccuu guuccccaga ggacgacagu gaaaugcuau ucccgacgag 11460
uagcgugcau caguaaaagg uggggacuuu ucagaucucc gagcauagcc cuugaagucc 11520
aaccguuccu ucacuacauc acaaagguca ucucagacaa aggaacacaa cugagucuca 11580
uggcgguagc ugacacaaug aucaacaguu acaagaaggc uaucucaccc cgaguguucg 11640
aucuacaccg gcauagggcc gcacuggguu ucgggaggag auccuugcau cucaucuggg 11700
ggaugaucau cucaccaauc gcuuaccagc auuuugagaa uccggccaag uugauggaug 11760
uccuggacau guugaccaau aacaucucag cuuucuuauc gauaucgucg ucaggauuug 11820
accugucauu uagugucagu gcagaccgag auguccggau ugacagcaaa cuugucagac 11880
ucccgcuauu cgaaggauca gaccuaaaau ucaugaaaac caucaugucu acccucggau 11940
cuguguucaa ccaggucgag ccuuuuaagg ggaucgccau aaacccuucu aaacuaauga 12000
cugucaagag gacacaggag uuacguuaca acaaccuaau uuacacuaag gaugccaucc 12060
uauuccccaa ugaagcggca aaaaacacug ccccgcuuc gagccaacaug guauaccccg 12120
uccggggaga ucuauucgcc ccuaccgauc gcauaccaau caugacucua gucagcgaug 12180
agacaacacc ucagcacucu ccuccagagg augaggcaua acugaauccu cccugaaggc 12240
ucacaugucc cacgcgacgc aagauauaac gacaagcaac ucgcccuauu aacugugauu 12300
aauaaaaaac cgauuauuca guugcuugag ggaguuucaa uccguucagu guaugauagg 12360
aaguuucuga gauggugggg auuagggggc accuagagua uguuuguucg uuuuaugcgu 12420
cgu 12423
(Farmington rhabdovirus ORF1 protein)
SEQ ID NO: 3
MARPLAAAQHLITERHSLQATLSRASKTRAEEFVKDFYLQEQYSVPTIPTDDIAQSGPML
LQAILSEEYTKATDIAQSILWNTPTPNGLLREHLDADGGGSFTALPASAIRPSDEANAWA
ARISDSGLGPVFYAALAAYIIGWSGRGETSRVQQNIGQKWLMNLNAIFGTTITHPTTVRL
PINVVNNSLAVRNGLAATLWLYYRSSPQSQDAFFYGLIRPCCSGYLGLLHRVQEIDEMEP
DFLSDPRIIQVNEVYSALRALVQLGNDFKTADDEPMQVWACRGINNGYLTYLSETPAKKG
AVVLMFAQCMLKGDSEAWNSYRTATWVMPYCDNVALGAMAGYIQARQNTRAYEVSAQTGL
DVNMAAVKDFEASSKPKAAPISLIPRPADVASRTSERPSIPEVDSDEELGGM
(Farmington rhabdovirus ORF2 protein)
SEQ ID NO: 4
MEDYLSSLEAARELVRTELEPKRNLIASLESDDPDPVIAPAVKPKHPKPCLSTKEEDHLP
SLRLLFGAKRDTSVGVEQTLHKRLCACLDGYLTMTKKEANAFKAAAEAAALAVMDIKMEH
QRQDLEDLTAAIPRIEFKLNAILENNKEIAKAVTAAKEMEREMSWGESAASSLKSVTLDE
SFRGPEELSESFGIRYKVRTWNEFKKALETSIVDLRPSPVSFRELRTMWLSLDTSFRLIG
FAFIPTCERLETKAKCKETRTLLPLAESIMRRWDLRDPTILEKACVVMMIRGNEIASLNQ
VKDVLPTTIRGWKIAY
(Farmington rhabdovirus ORF3 protein)
SEQ ID NO: 5
MRRFFLGESSAPARDWESERPPPYAVEVPQSHGIRVTGYFQCNERPKSKKTLHSFAVKLC
DAIKPVRADAPSLKIAIWTALDLAFVKPPNGTVTIDAAVKATPLIGNTQYTVGDEIFQML
GRRGGLIVIRNLPHDYPRTLIEFASPEP
(Farmington rhabdovirus ORF4 protein)
SEQ ID NO: 6
MLRIQIPPIAIILVSLLTLDLSGARRTTTQRIPLLNDSWDLFSSYGDIPEELVVYQNYSH
NSSELPPPGFERWYINRRVADTSIPCRGPCLVPYILHGLNDTTVSRRGGGWRRSGMKYPT
HAVRLGPSTDDERVEEDIGYVNVSALSCTGSPVEMAIPTIPDCTSAIHPRSEVTVPVKLD
VMRRNPNYPPIRAWSCIGQKITNRCDWALFGENLIYTQVEASSLAFKHTRASLLNESNGI
DAEGRAVPYILGDIEPGYCRTLFNTWVSSEIVSCTPIELVLVDLNPLSPGHGGYAVLLPN
GDKVDVHDKHAWDGDNKMWRWVYEKKDPCAFELVSREVCLFSLSRGSRLRGATPPQGELL
TCPHSGKAFDLKGARRITPISCKIDMEYDLLSLPTGVILGLHLSELGTSFGNLSMSLEMY
EPATTLTPEQINFSLKELGSWTEAQLKSLSHSICLSTFSIWELSVGMIDLNPTRAARALL
HDDNILATFENGHFSIVRCRPEIVQVPSHPRACHMDLRPYDKQSRASTLVVPLDNSTALL
VPDNIVVEGVEASLCNHSVAITLSKNRTHSYSLYPQGRPVLRQKGAVELPTIGPLQLHPA
TRVDLYTLKEFQEDRIARSRVTDIKAAVDDLRAKWRKGKFEADTTGGGLWSAIVGVFSSL
GGFFMRPLIALAAIVTSIIILYILLRVLCAASCSTHRRVRQDSW
(Farmington rhabdovirus ORF5 protein)
SEQ ID NO: 7
MAFDPNWQREGYEWDPSSEGRPTDENEDDRGHRPKTRLRTFLARTLNSPIRALFYTIFLG
IRAVWDGFKRLLPVRTEKGYARFSECVTYGMIGCDECVIDPVRVVIELTEMQLPIKGKGS
TRLRAMITEDLLTGMRTAVPQIRVRSKILAERLGRAIGRETLPAMIHHEWAFVMGKILTF
MADNVGMNADTVEGVLSLSEVTRRWDIGNSVSAVFNPDGLTIRVENTGYIMTRETACMIG
DIHAQFAIQYLAAYLDEVIGTRTSLSPAELTSLKLWGLNVLKLLGRNGYEVIACMEPIGY
AVLMMGRDRSPDPYVNDTYLNSILSEFPVDSDARACVEALLTIYMSFGTPHKVSDAFGLF
RMLGHPMVDGADGIEKMRRLSKKVKIPDQSTAIDLGAIMAELFVRSFVKKHKRWPNCSIN
LPPRHPFHHARLCGYVPAETHPLNNTASWAAVEFNQEFEPPRQYNLADIIDDKSCSPNKH
ELYGAWMKSKTAGWQEQKKLILRWFTETMVKPSELLEEIDAHGFREEDKLIGLTPKEREL
KLTPRMFSLMTFKFRTYQVLTESMVADEILPHFPQITMTMSNHELTKRLISRTRPQSGGG
RDVHITVNIDFQKWNTNMRHGLVKHVFERLDNLFGFTNLIRRTHEYFQEAKYYLAEDGTN
LSFDRNGELIDGPYVYTGSYGGNEGLRQKPWTIVTVCGIYKVARDLKIKHQITGQGDNQV
VTLIFPDRELPSDPVERSKYCRDKSSQFLTRLSQYFAEVGLPVKTEETWMSSRLYAYGKR
MFLEGVPLKMFLKKIGRAFALSNEFVPSLEEDLARVWSATSAAVELDLTPYVGYVLGCCL
SAQAIRNHLIYSPVLEGPLLVKAYERKFINYDGGTKRGAMPGLRPTFESLVKSICWKPKA
IGGWPVLMLEDLIIKGFPDPATSALAQLKSMVPYTSGIDREIILSCLNLPLSSVVSPSML
LKDPAAINTITTPSAGDILQEVARDYVTDYPLQNPQLRAVVKNVKTELDTLASDLFKCEP
FFPPLMSDIFSASLPAYQDRIVRKCSTTSTIRRKAAERGSDSLLNRMKRNEINKMMLHLW
ATWGRSPLARLDTRCLTTCTKQLAQQYRNQSWGKQIHGVSVGHPLELFGRITPSHRCLHE
EDHGDFLQTFASEHVNQVDTDITTTLGPFYPYIGSETRERAVKVRKGVNYVVEPLLKPAV
RLLRAINWFIPEESDASHLLSNLLASVTDINPQDHYSSTEVGGGNAVHRYSCRLSDKLSR
VNNLYQLHTYLSVTTERLTKYSRGSKNTDAHFQSMMIYAQSRHIDLILESLHTGEMVPLE
CHHHIECNHCIEDIPDEPITGDPAWTEVKFPSSPQEPFLYIRQQDLPVKDKLEPVPRMNI
VRLAGLGPEAISELAHYFVAFRVIRASETDVDPNDVLSWTWLSRIDPDKLVEYIVHVFAS
LEWHHVLMSGVSVSVRDAFFKMLVSKRISETPLSSFYYLANLFVDPQTREALMSSKYGFS
PPAETVPNANAAAAEIRRCCANSAPSILESALHSREVVWMPGTNNYGDVVIWSHYIRLRF
SEVKLVDITRYQQWWRQSERDPYDLVPDMQVLESDLDTLMKRIPRLMRKARRPPLQVIRE
DLDVAVINADHPAHSVLQNKYRKLIFREPKIITGAVYKYLSLKSELTEFTSAMVIGDGTG
GITAAMMADGIDVWYQTLVNYDHVTQQGLSVQAPAALDLLRGAPSGRLLNPGRFASFGSD
LTDPRFTAYFDQYPPFKVDTLWSDAEGDFWDKPSKLNQYFENIIALRHRFVKTNGQLVVK
VYLTQDTATTIEAFRKKLSPCAIIVSLFSTEGSTECFVLSNLIAPDTPVDLEMVENIPKL
TSLVPQRTTVKCYSRRVACISKRWGLFRSPSIALEVQPFLHYITKVISDKGTQLSLMAVA
DTMINSYKKAISPRVFDLHRHRAALGFGRRSLHLIWGMIISPIAYQHFENPAKLMDVLDM
LTNNISAFLSISSSGFDLSFSVSADRDVRIDSKLVRLPLFEGSDLKFMKTIMSTLGSVFN
QVEPFKGIAINPSKLMTVKRTQELRYNNLIYTKDAILFPNEAAKNTAPLRANMVYPVRGD
LFAPTDRIPIMTLVSDETTPQHSPPEDEA
(Farmington rhabdovirus ORF1)
SEQ ID NO: 8
atggctcgtc cgctagctgc tgcgcaacat ctcataaccg agcgtcattc ccttcaggcg 60
actctgtcgc gggcgtccaa gaccagagcc gaggaattcg tcaaagattt ctaccttcaa 120
gagcagtatt ctgtcccgac catcccgacg gacgacattg cccagtctgg gcccatgctg 180
cttcaggcca tcctgagcga ggaatacaca aaggccactg acatagccca atccatcctc 240
tggaacactc ccacacccaa cgggctcctc agagagcatc tagatgccga tgggggaggc 300
tcattcacag cgctgcccgc gtctgcaatc agacccagcg acgaggcgaa tgcatgggcc 360
gctcgcatct ccgactcagg gttggggcct gtcttctatg cagccctcgc tgcttacatc 420
atcggctggt caggaagagg agagactagc cgcgtgcagc agaacatagg tcagaaatgg 480
ctgatgaacc tgaacgcaat cttcggcacc acgatcaccc atccaacaac cgtgcgtctg 540
ccaatcaacg tcgtcaacaa cagcctcgca gtgaggaacg gacttgctgc cacactctgg 600
ctatactacc gttcatcacc tcagagtcag gacgcgttct tctatgggct catccgtccc 660
tgttgcagtg gatatctcgg cctgctacat cgggtgcagg agattgatga gatggagccg 720
gacttcctca gtgacccccg gatcatccag gtgaatgagg tctacagtgc actcagagcc 780
ctggttcaac tgggaaacga cttcaagacc gccgatgatg agcccatgca ggtctgggcg 840
tgcaggggaa tcaacaacgg atatctgaca tatctctcag aaactcctgc gaagaaagga 900
gctgttgtgc ttatgtttgc ccaatgcatg ctgaagggcg actctgaggc ctggaacagc 960
taccgcactg caacctgggt gatgccctat tgcgacaatg tggccctagg agcgatggca 1020
ggctacatcc aagcccgcca gaacaccagg gcatatgagg tctcagccca gacaggtctc 1080
gacgtcaaca tggccgcggt caaggacttt gaggccagtt caaaacccaa ggctgctcca 1140
atctcgctga tcccacgccc cgctgatgtc gcatcccgca cctctgagcg cccatctatt 1200
cctgaggttg acagcgacga agagctcgga ggaatg 1236
(Farmington rhabdovirus ORF2)
SEQ ID NO: 9
atggaggact atttgtctag cttagaggcc gcgagagagc tcgtccggac ggagctggag 60
cccaagcgta acctcatagc cagcttagag tccgacgatc ccgatccggt aatagcgcca 120
gcggtaaaac caaaacatcc caagccatgc ctgagcacta aagaagagga tcatctcccc 180
tctcttcgcc tactattcgg cgcaaaacga gacacctcgg tgggcgtaga gcagactctc 240
cacaagcgtc tctgcgcttg tctcgacggt tacctgacca tgacgaagaa agaggccaat 300
gcctttaagg ccgcggctga agcagcagca ttagcagtca tggacattaa gatggagcat 360
cagcgccagg atctagagga tctgaccgct gctatcccta ggatagaatt caaactcaat 420
gccatcctgg aaaacaacaa ggagatagcc aaggctgtaa ctgctgctaa ggagatggag 480
cgggagatgt cgtgggggga aagcgccgcc agctcgctca agtctgtcac cctagatgag 540
tcgtttaggg gccctgaaga gctttcagag tcatttggca tccgatataa ggtcagaacc 600
tggaatgagt tcaagaaggc gctggaaacc agcattgtgg acctgaggcc tagccctgtt 660
tcatttaggg aattacggac tatgtggctg tctcttgaca cctcctttag gctcattggg 720
tttgccttca ttcccacatg cgagcgcctg gagaccaaag ccaaatgcaa ggagacaagg 780
actctactcc cccttgcaga gtcgatcatg cgaagatggg acctgcggga tccaaccatc 840
ttggagaaag cctgcgtagt aatgatgatc cgtgggaatg agattgcatc gctgaatcag 900
gtaaaagatg ttctcccgac cacaattcgt gggtggaaga tcgcttat 948
(Farmington rhabdovirus ORF3)
SEQ ID NO: 10
atgcgtcggt tctttttagg agagagcagt gcccctgcga gggactggga gtccgagcga 60
cctcccccct atgctgttga ggtccctcaa agtcacggga taagagtcac cgggtacttc 120
cagtgcaacg agcgtccgaa atccaagaag accctccaca gcttcgccgt aaaactctgc 180
gacgcaatta agccggttcg agcggatgct cccagcttga agatagcaat atggacggct 240
ctagatctgg ccttcgtgaa acctcccaat ggaactgtaa caatagatgc ggcggtgaaa 300
gctacaccgc taatcgggaa cacccagtac accgtaggcg atgaaatctt ccagatgcta 360
gggagaaggg gtggcctgat cgtcatcagg aacttacccc atgattatcc tcgaacgttg 420
attgagttcg cctctcccga gcct 444
(Farmington rhabdovirus ORF4)
SEQ ID NO: 11
atgctcagga tccagatccc tccgattgct atcattctgg taagtctcct cacactcgac 60
ctgtccggtg caaggaggac aaccacacaa agaatccctc tccttaatga ttcgtgggat 120
ttgttctcga gctatggcga cattcccgaa gaacttgtcg tataccagaa ctacagccac 180
aattcctccg agttaccccc tcctggcttc gagagatggt acataaaccg aagagtggca 240
gacacttcca taccgtgcag gggcccctgt ctagtgccct acatccttca tggcctcaat 300
gacacaactg tctctcgacg gggaggagga tggcgaaggt ccggaatgaa gtacccaacc 360
cacgctgtca ggctaggccc ttcaacagac gacgagagag ttgaggaaga catcggctac 420
gtcaatgtct ccgcactatc ctgcacaggg tcgcccgttg agatggcgat accaacaatc 480
cccgactgca ccagtgctat ccatccacga tccgaggtta ctgtgcccgt caagctcgat 540
gtcatgagac gaaatcccaa ctaccctccc attagagcgt ggtcgtgcat cggacagaaa 600
atcaccaacc gatgtgattg ggcactcttc ggcgagaacc tcatatatac tcaagttgaa 660
gctagctctc tagcattcaa gcacacaaga gcctctcttt tgaacgaatc caacgggata 720
gacgctgaag gacgtgcagt tccctatatc ctcggggata tcgaacccgg gtactgccga 780
accctattca acacatgggt ctctagtgag atcgtgtcat gcacgcccat cgaacttgtc 840
ctagttgacc tgaacccttt gtccccggga catggcggat atgctgtatt gctgccaaac 900
ggagacaaag tggatgtaca cgacaagcat gcatgggatg gggacaacaa aatgtggaga 960
tgggtgtacg agaagaaaga tccctgtgcg ttcgagctgg tatccaggga agtgtgtctt 1020
ttctcactga gtaggggtag tagactgaga ggagcaaccc ctccccaagg agagctcctc 1080
acctgcccgc attcgggaaa ggcatttgac ctgaaggggg cccgaaggat tacacccatt 1140
tcatgcaaaa tcgacatgga atatgacttg ctgtcactac caaccggagt catcctaggc 1200
ctccacctat cagaactcgg gacctccttt ggcaacctct caatgagtct tgaaatgtat 1260
gaacctgcca caactctgac ccctgagcaa atcaacttct cgcttaaaga gctgggaagc 1320
tggaccgagg ctcaactgaa gagcctgtct cactcaatct gcctctccac attctccata 1380
tgggaactat cggttgggat gatcgatcta aaccctacca gggcagcaag ggccttgctc 1440
catgatgata acatactggc aacattcgag aacggtcact tttccatcgt cagatgtcgt 1500
ccggaaatag ttcaagtccc ttcgcatcct cgagcatgtc acatggatct ccgcccttat 1560
gacaagcaat cacgggcatc aaccctggtg gttccccttg acaacagcac tgccctcctg 1620
gtccccgaca acatcgtggt tgaaggagta gaggccagtc tatgcaacca ctccgttgcc 1680
atcacgctgt cgaagaacag aactcactca tacagcctct atccccaggg tcgtcctgtg 1740
cttcgacaga aaggtgccgt ggagctcccg acgatagggc ccctccagtt acatcctgcc 1800
actcgagtgg acctttatac actgaaagag ttccaggagg accgaatagc gcgcagtcga 1860
gtcacagaca tcaaggctgc cgttgacgat ctgcgtgcga agtggcgtaa aggcaaattt 1920
gaggcggaca ccacgggagg gggactttgg tcggcgattg tgggagtctt cagttctctc 1980
ggggggttct tcatgaggcc cttgattgct ctcgcggcga tagtgacctc aatcatcatc 2040
ctgtatatcc ttctgcgtgt actgtgtgct gcctcatgtt cgacacaccg aagagtaagg 2100
caggactctt gg 2112
(Farmington rhabdovirus ORF5)
SEQ ID NO: 12
atggccttcg acccgaactg gcagagagaa ggttatgaat gggatccgtc aagtgagggc 60
agaccgaccg atgagaacga agacgacaga ggtcatcggc caaaaacgag acttcgtaca 120
ttccttgccc gcacgttaaa tagccctatc cgagccctat tctacacaat attcctagga 180
attcgagcgg tttgggacgg gttcaaaaga ctcctacctg tgaggaccga aaagggttat 240
gcgaggtttt ctgagtgcgt cacatatgga atgatcggat gtgatgagtg tgtaatagac 300
ccggtgaggg ttgtcattga gctgaccgag atgcagttac cgattaaagg caaaggctct 360
acgaggttga gagcaatgat aactgaagac cttctcacgg ggatgcgcac agccgtgcct 420
cagatcagag tgagatcgaa gatcctagca gagcggttag ggagagcaat cggccgagag 480
accttgccgg caatgatcca tcatgagtgg gcatttgtga tggggaagat tctcactttc 540
atggcagaca atgtgggtat gaacgctgac acggtcgagg gcgttctatc actatcagag 600
gtcacacggc gatgggatat cggcaactct gtgtccgcag tgttcaatcc tgatggcctt 660
actatcagag tagaaaacac gggttacatc atgaccagag agactgcctg catgatcgga 720
gacattcatg ctcaatttgc aatccaatac ctagctgcat acctagacga ggtgatcggc 780
acaaggacgt ctctctcacc cgccgaactg acctctctca aactatgggg acttaacgtc 840
ctgaaactcc taggacggaa cggttatgag gtgatcgcct gcatggagcc catagggtac 900
gctgtcctga tgatgggaag agacaggagt cctgatccct atgtcaatga cacctattta 960
aacagcatcc tctcagaatt ccctgtcgac tctgacgctc gagcctgcgt tgaagccctc 1020
ttaactatct atatgagctt cggcacaccc cataaagtct cggacgcatt cggcctcttc 1080
agaatgttgg gacatccgat ggttgatgga gctgacggga ttgaaaagat gcgaaggtta 1140
agcaagaagg tcaagatccc agaccagtct acagcgatcg acctcggggc tatcatggcc 1200
gaactgtttg tgcggagttt cgtaaagaag cacaaaaggt ggcccaactg ctccatcaat 1260
ctcccgccac gacacccctt ccaccacgcc cgcctatgtg ggtatgtccc ggctgaaacc 1320
catcccctaa acaacactgc atcctgggcg gctgtggagt tcaaccagga attcgagccg 1380
ccgagacagt acaaccttgc agacatcatt gatgacaagt cgtgctctcc caacaagcat 1440
gagctatatg gtgcttggat gaagtcaaaa acagctgggt ggcaggaaca aaagaagctc 1500
atactccgat ggttcactga gaccatggtt aaaccttcgg agctcctgga agagattgat 1560
gcacacggct tccgagaaga ggataagttg attggattaa caccaaagga gagagagctg 1620
aaattaacac caagaatgtt ctccttgatg acattcaagt tcagaaccta ccaagtcctc 1680
actgagagta tggtcgccga tgagatcctc ccgcacttcc cccagatcac catgaccatg 1740
tccaaccacg aactcacaaa gaggttgatt agcagaacga gacctcaatc tggaggaggg 1800
cgtgatgttc acatcaccgt gaacatagat ttccagaaat ggaacacaaa catgagacac 1860
ggactggtca aacatgtctt cgagcgactg gacaacctct ttggcttcac caacttaatc 1920
agacgaactc atgaatactt ccaggaggcg aaatactatc tggctgaaga tggaactaat 1980
ctgtcgttcg acaggaacgg ggagttaata gatggcccat acgtttacac cggatcatac 2040
ggggggaacg aggggttacg acagaagccc tggacaatag ttaccgtgtg tggaatatac 2100
aaggtagcta gagacctgaa aatcaaacat cagatcaccg gtcagggaga taatcaggtg 2160
gtcaccctaa tatttccgga tcgagagttg ccttcagatc cggtggagag gagcaagtac 2220
tgtagagaca agagcagtca gttcctgaca cgtctcagtc aatatttcgc tgaggttggt 2280
ttgcccgtca agactgaaga gacatggatg tcatcacgtc tctatgctta cggtaagcgc 2340
atgttcttag agggagttcc acttaagatg tttctcaaga agataggcag agctttcgcc 2400
ctctcgaatg agtttgtccc gtccctcgag gaagatctgg ccagagtctg gagtgccacc 2460
agcgcagcgg tagagcttga cctaactccc tacgtaggat atgtcctcgg gtgctgcttg 2520
tctgcgcagg cgatcagaaa tcacctcatc tactcccctg ttctggaggg ccctctgctg 2580
gttaaggcct acgagcgtaa gttcattaac tacgacggag gaacaaagcg gggggcgatg 2640
cccggcctac gtccaacctt tgagagccta gtcaaaagta tctgctggaa gccaaaggcc 2700
atcggagggt ggccggtatt gatgttagaa gatctcatca tcaaagggtt ccctgatccg 2760
gcgactagcg ccctggctca attgaagtca atggtgccat atacctctgg tatcgaccgg 2820
gagatcatac tttcctgtct caaccttccc ttatcgtcgg tggtatctcc gtcaatgttg 2880
ttaaaggacc cggcggccat caacaccatc acaaccccgt ccgcgggcga catcctgcaa 2940
gaggtcgcca gagactatgt taccgattac ccactccaaa acccgcagct cagagcagtg 3000
gtcaagaacg tgaagaccga gctagacaca ttggccagtg acttattcaa atgtgaacct 3060
ttctttcctc ctttaatgag cgatatcttc tcggcatctc tcccggcata tcaagacagg 3120
attgttcgca agtgctccac gacttctaca atcaggagaa aagctgccga gaggggctcc 3180
gactctctcc tcaaccggat gaaaaggaat gagatcaata agatgatgtt acatctttgg 3240
gctacctggg gaaggagccc tctggccaga ttagacacca gatgtctcac aacctgcacc 3300
aagcaattag cccaacagta tcggaaccag tcttggggaa agcagatcca tggagtctca 3360
gtcggccacc ccttagaact gttcggtcga ataacaccca gccatagatg cctacatgag 3420
gaggaccacg gagatttcct gcaaaccttc gccagcgagc atgtgaacca agtggacacc 3480
gacatcacca caactctggg gccgttctac ccttacatag gctcggagac gcgagaacgg 3540
gcagtcaagg ttcgaaaagg agtgaattac gtagttgagc cgcttctgaa acccgcagtt 3600
cgactactaa gagccattaa ttggttcatt cccgaggagt cagatgcgtc ccatttgctg 3660
agcaatctat tagcgtctgt taccgacatc aatcctcaag accactactc atctaccgaa 3720
gtaggggggg gcaacgccgt ccatcgctac agctgccgac tatccgacaa attgagcaga 3780
gtcaacaact tatatcagtt gcatacttat ttatctgtca caacagagcg gttgaccaag 3840
tacagtcgag gatcaaaaaa cactgacgca cacttccaga gcatgatgat ttatgcacaa 3900
agccgtcata tagacctcat cttggagtct ctgcacaccg gagagatggt accgttggag 3960
tgtcatcatc acattgagtg caatcactgt atagaggata tacccgacga gccaatcacg 4020
ggggacccgg cttggactga agtcaagttt ccttcaagtc ctcaggagcc ctttctttac 4080
atcaggcaac aagatctgcc ggtcaaagac aaactcgagc ctgtgcctcg catgaacatc 4140
gtccgtcttg ccggattggg tccggaggcg attagtgagc tagcgcacta ctttgttgca 4200
ttccgagtta tccgggcgtc agagacggat gtcgacccta acgatgttct ctcgtggacc 4260
tggctgagcc gaattgatcc tgacaaattg gttgagtata tcgtgcatgt gttcgcttca 4320
ctggaatggc atcatgtatt aatgtcaggc gtgagtgtga gcgtcagaga tgcattcttt 4380
aagatgctag tgtctaaaag aatctcagag actccgctaa gttcattcta ttatctggcc 4440
aacctgttcg ttgaccctca gactcgcgaa gcactaatga gctctaaata cgggttcagc 4500
ccccccgccg agacagtccc caacgcaaat gccgccgcag ccgaaataag aagatgctgt 4560
gcgaacagtg cgccgtcgat cttagaatca gcccttcaca gccgtgaggt tgtttggatg 4620
ccaggaacga acaattatgg agacgttgtc atctggtctc attacattag attacggttc 4680
agcgaagtta aactagttga cattacacga tatcagcagt ggtggagaca gtctgagcga 4740
gacccctacg atttggtccc ggacatgcag gttcttgaga gcgacctaga tacgctgatg 4800
aaacggatac cgaggctcat gcgcaaggcg agacgtcccc ctcttcaggt aattcgagag 4860
gacctggatg tcgcagtcat caatgctgat catcccgctc actctgtgct tcagaacaaa 4920
tacaggaaat tgattttcag agagccgaag attatcacgg gagctgtgta caagtacctc 4980
tccctaaaat cagagttgac agagttcacc tcagcaatgg tgatcggaga cggaactgga 5040
ggtatcaccg ccgccatgat ggccgatggg atagatgtgt ggtatcagac gctcgtcaac 5100
tatgaccacg tgacacaaca gggattatcc gtacaagccc cggcagcatt ggatcttctg 5160
cgcggggcac cctctggtag gctcttgaat ccgggaagat tcgcatcatt tgggtctgac 5220
ctaactgacc ctcgatttac agcctacttt gatcaatatc ccccgttcaa ggtggacact 5280
ctatggtctg acgcagaggg cgacttttgg gacaagcctt ccaagttgaa tcaatacttt 5340
gagaacatca ttgctttgag acatcggttc gtgaagacaa atggacagct tgtcgtgaag 5400
gtgtatctga ctcaagacac tgctaccaca attgaagcat tcagaaagaa gctgtcccca 5460
tgcgccatca tcgtgtctct cttctcgacg gaaggctcca cagaatgctt cgtcctaagc 5520
aatctcatcg caccagacac ccctgtcgac cttgagatgg tggagaatat ccctaaacta 5580
acatcccttg ttccccagag gacgacagtg aaatgctatt cccgacgagt agcgtgcatc 5640
agtaaaaggt ggggactttt cagatctccg agcatagccc ttgaagtcca accgttcctt 5700
cactacatca caaaggtcat ctcagacaaa ggaacacaac tgagtctcat ggcggtagct 5760
gacacaatga tcaacagtta caagaaggct atctcacccc gagtgttcga tctacaccgg 5820
catagggccg cactgggttt cgggaggaga tccttgcatc tcatctgggg gatgatcatc 5880
tcaccaatcg cttaccagca ttttgagaat ccggccaagt tgatggatgt cctggacatg 5940
ttgaccaata acatctcagc tttcttatcg atatcgtcgt caggatttga cctgtcattt 6000
agtgtcagtg cagaccgaga tgtccggatt gacagcaaac ttgtcagact cccgctattc 6060
gaaggatcag acctaaaatt catgaaaacc atcatgtcta ccctcggatc tgtgttcaac 6120
caggtcgagc cttttaaggg gatcgccata aacccttcta aactaatgac tgtcaagagg 6180
acacaggagt tacgttacaa caacctaatt tacactaagg atgccatcct attccccaat 6240
gaagcggcaa aaaacactgc cccgcttcga gccaacatgg tataccccgt ccggggagat 6300
ctattcgccc ctaccgatcg cataccaatc atgactctag tcagcgatga gacaacacct 6360
cagcactctc ctccagagga tgaggca 6387
(Protein sequence of full length, wild type, human MAGEA3)
SEQ ID NO: 13
MPLEQRSQHCKPEEGLEARGEALGLVGAQAPATEEQEAASSSSTLVEVTLGEVPAAESPDPPQSPQGASSLP
TTMNYPLWSQSYEDSSNQEEEGPSTFPDLESEFQAALSRKVAELVHFLLLKYRAREPVTKAEMLGSWGNWQY
FFPVIFSKASSSLQLVFGIELMEVDPIGHLYIFATCLGLSYDGLLGDNQIMPKAGLLIIVLAIIAREGDCAP
EEKIWEELSVLEVFEGREDSILGDPKKLLTQHFVQENYLEYRQVPGSDPACYEFLWGPRALVETSYVKVLHH
MVKISGGPHISYPPLHEWVLREGEE
(Protein sequence of a variant of full length, wild type,
human MAGEA3)
SEQ ID NO: 14
MPLEQRSQHCKPEEGLEARGEALGLVGAQAPATEEQEAASSSSTLVEVTLGEVPAAESPDPPQSPQGASSLP
TTMNYPLWSQSYEDSSNQEEEGPSTFPDLESEFQAALSRKVAELVHFLLLKYRAREPVTKAEMLGSWGNWQY
FFPVIFSKASSSLQLVFGIELMEVDPIGHLYIFATCLGLSYDGLLGDNQIMPKAGLLIIVLAIIAREGDCAP
EEKIWEELSVLEVFEGREDSILGDPKKLLTQHFVQENYLEYRQVPGSDPACYEFLWGPRALVETSYVKVLHH
MVKISGGPHISYPPLHEWVLREGEEDYKDDDDK*
(artificial HPV16 E6 protein sequence)
Each X can be present or absent; if present, X can be any naturally
occuring amino acid. When all X's are cysteines, the sequence
corresponds to the wildtype HPV16 E6 protein sequence.
SEQ ID NO: 15
MHQKRTAMFQDPQERPRKLPQLCTELQTTIHDIILEXVYXKQQLLRREVYDFAFRDLCIV
YRDGNPYAVXDKXLKFYSKISEYRHYCYSLYGTTLEQQYNKPLCDLLIRXINXQKPLCPE
EKQRHLDKKQRFHNIRGRWTGRXMSXCRSSRTRRETQL
(artificial HPV18 E6 protein sequence)
Each X can be present or absent; if present, X can be any naturally
occuring amino acid. When all X's are cysteines, the sequence
corresponds to the wildtype HPV18 E6 protein sequence.
SEQ ID NO: 16
MARFEDPTRRPYKLPDLCTELNTSLQDIElTXVYXKTVLELTEVFEFAFKDLFVVYRDSI
PHAAXHKXIDFYSRIRELRHYSDSVYGDTLEKLTNTGLYNLLIRXLRXQKPLNPAEKLRH
LNEKRRFHNIAGHYRGQXHSXCNRARQERLQRRRETQV
(artificial HPV16 E7 protein sequence)
Each X can be present or absent; if present, X can be any naturally
occuring amino acid. When XXX is CYE and X's at positions 91 and 94
are cysteine, the sequence corresponds to the wildtype HPV16 E7
protein sequence.
SEQ ID NO: 17
MHGDTPTLHEYMLDLQPETTDLYXXXQLNDSSEEEDEIDGPAGQAEPDRAHYNIVTFCCK
CDSTLRLCVQSTHVDIRTLEDLLMGTLGIVXPIXSQKP
(artificial HPV18 E7 protein sequence)
Each X can be present or absent; if present, X can be any naturally
occuring amino acid. When XXX is CHE and X's at positions 98 and 101
are cysteine, the sequence corresponds to the wildtype HPV18 E7
protein sequence.
SEQ ID NO: 18
MHGPKATLQDIVLHLEPQNEIPVDLLXXXQLSDSEEENDEIDGVNHQHLPARRAEPQRHT
MLCMCCKCEARIKLVVESSADDLRAFQQLFLNTLSFVXPWXASQQ
(codon-optimized human STEAP protein)
SEQ ID NO: 19
MESRKDITNQEELWKMKPRRNLEEDDYLHKDTGETSMLKRPVLLHLHQTAHADEFDCPSE
LQHTQELFPQWHLPIKIAAIIASLTFLYTLLREVIHPLATSHQQYFYKIPILVINKVLPM
VSITLLALVYLPGVIAAIVQLHNGTKYKKFPHWLDKWMLTRKQFGLLSFFFAVLHAIYSL
SYPMRRSYRYKLLNWAYQQVQQNKEDAWIEHDVWRMEIYVSLGIVGLAILALLAVTSIPS
VSDSLTWREFHYIQSKLGIVSLLLGTIHALIFAWNKWIDIKQFVWYTPPTFMIAVFLPly
VLIFKSILFLPCLRKKILKIRHGWEDVTKINKTEICSQLKL
(Protein sequence of NYESQ1 MAR protein)
SEQ ID NO: 20
MQAEGRGTGGSTGDADGPGGPGIPDGPGGNAGGPGEAGATGGRGPRGAGAARASGPGGGAPRGPHGGAASGL
NGCCRCGARGPESRLLEFYLAMPFATPMEAELARRSLAQDAPPLPVPGVLLKEFTVSGNILTIRLTAADHRQ
LQLSISSCLQQLSLLMWITQCFLPVFLAQPPSGQRR*
(Isoform 1 of human Brachyury protein; Uniprot database under
identifier O15178-1)
SEQ ID NO: 21
MSSPGTESAGKSLQYRVDHLLSAVENELQAGSEKGDPTERELRVGLEESE
LWLRFKELTNEMIVTKNGRRMFPVLKVNVSGLDPNAMYSFLLDFVAADNH
RWKYVNGEWVPGGKPEPQAPSCVYIHPDSPNFGAHWMKAPVSFSKVKLTN
KLNGGGQIMLNSLHKYEPRIHIVRVGGPQRMITSHCFPETQFIAVTAYQN
EEITALKIKYNPFAKAFLDAKERSDHKEMMEEPGDSQQPGYSQWGWLLPG
TSTLCPPANPHPQFGGALSLPSTHSCDRYPTLRSHRSSPYPSPYAHRNNS
PTYSDNSPACLSMLQSHDNWSSLGMPAHPSMLPVSHNASPPTSSSQYPSL
WSVSNGAVTPGSQAAAVSNGLGAQFFRGSPAHYTPLTHPVSAPSSSGSPL
YEGAAAATDIVDSQYDAAAQGRLIASWTPVSPPSM
(Isoform 1 of human prostatic acid phosphatase; Uniprot database
under identifier P15309-1)
SEQ ID NO: 22
MRAAPLLLARAASLSLGFLELLFEWLDRSVLAKELKEVTLVERHGDRSPI
DTFPTDPIKESSWPQGFGQLTQLGMEQHYELGEYIRKRYRKFLNESYKHE
QVYIRSTDVDRTLMSAMTNLAALFPPEGVSIWNPILLWQPIPVHTVPLSE
DQLLYLPFRNCPRFQELESETLKSEEFQKRLHPYKDFIATLGKLSGLHGQ
DLFGIWSKVYDPLYCESVHNFTLPSWATEDTMTKLRELSELSLLSLYGIH
KQKEKSRLQGGVLVNEILNHMKRATQIPSYKKLIMYSAHDTTVSGLQMAL
DVYNGLLPPYASCHLTELYFEKGEYFVEMYYRNETQHEPYPLMLPGCSPS
CPLERFAELVGPVIPQDWSTECMTTNSHQGTEDSTD
(tumour associated epitope)
SEQ ID NO: 23
EVDPIGHLY
(tumour associated epitope)
SEQ ID NO: 24
FLWGPRALV
(tumour associated epitope)
SEQ ID NO: 25
KVAELVHFL
(tumour associated epitope)
SEQ ID NO: 26
TFPDLESEF
(tumour associated epitope)
SEQ ID NO: 27
VAELVHFLL
(tumour associated epitope)
SEQ ID NO: 28
REPVTKAEML
(tumour associated epitope)
SEQ ID NO: 29
AELVHFLLL
(tumour associated epitope)
SEQ ID NO: 30
WQYFFPVIF
(tumour associated epitope)
SEQ ID NO: 31
EGDCAPEEK
(tumour associated epitope)
SEQ ID NO: 32
KKLLTQHFVQENYLEY
(tumour associated epitope)
SEQ ID NO: 33
VIFSKASSSLQL
(tumour associated epitope)
SEQ ID NO: 34
VFGIELMEVDPIGHL
(tumour associated epitope)
SEQ ID NO: 35
GDNQIMPKAGLLIIV
(tumour associated epitope)
SEQ ID NO: 36
TSYVKVLHHMVKISG
(tumour associated epitope)
SEQ ID NO: 37
FLLLKYRAREPVTKAE

### EXPERIMENTS

In the following examples, it should be understood that the tested primes and the tested antigenic proteins provide proof of the concept that Farmington (FMT) virus may be used to generate an immune response in prime:boost combination treatments with different primes and with different classes of antigenic peptides. As demonstrated herein, the FMT virus may provide a boost of an immune response for a variety of types of primes and antigenic peptides.

### Experiment 1. FMT Virus Engineered to Express an Antigenic Protein Boosts Antigen-Specific Immune Responses in Three Different Prime Strategies

To characterize the FMT virus as a boost component in a combination prime: boost therapy, the authors of the present disclosure investigated the capacity of an FMT virus engineered to express mCMV-derived antigen m38 (FMT-m38) to expand m38-specific CD8 T cells in vivo when combined with three different primes:

1) Adenovirus (AdV) engineered to express m38 (AdV-m38),

2) adoptive cell transfer (ACT) of m38-specific CD8 memory T cells (ACT-m38) and

3) m38 peptide with adjuvant (peptide m38).

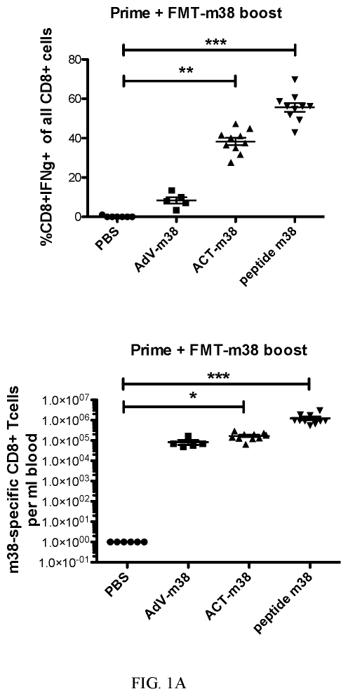

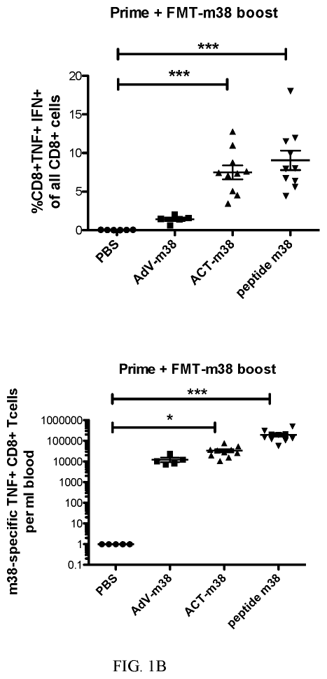

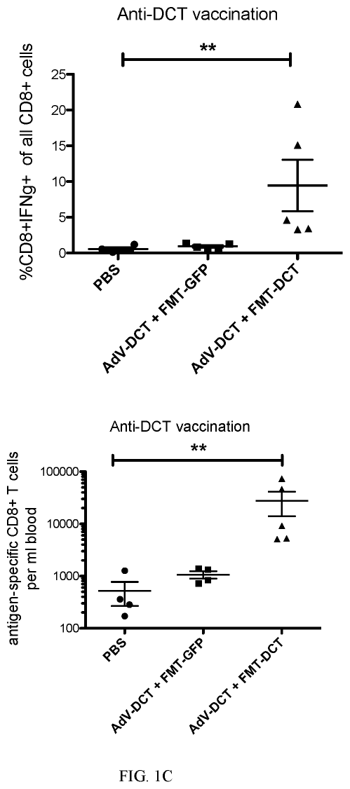

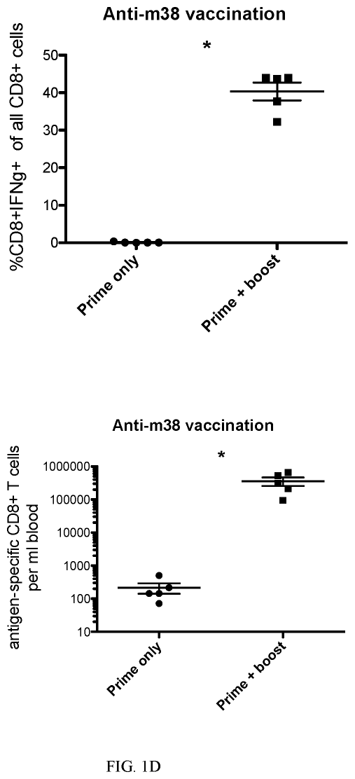

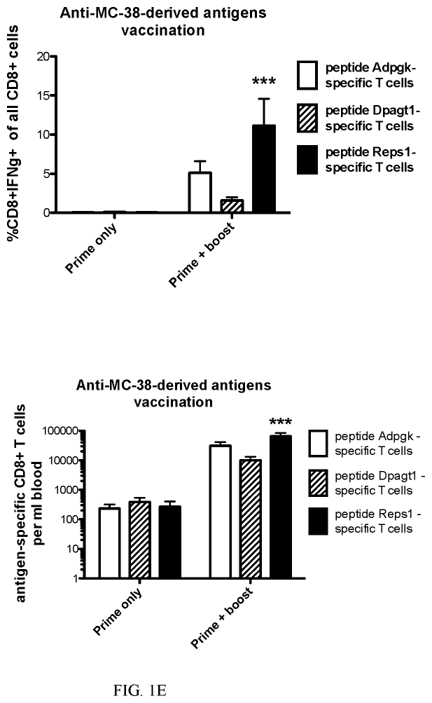

In each of these combinations FMT-m38 induced an increase in the frequencies (mean of 8.4%, 38.3% and 55.7% of all CD8 T cells for AdV-m38, ACT-m38 and m38 peptide prime, respectively, compared to 0.2% for PBS control, P<0.0001; See FIG. 1A) and numbers (mean of 8.2×10⁴, 16.8×10⁴ and 125.7×10⁴ cells for AdV-m38, ACT-m38 and m38 peptide prime, respectively, compared to 1 cell for PBS control, P<0.0001; see FIG. 1A) of m38-specific CD8 T cells defined as CD8 T cells expressing IFNγ upon ex-vivo stimulation with the dominant epitope of m38 antigen.

The same results were observed for poly-functional CD8 T cells expressing both IFNγ and TNFα upon peptide stimulation, although not all CD8+ IFN+ T cells secreted TNFα (FIG. 1B). Additionally, during the same assay but in separate wells the authors of the present disclosure assessed the CD8 immune response against the dominant epitope of the FMT virus. The frequencies of FMT-specific CD8 T cells in the ACT-m38-primed group were significantly higher compared to PBS (mean 1.1% vs 0.02%, P<0.001), but did not exceed 3% of all CD8 T cells, while the groups primed with AdV-m38 and m38 peptide were no different than PBS control (mean 0.06% and 0.13%, respectively, FIG. 8). These levels of FMT-specific CD8 T cells were consistent during all further experiments in naïve and tumour-bearing mice receiving FMT-m38 virus. To summarize, the authors of the present disclosure found that FMT virus can successfully be used as a boost in a variety of prime:boost treatment strategies with small or even hardly detectable levels of FMT-specific cellular immune responses.

### Experiment 2. FMT Virus-Based Prime:Boost Treatment Induces Potent Immune Responses Against Different Classes of Antigens

Even though some types of cancers express foreign antigens (for example glioblastomas expressing CMV proteins in CMV-positive patients), in most cases cancer vaccines need to target aberrantly expressed self-antigens or cancer-specific mutations manifested by neo-epitopes presented by MHC I.

The authors of the present disclosure tested FMT virus for its ability to act as a boost against three different classes of antigens:

1) tumour associated self-antigens,

2) foreign antigens and

3) tumour-derived neo-epitopes.

A prime:boost treatment directed against DCT, a melanoma-associated self-antigen, with AdV and FMT virus expressing DCT (AdV-DCT and FMT-DCT) as a prime and boost, respectively, resulted in an expansion of DCT-specific CD8 T cells compared to group primed with AdV-DCT and boosted with FMT virus with GFP encoded instead of DCT (FMT-GFP) and PBS control (mean frequency 9.4% of all CD8 T cells vs 0.9% and 0.6% for control groups, P=0.0070, mean number 2.8×10⁴ cells vs 0.1×10⁴ cells and 0.05×10⁴ cells for control groups, P=0.0076; see FIG. 1C). Immunization against m38, a mCMV-derived (foreign) antigen with ACT-m38 and FMT-m38 as prime and boost, respectively, induced high magnitude increase in m38-specific CD8 T cells frequencies (mean 40.3% vs 0.1%, P=0.0119; see FIG. 1D) and numbers (mean 3.6×10⁵ cells vs 0.002×10⁵ cells, P=0.0119; see FIG. 1D) compared with group that received only prime.

Next, the authors of the present disclosure assessed the ability of FMT virus to boost immune response against tumour-derived neo-epitopes. The authors of the present disclosure generated FMT virus expressing Adpgk, Dpagt1 and Reps1 (FMT-MC-38)—neo-epitopes derived from MC-38 murine colon carcinoma cell line and used it in combination with peptide-based prime. Importantly, this FMT-MC-38 virus expressed only the peptide fragments that constitute the CD8 T cell epitopes, not the whole antigens as FMT-DCT and FMT-m38. Compared to control group that received only prime, prime combined with FMT-MC-38 boost elevated the frequencies and numbers of CD8 T cells specific for each peptide (FIG. 1E): Adpgk (mean frequency 5.1% vs 0.06%, mean number 3.1×10⁴ cells vs 0.02×10⁴ cells, P>0.05), Dpagt1 (mean frequency 1.6% vs 0.09%, mean number 1×10⁴ cells vs 0.04×10⁴ cells, P>0.05) and Reps1 (mean frequency 11.1% vs 0.06%, mean number 6.5×10⁴ cells vs 0.03×10⁴ cells, P<0.001).

This demonstrates that FMT virus can be applied for immunization against different classes of antigens. Moreover, it is feasible to use engineered FMT virus for immune stimulation against one or more epitopes of interest without the necessity of expressing the whole antigen(s).

### Experiment 3. Immune Response Induced by an FMT Virus Boost can be Sustained Over Prolonged Periods of Time

The numbers of antigen-specific effector T cells contract within days following antigen stimulation, remaining a small pool of memory T cells that upon re-stimulation with the same antigen expand in numbers and differentiate to perform effector functions. Therefore, the authors of the present disclosure examined whether the immune response induced by a boosting Farmington virus according to the present disclosure can be re-stimulated again following the contraction phase and using the same boost.

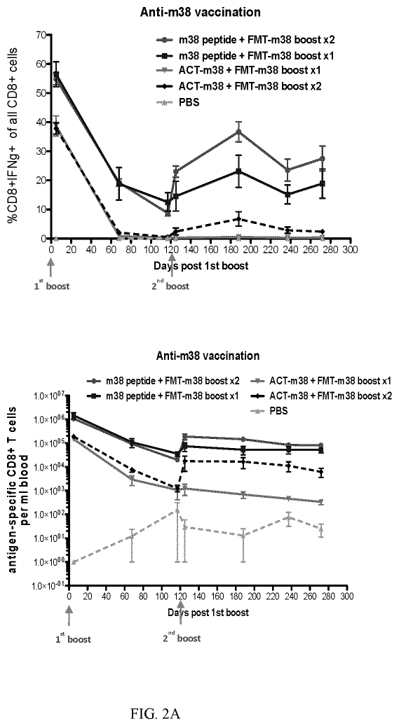

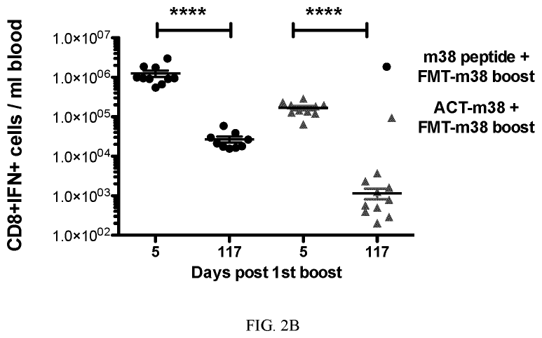

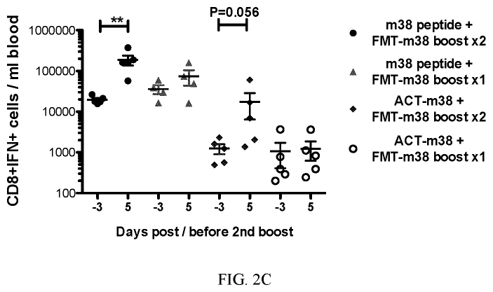

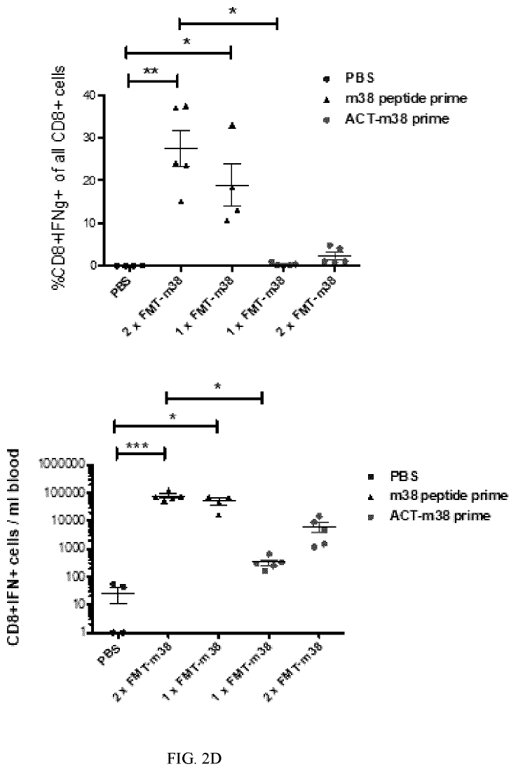

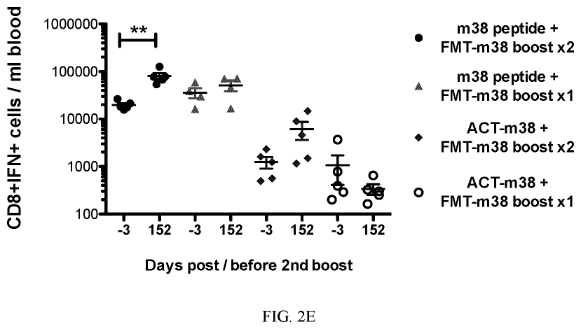

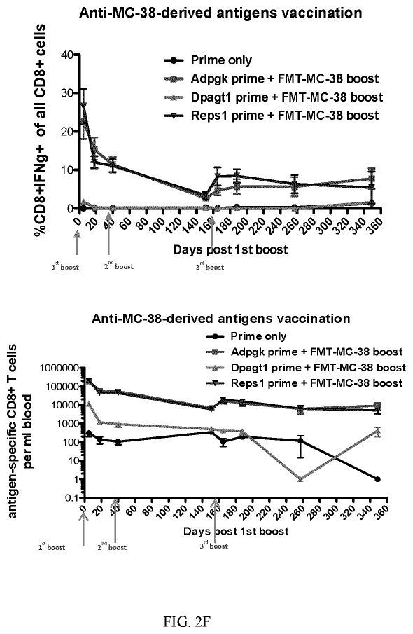

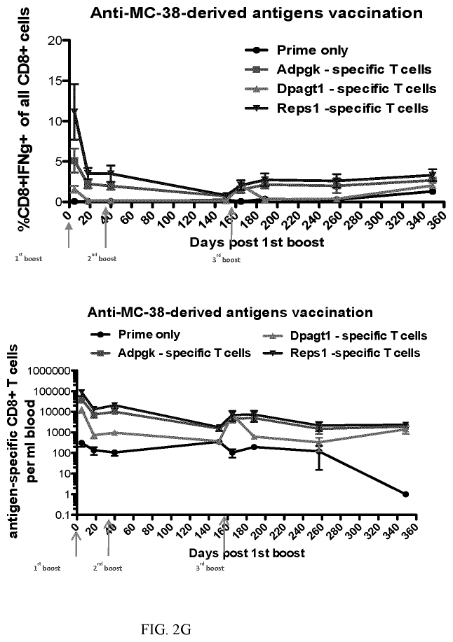

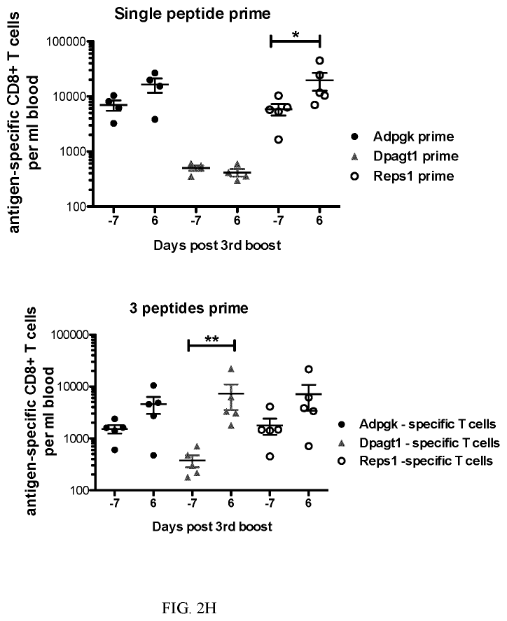

To address this, the authors of the present disclosure immunized mice against m38 antigen using FMT-m38 virus combined with ACT-m38 or m38 peptide prime and waited 120 days before boosting them again with FMT-m38 to minimize the risk of the virus being cleared by neutralizing antibodies before inducing any effect. As observed in the previous experiments, the first boost with FMT-m38 induced high m38-specific immune responses (see FIG. 2A, time point 5 days). The frequencies and numbers contracted within 112 days by over 95% in both ACT-m38- and m38 peptide-primed groups (from 1.7×10⁵ cells to 0.012×10⁵ cells in ACT-m38-primed mice, P<0.0001 and from 1.257×10⁶ cells to 0.027×10⁶ cells in m38 peptide-primed mice, P<0.0001; see FIG. 2A, 2B).

Each treatment group was then divided into mice receiving FMT-m38 for the second time and mice receiving PBS instead. Second boost with FMT-m38, but not PBS, resulted in an expansion of frequencies and numbers of m38-specific CD8 T cells compared to the residual pool before the second boost (in m38 primed mice: 1.9×10⁵ vs 0.2×10⁵ cells, P=0.0079 for FMT-m38 2nd boost and 7.4×10⁴ vs 3.6×10⁴ cells, P=0.49 for PBS 2nd boost control; in ACT-m38 primed mice 1.8×10⁴ vs 0.1×10⁴ cells, P=0.056 for FMT-m38 2nd boost and 1238 vs 1066 cells, P=0.60 for PBS 2nd boost control, FIG. 2C).

Surprisingly, even though the m38-specific CD8 T cell response underwent slow contraction (as evident by numbers of CD8+ IFN+ cells (FIG. 2A)), the difference between early and late time point post 2nd boost (5 vs 152 days) was not statistically significant and both the frequencies and amounts of m38-specific CD8 T cells in the m38 peptide primed mice were still significantly higher than in the PBS control, even in the group that received only one boost (FIG. 2A, D) and higher compared to before 2nd boost for mice primed with m38-peptide and boosted twice with FMT-m38 (FIG. 2E).

To further confirm the observations described above, the authors of the present disclosure immunostimulated mice against three MC-38-derived neo-epitopes: Adpgk, Dpagt1 and Reps1. Mice were primed with either all 3 long mutant peptides or with each peptide separately and all were boosted with FMT-MC-38 virus. For control, mice were primed with all 3 peptides and boosted with PBS (prime only control). Each immunostimulation expanded the frequencies and numbers of CD8 T cells specific to each epitope compared to prime only group (FIG. 2F, 2G, time point 5 days). The authors of the present disclosure first attempted to reduce the time interval between boosts and thus applied second FMT-MC-38 boost 35 days after the first boost while the immune response was still undergoing contraction (FIG. 2F, 2G). However, no expansion of antigen-specific CD8 T cells was detected (FIG. 2F, 2G). Therefore, the authors of the present disclosure repeated the boost 124 days later to resemble the time interval applied previously in anti-m38 immunostimulation experiment. The third boost with FMT-MC-38 resulted in the increased frequencies and numbers of CD8 T cells specific to each epitope in each treatment group, except Dpagt1 prime group, compared to measurement taken a week before 3rd boost, however, the difference was statistically significant only in Reps1 prime group (P=0.0159) and 3 peptides prime group for Dpagt1-specific CD8 T cells (P=0.0079) (mean cell numbers after vs before boost in mice primed with single peptides: 1.6×10⁴ vs 0.7×10⁴, 414 vs 500, and 2.0×10⁴ vs 0.6×10⁴ of Adpgk-, Dpagt1- and Reps1-specific CD8 T cells, respectively; and in mice primes with all 3 peptides: 4621 vs 1524, 7268 vs 374, and 7126 vs 1785 of Adpgk-, Dpagt1- and Reps1-specific CD8 T cells, respectively (FIG. 2H)). As in previous experiment, the immune response was sustained over long period of time as illustrated by antigen-specific CD8 T cell numbers at 190 days post 3rd boost compared to prime only control (FIG. 2I), however, at this time point as well as 98 days post 3rd boost it was at the same level as before 3rd boost.

The authors of the present disclosure thus conclude that FMT-based boost has the ability to induce long-lasting antigen-specific immune responses. It is also feasible to re-stimulate the CD8 T cells in a homologous setting provided long time interval (min. 120 days in mice) is applied between the boosts. Importantly, this can be achieved for both foreign antigen and neo-epitopes, and when boosted against whole antigen or one or more epitopes.

### Experiment 4. Treatment with an Exemplary Prime:Boost Therapy According to the Present Disclosure Improves Animals' Survival

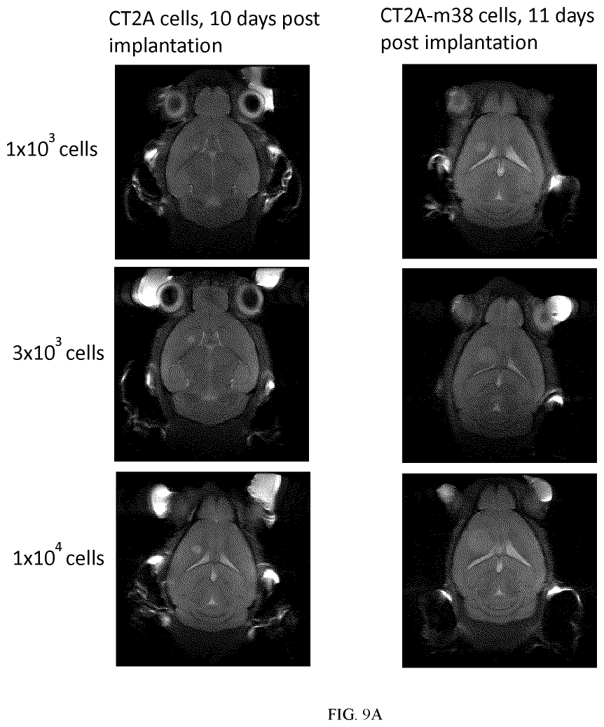

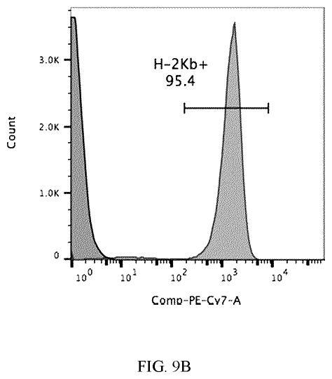

In order to determine the anti-tumour efficacy of FMT-based prime:boost treatment in vivo, the authors of the present disclosure treated tumour-bearing immunocompetent mice with a prime:boost therapy. First the authors focused on targeting CMV antigen in glioma mouse model, as the safety profile of FMT virus makes it a particularly promising tool for targeting brain tumours. For this purpose, the authors engineered murine glioma CT2A cells to express m38 antigen and generated a stable CT2A-m38 cell line. Tumour cells extracted from mice 21 days after intracranial implantation of CT2A-m38 cells expressed major histocompatibility complex class I (MHC I) allele that presents the m38 epitope (FIG. 9B).

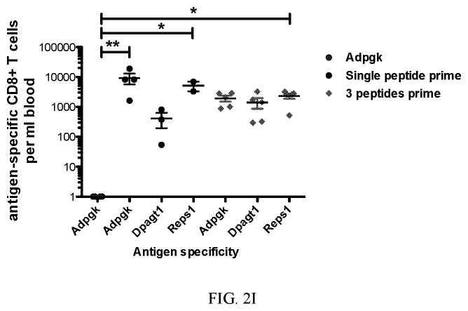

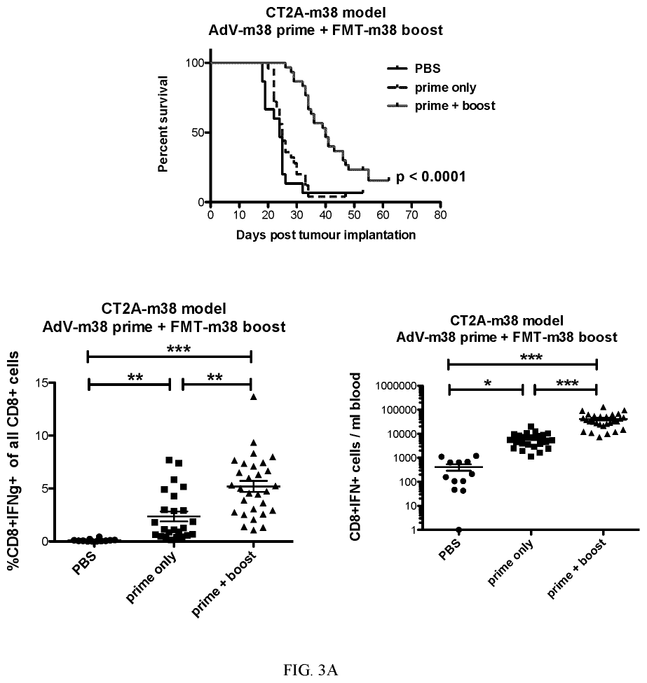

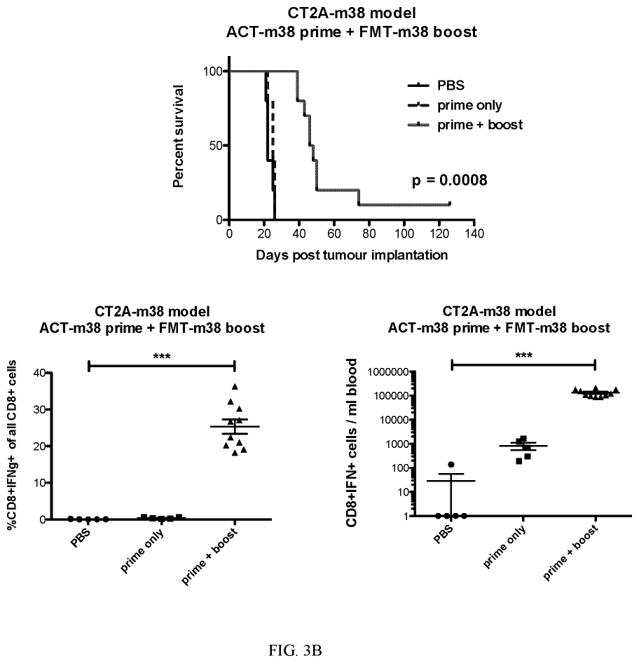

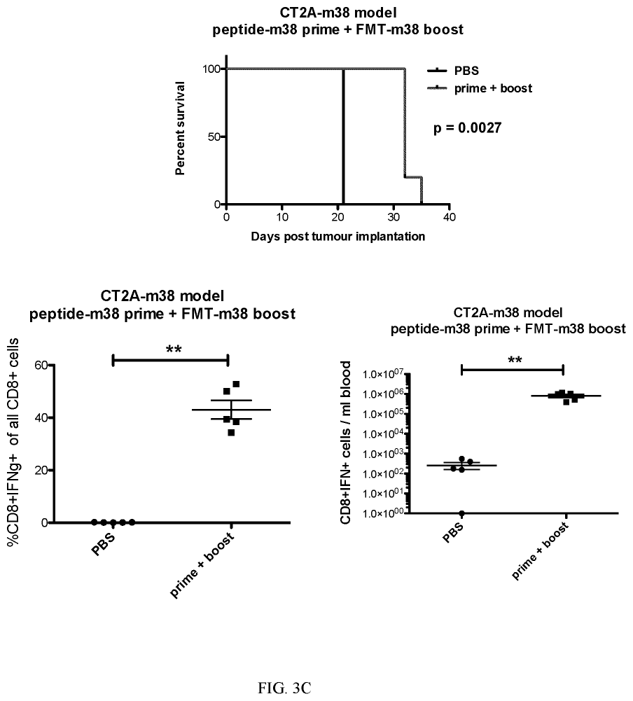

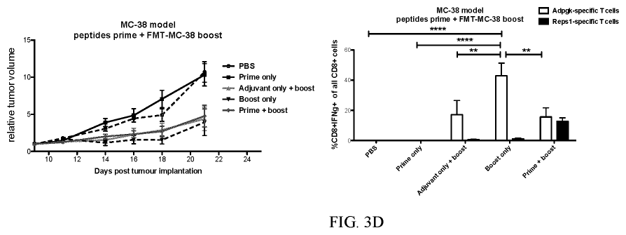

Interestingly, the authors observed that these tumour cells were more aggressive in vivo than the wild type CT2A cells as illustrated by MRI imaging (FIG. 9A). The prime:boost treatment with AdV-m38 and FMT-m38 (administered first intravenously and 2 days later intracranially) significantly increased the frequencies (5.2% vs 2.35% and 0.01%, P<0.0001 for prime:boost, prime only, and PBS respectively (FIG. 3A)) and numbers (4.2×10⁴ cells vs 0.6×10⁴ cells and 0.04×10⁴ cells, P<0.0001 for prime:boost, prime only, and PBS respectively) of m38-specific CD8 T cells, and extended survival (40 days vs 25 and 24 days, P<0.0001, 6/30 (20%) mice were cured in the treatment group) of mice orthotopically implanted with CT2A-m38 cells compared to prime only and PBS controls.

In the next experiment the authors replaced AdV-m38 with ACT-m38 and reduced the number of CT2A-m38 cells from 1×10⁴ to 3×10³ cells. Despite greater immunostimulatory efficiency (frequency of m38-specific T cells: 25.3% vs 0.41% and 0.078% for prime only and PBS control, respectively, P=0.0003, number of m38-specific T cells: 1.3×10⁵ cells vs 820 and 28 cells for prime only and PBS control, respectively, P=0.0003 (FIG. 3B)), similar anti-tumour efficacy was achieved (median survival: 47 days vs 25 and 22 days for prime only and PBS control, respectively, P=0.0008, 1/10 (10%) mice was cured in the treatment group (FIG. 3B)).

Additionally, the authors tested the efficacy of the combination of m38 peptide prime with FMT-m38 (administered only intravenously) in mice implanted with 3×10³ CT2A-m38 cells. This treatment regimen resulted in high increase in frequencies (43.0% vs 0.09%, P=0.0079) and numbers (8.1×10⁵ vs 258 cells, P=0.0079) of m38-specific CD8 T cells and modest survival benefit (32 vs 21 days, P=0.0027) compared to PBS control (FIG. 3C). This suggests that direct injection of FMT virus into the tumour may contribute to anti-tumour efficacy by a mechanism different than inducing high numbers of tumour-specific cytotoxic T cells, however, the impact of chosen prime method on survival cannot be excluded.

Furthermore, the authors of the present disclosure investigated the efficiency of FMT-MC-38 virus in MC-38 subcutaneous mouse tumour model. Tumour-bearing mice were primed with Adpgk and Reps1 long mutant peptides with adjuvant, with adjuvant only or with PBS and boosted with FMT-MC-38 or PBS. Treatment with FMT-MC-38 virus only (with PBS instead of prime) resulted in the highest expansion of Adpgk-specific CD8 T cells (42.9% vs 17.1%, 15.6%, 0.11% and 0.13% in adjuvant+boost, prime+boost, prime only and PBS groups, respectively, P<0.01), and delayed tumour progression (FIG. 3D). FMT-MC-38 was able to boost Adpgk-specific response without prime. On the other hand, a boost of Reps1-specific T cells was only observed when Reps1 peptide prime was used, yet it had no impact on tumour progression and animals' survival (FIG. 3D), suggesting that Reps1 may not be the tumour-rejection antigen.

To summarize, the authors demonstrated in two different in vivo models that a FMT virus-based boost according to the present disclosure generates an immune response against a tumour specific antigen in tumour-bearing mice and extends their survival.

### Experiment 5. TSA-Specific CD8 T Cells Greatly Enhance Efficacy of a FMT Virus-Based Anti-Tumour Treatment

The authors of the present disclosure hypothesized that expansion of tumour specific antigen (TSA)-specific effector T cells contributed greatly to the anti-tumour efficacy of a prime:boost therapy according to the present disclosure. To test this hypothesis, the authors designed an experiment where CT2A-m38 tumour-bearing mice (i) received a prime:boost treatment against m38, or against chicken ovalbumin (OVA)—an irrelevant antigen—or (ii) were adoptively transferred with m38-specific memory T cells, but boosted with FMT virus expressing GFP (FMT-GFP) instead of m38.

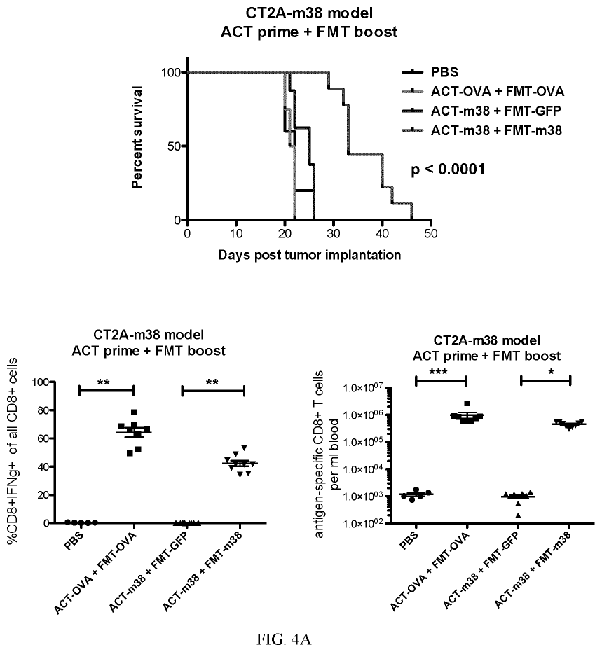

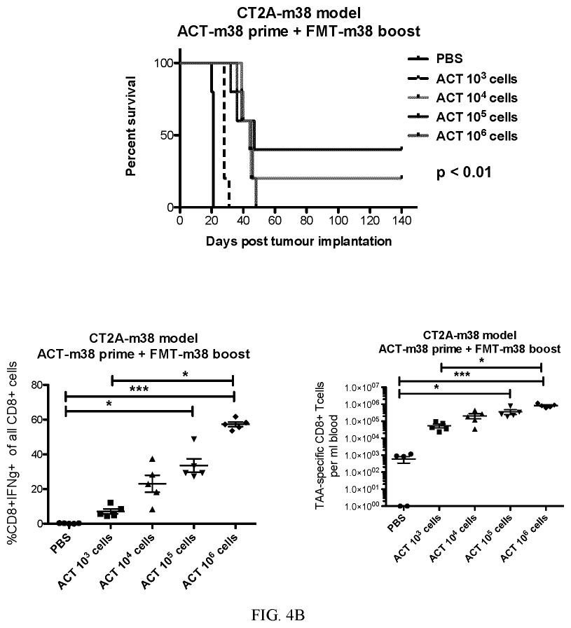

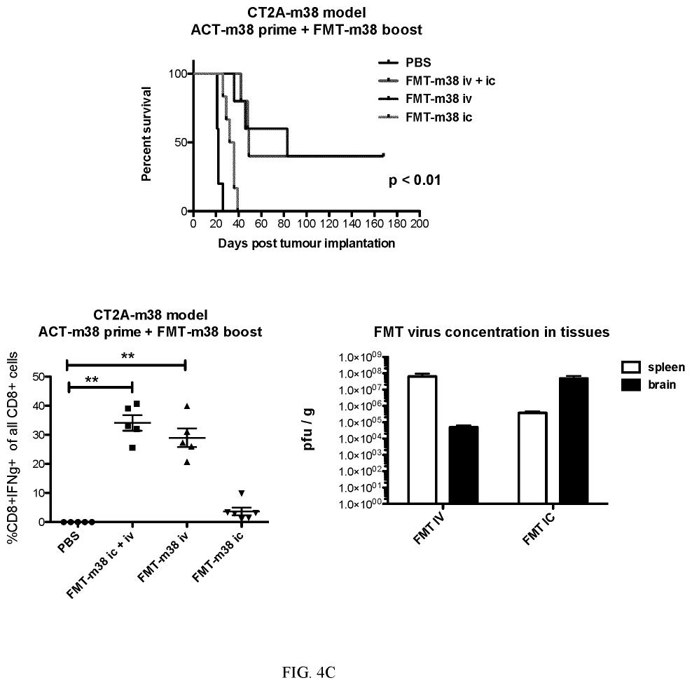

As in previous experiments, a prime:boost treatment using m38 as the shared antigenic peptide induced high frequencies and numbers of m38-specific CD8 T cells and significantly extended animals' survival (FIG. 4A). In contrast, a prime:boost treatment using OVA as the shared antigenic peptide did not provide any survival benefit despite expanding OVA-specific CD8 T cells to high amounts (FIG. 4A), confirming that TSA-specific T cells, but not other T cells, can mediate anti-tumour efficacy. Mice adoptively transferred with m38-specific memory T cells did not benefit from FMT-GFP treatment, as virus without relevant antigen was not able to trigger T cells' differentiation from memory into effector cells (FIG. 4A). These results show that tumour cells killing by TSA-specific effector T cells is a major mechanism contributing to the efficacy of a prime:boost therapy according to the present disclosure.

### Experiment 6. Increasing the Numbers of TSA-Specific CD8 T Cells Improves Therapeutic Efficacy

The authors of the present disclosure aimed to determine whether the T cell-dependency of a prime:boost therapy according to the present disclosure is dose-dependent. For this purpose, the authors primed CT2A-m38 tumour-bearing mice with different doses of ACT-m38 ranging from 10³ to 10⁶ cells and boosted with FMT-m38 virus. All treatments expanded the frequencies and numbers of m38-specific CD8 T cells in a dose-dependent manner (FIG. 4B). ACT-m38 at the lowest dose of 10³ cells resulted in minimal survival benefit compared to PBS control (28 vs 21 days, P=0.0035; FIG. 4B). Increasing the amount of m38-specific CD8 T cells with higher prime doses further extended the animals' survival compared to PBS control and lowest prime dose group (median survival: 44 days, ⅕ (20%) mouse cured, 47 days, ⅖ (40%) mice cured and 45 days at 10⁴, 10⁵ and 10⁶ cells dose groups, respectively, P=0.0035 and P=0.0016 when compared to PBS and 10³ cells dose group, respectively; FIG. 4B). Thus, the numbers of antigen-specific effector T cells directly correlated with anti-tumour efficacy. However, these data also suggest that a saturating treatment dose may have been reached in mice, as no more cures were observed at the prime dose of 10⁶ cells.

### Experiment 7. Anti-Tumour Efficacy Against Glioma can be Achieved with Intravenous FMT Virus Administration

Additionally, the authors of the present disclosure investigated different routes of administration of FMT virus and their effects on anti-tumour efficacy. The authors hypothesized that the intravenous injection would be superior for expanding TSA-specific effector T cells in peripheral blood, especially over the intracranial injection as brain is considered an immune-privileged organ. However, virus injected into the tumour could contribute directly to tumour eradication by oncolytic virus-mediated tumour cell lysis or indirectly by inducing local inflammation, modifying tumour microenvironment and increasing recruitment of cytotoxic T cells into the tumour.

The authors first examined the distribution of FMT virus in the brain and spleen in naïve mice injected intravenously (iv) or intracranially (ic). As expected, more virus was found in the brain following ic injection (mean 1.4×10⁷ pfu that is 40% more than injected dose) compared with iv group (mean 1×10⁴ pfu that is 0.003% of the injected dose) and spleens of iv injected mice contained more virus (mean 1.5×10⁷ pfu that is 5% of the injected dose) than mice receiving virus by ic route (mean 4.95×10⁴ pfu that is 0.5% of the injected dose) (FIG. 4C).

Next, the authors studied the impact of different routes of FMT-m38 administration: 1) ic, 2) iv and 3) iv followed by ic (iv+ic) on the survival of CT2A-m38 tumour-bearing mice primed with ACT-m38. Each treatment induced expansion of m38-specific CD8 T cells (frequencies 3.7%, 30.0% and 34.1% in ic, iv and iv+ic groups, respectively, vs 0.02% in PBS control, P>0.05, P<0.01 and P<0.01, respectively (FIG. 4C)) and extended animals' survival (median survival 34, 83 and 49 days in ic, iv and iv+ic groups, respectively, vs 22 days in PBS control, P=0.0021, P=0.0019 and P=0.0019, respectively (FIG. 4C)). Noteworthy, iv and iv+ic boosting regimens were superior to ic injection (P=0.0073 and P=0.0015, respectively) and resulted in 20% cure rate (⅖ mice). No significant difference was observed between iv and iv+ic groups. Summarizing, an FMT-based boost according to the present disclosure administered intravenously induces antigen-specific response of higher magnitude and results in prolonged survival compared to intracranial injection, mainly due to higher amounts of infectious viral particles migrating to the spleen resulting in enhanced TSA presentation to memory T cells. However, these data do not rule out the possible benefit of injecting FMT-m38 virus directly into the tumour in addition to intravenous prime:boost treatment.

### Experiment 8. Pre-Existing Immunity Against a TSA Extends Survival of Mice Challenged with Tumour, but is not Sufficient for Complete Tumour Rejection

In order to assess whether a pre-existing pool of TSA-specific CD8 effector T cells would prevent the tumour progression following tumour cell implantation, the authors of the present disclosure injected CT2A-m38 intracranially in the mice previously treated with the prime:boost therapy in the experiment, discussed above, entitled “Immune response induced by an FMT virus boost can be sustained over prolonged periods of time” at 281/161 days post 1st/2nd boost (presented in FIG. 2A-2E).

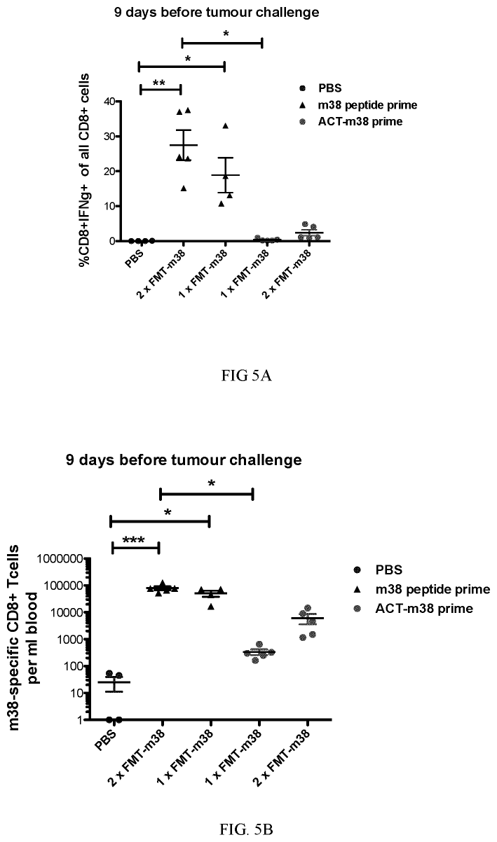

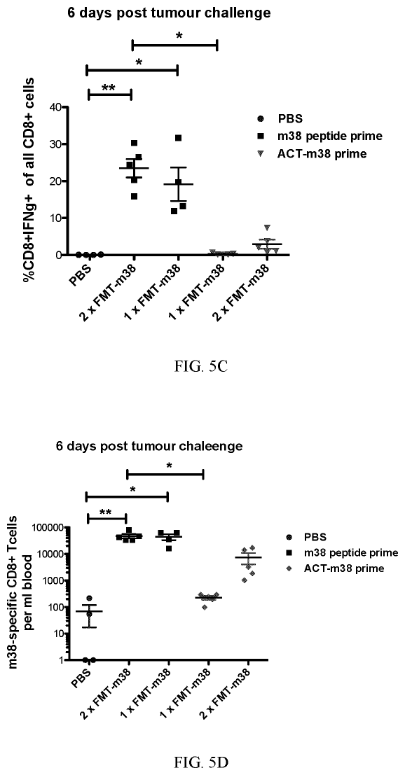

The amount of m38-specific CD8 T cells was similar before and after tumour challenge, however, varied between groups with different treatment regime (FIG. 5A-5D). All prime:boost treated mice survived significantly longer than PBS control group (median survival: 32, 34.5, 35, 35 days for mice receiving m38 peptide prime with two FMT-m38 boosts, m38 peptide prime with one FMT-m38 boost, ACT-m38 prime with two FMT-m38 boosts, ACT-m38 prime with one FMT-m38 boost, respectively, vs 21 days for PBS control group, P<0.05 (FIG. 5E)). However, all mice eventually succumbed to tumour regardless of the amount of pre-existing m38-specific CD8 T cells and the median survival of prime:boost treated mice was very similar to the outcomes of mice treated with FMT-m38 in most of the therapeutic experiments the authors have conducted. These results suggest either an inefficient recruitment of effector T cells to the tumour, their reduced functionality (exhaustion), or inefficiency without adjuvant therapy.

### Experiment 9. Intracranial Injection of FMT-m38 Virus Promotes Anti-Tumour Immune Response within the Brain Tumour Microenvironment

To examine the impact of an exemplary boost according to the present disclosure on the tumour microenvironment, the authors harvested the tumour tissue from mice bearing CT2A-m38 tumours primed with m38 peptide and boosted with FMT-m38 virus intracranially or intravenously.

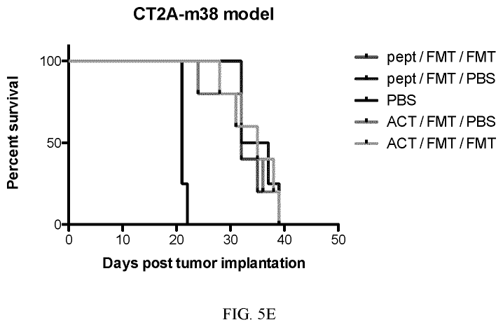

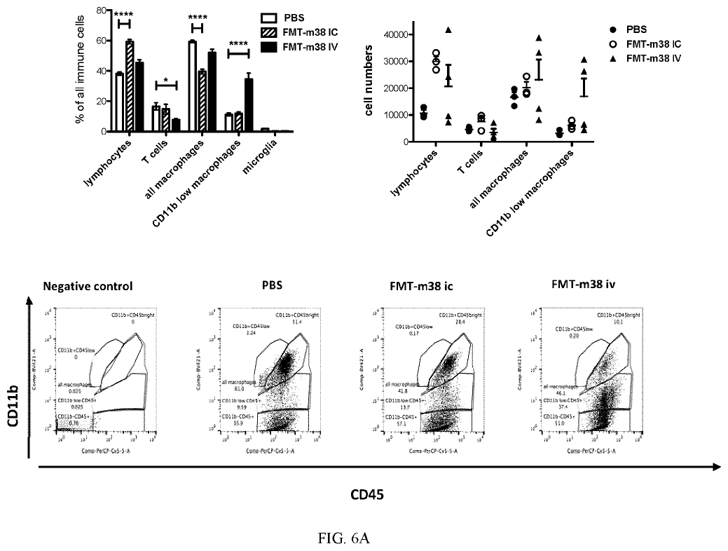

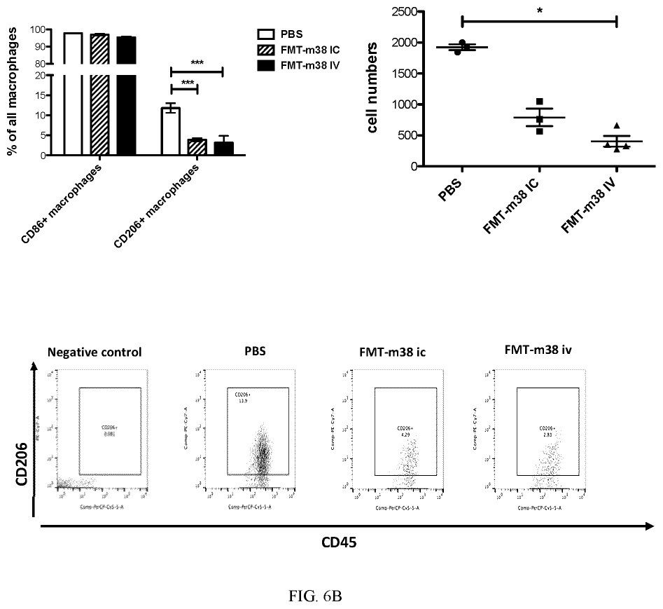

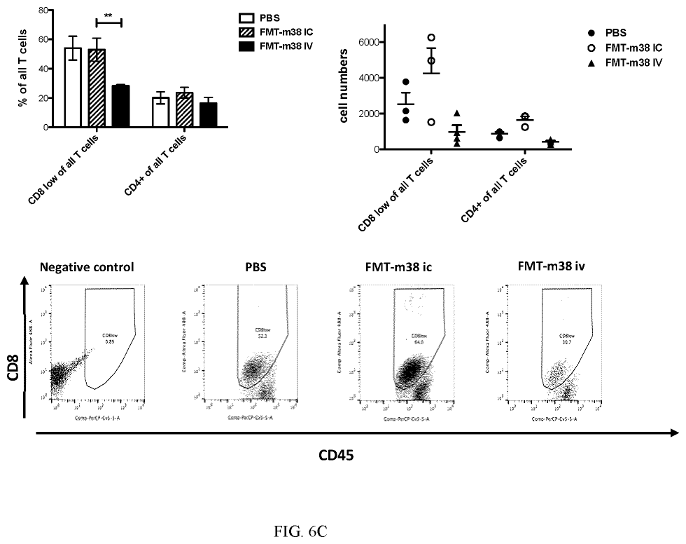

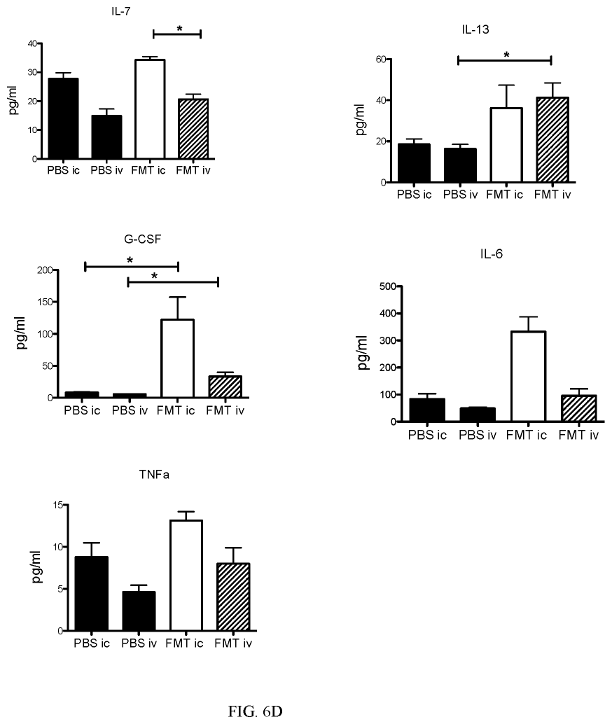

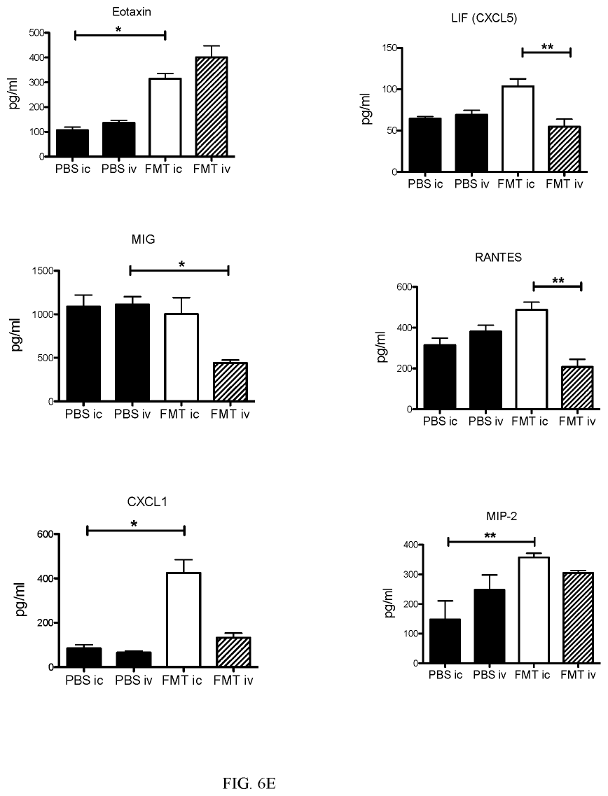

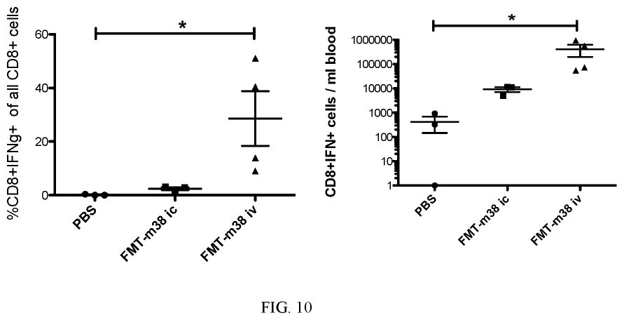

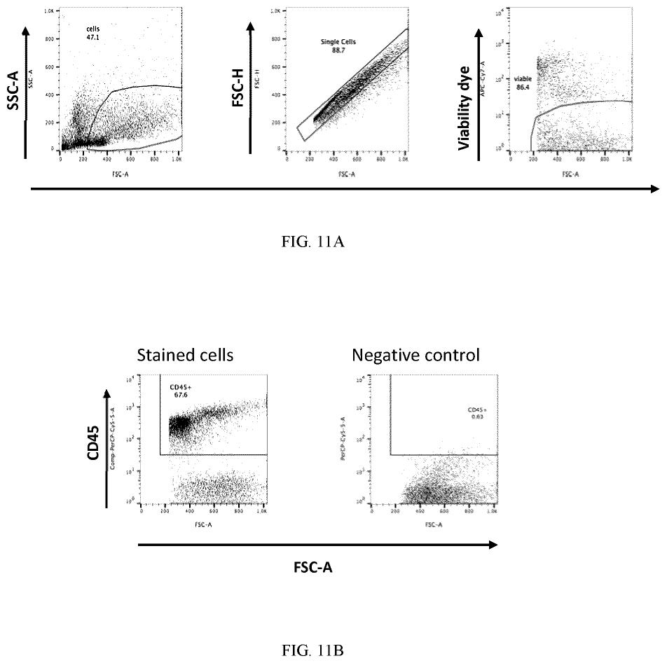

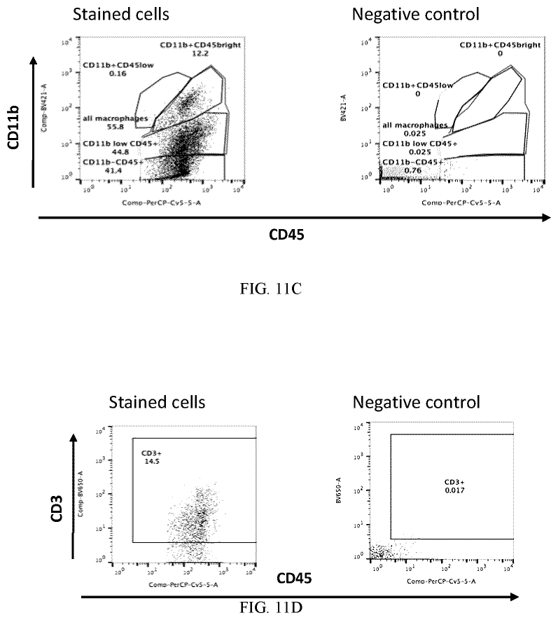

Blood sample was collected 6 days after boost, just before the tumour tissue harvest, in order to confirm the expansion of peripheral m38-specific CD8 T cells (FIG. 10). Compared to control PBS group, the ic injection of FMT-m38 virus increased the recruitment of lymphocytes, including T cells, into the tumour, while the amounts of macrophages and microglia remained unchanged (FIG. 6A). Unexpectedly, the authors detected decreased T cell infiltration in the iv injection group (FIG. 6A). Interestingly, the authors observed reduced expression of CD11 b in the macrophage population (illustrated as CD11b^{low} macrophage population in FIG. 6A) in the iv injection group compared to both ic injection group and PBS control. Both treatment regimens diminished the numbers of macrophages expressing CD206—one of the markers of M2-polarization, while the expression level of CD86 co-stimulatory molecule remained the same as in the control group (FIG. 6B). Among tumour-infiltrating lymphocytes (TILs), the authors observed increased amounts of both CD4 and CD8 T cells (defined as CD8_{low} in FIG. 6C) in the ic injection group compared to control and iv injection groups (FIG. 6C). In each group, including control, over 90% of CD8 T cells expressed CD137—a marker of activation induced by TCR stimulation.

Additionally, in a separate experiment, the authors compared the cytokine and chemokine profiles of tumour microenvironment following wild-type FMT virus ic or iv injection. Tumours harvested from mice injected with FMT virus by ic route had increased concentration of IL-7 cytokine (P<0.05) important for maintenance of memory T cell pools and pro-inflammatory cytokines IL-6 and TNFα (not statistically significant) compared to tumours from iv injected mice (FIG. 6D). On the other hand, the authors also observed higher level of IL-13 cytokine that inhibits Th1-type T cell responses in both ic and iv (P<0.05) injection groups compared to PBS controls (FIG. 6D). Compared to PBS controls, both injection groups also manifested with elevated expression of granulocyte-colony stimulating factor (G-CSF) supporting the proliferation and differentiation of neutrophils (FIG. 6D). Moreover, ic injection of FMT virus induces granulocyte-attracting chemokine environment (FIG. 6E) as illustrated by increased concentration of Eotaxin (P<0.05 compared to PBS control), CXCL5 (P<0.01 compared to iv group), CXCL1 (P<0.05 compared to PBS control) and MIP-2 (P<0.01 compared to PBS control). Interestingly, iv virus injection resulted in decreased level of MIG—a molecule attracting Th1 cells and of RANTES—a chemokine recruiting whole spectrum of immune cells: NK cells, T cells, DCs, basophils, eosinophils and monocytes (FIG. 6E).

Taken together, these results emphasize that injecting an FMT-based boost directly into the tumour in addition to intravenous immunization induces changes within the tumour microenvironment favourable for anti-tumour immune response as demonstrated by increased infiltration of activated CD8 T cells, reduced numbers of CD206+ macrophages and pro-inflammatory cytokine secretion.

Animal Studies

All C57Bl/6 and C57Bl/6-Ly5.1 mice were purchased from Charles River Laboratories.

Generating Cellular Product for Adoptive Cell Transfer (ACT)

Male transgenic C57BL/6N-Tg(Tcra, Tcrb)329Biat (Maxi-m38) mice—kindly provided by Dr Annette Oxenius (ETH Zurich, Switzerland) were paired with C57Bl/6-Ly5.1 female mice to establish a colony. Female OT-1 mice were purchased from Jackson Laboratories.

To generate cellular product for adoptive cell transfer (ACT), spleens from female Maxi-m38 or OT-1 mice were extracted and spleenocytes were isolated and cultured in RPMI medium supplemented with 10% FBS, non-essential amino acids, 55 mM 2β-mercaptoethanol, HEPES buffer (Stem Cell), Penicillin-Streptomycin and central memory T cell (Tcm) enrichment cocktail kindly provided by Dr Yonghong Wan (McMaster University, Hamilton, Canada) for 6-7 days.

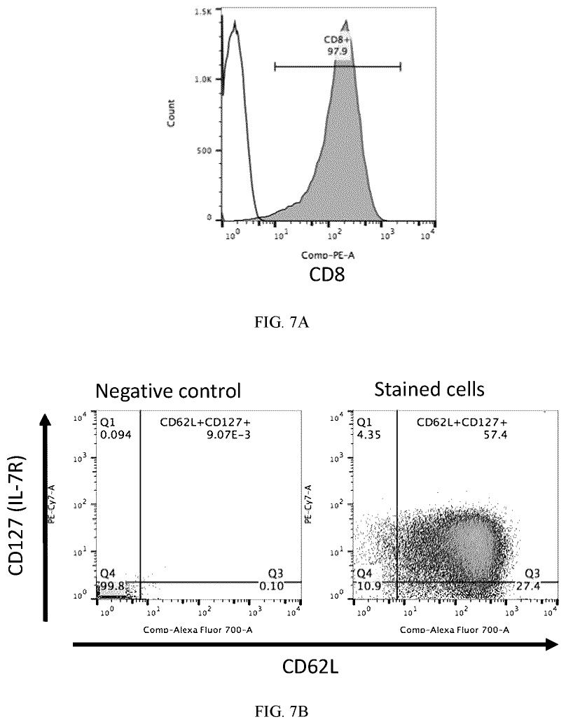

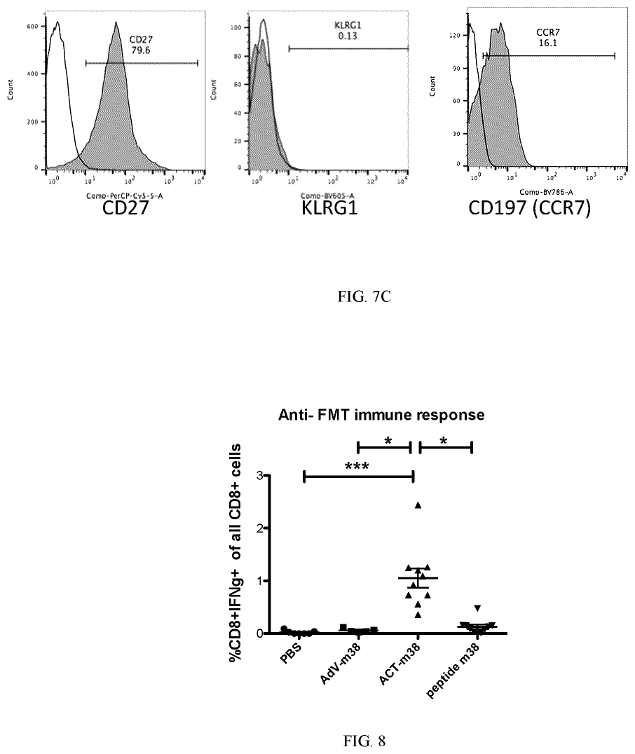

Peptides: m38 or chicken ovalbumin (OVA) immunodominant epitope were added only at the start of culture at 1 μg/ml. The cells were passaged once or twice depending on the density. For ACT cells were harvested by pipetting, washed 2× with DPBS counted using hematocytometer with Trypan blue staining and re-suspended in DPBS. Part of the cellular product was put aside for phenotyping by flow cytometry the same day or the day after ACT. The memory phenotype was confirmed by staining with fluorochrome-conjugated antibodies: CD8-PE, CD127-PE-Cy7, CD27-PerCP-Cy5.5, KLRG1-BrilliantViolet605, CD62L-AlexaFluor700 and CCR7(CD197)-BrilliantViolet786. Fixable eFluor450 viability dye (eBioscience) was used to exclude dead cells. Over 95% of cells were CD8+ T cells and the frequency of Tcm cells defined as CD127+CD62L+ cells ranged from 40 to 60% (FIG. 7).

Vaccination Studies in Naïve Mice

7-10 weeks old female C57Bl/6 mice were primed at day 0 with:

1) 1×10⁸ plaque forming units (pfu) of adenovirus (AdV) expressing DCT (AdV-DCT) or m38 (AdV-m38) by bilateral intramuscular injection,

2) adoptive cell transfer (ACT) of m38- or OVA-specific CD8 memory T cells (ACT-m38 or ACT-OVA) at the dose of 1×10⁵ cells intravenously (iv) or 3) intraperitoneally (ip) with 50 μg of one or more peptides (Biomer Technology,) with adjuvant: 30-50 μg of anti CD40 antibody (BioXCell) and 10-100 μg of poly I:C.

Mice were boosted intravenously 9-14 days later with 3×10⁸ pfu FMT virus expressing m38 (FMT-m38), DCT (FMT-DCT), GFP (FMT-GFP) or MC-38-derived neo-epitopes Adpgk, Dpagk1 and Reps1 (FMT-MC-38). The blood was collected 5-7 days after boost and in some cases at later time points for quantification of antigen-specific T cells by ex vivo peptide stimulation and intracellular cytokine staining (ICS) assay. In one experiment mice were given 3×10⁸ pfu FMT-m38 virus for the 2nd time 120 days following the 1st boost. In another one, mice received 3×10⁸ pfu FMT-MC-38 virus for the 2nd time 35 days after 1st boost and for the 3rd time 124 days post 2nd boost.

Efficacy Experiments in Brain Tumour-Bearing Mice

For brain tumour efficacy studies, 7-10 weeks old female C57Bl/6 mice were injected intracranially (ic) at day 0 with CT2A-m38 cells and re-suspended in serum-free DMEM medium at a position 2.5 mm to the right and 0.5 mm anterior to bregma, 3.5 mm deep, using Hamilton syringe and infusion pump attached to stereotaxic frame. In the experiments presented in FIG. 3A and discussed with regard to Experiment 4, above, the authors of the present disclosure injected 1×10⁴ cells, in all other experiments, they injected 3×10³ cells. Mice were primed at day 3 with 1×10⁹ pfu of AdV-m38 or with 50 μg m38 peptide with adjuvant: 30 μg of anti CD40 antibody (BioXCell) and 10 μg of poly I:C. Alternatively, mice were primed at day 11 with ACT-OVA at 1×10⁶ cells or ACT-m38 at doses: 1×10⁶ cells in the experiment presented in FIG. 4A (Experiment 5, discussed above), or 1×10⁵ cells in other experiments except the dose response study (FIG. 4B; Experiment 6). FMT-m38, FMT-OVA or FMT-GFP were administered either ic at day 12 at a dose of 1×10⁷ pfu at the same position but 2.5 mm deep or iv at day 14 at a dose of 3×10⁸ pfu, or both.

Blood was collected 5 days after ic boost or 7 days after iv boost (day 19 post tumour implantation) for quantification of antigen-specific CD8 T cells. Mice were monitored daily for the onset of symptoms like piloerection, facial grimace, hunched back, respiratory distress or neurological symptoms (head tilt, circling, seizure) and euthanized when reached endpoint. Visible head tumours frequently occurred, however, there was always also intracranial tumour as well evident upon dissection post mortem. Whenever the cause of endpoint was in doubt, mice were dissected post mortem to confirm the presence of intracranial tumour. No virus-related acute toxicities were observed after either iv or ic FMT-m38 injection. Mice would frequently lose weight after immunization with FMT virus, however, never more than 15% and they would regain the baseline body mass within a week.

Efficacy Experiments in MC-38 Tumour-Bearing Mice

8 weeks old female C571316 mice were injected subcutaneously at day 0 with 1×10⁵ MC-38 cells re-suspended in serum-free DMEM medium. Next day (day 1) mice were primed with 50 μg of Adpgk and Reps1 long mutant peptides with adjuvant: 30 μg of anti CD40 antibody (BioXCell) and 10 μg of poly I:C, with adjuvant alone or with PBS. On day 9 tumour were measured and only mice with tumour size 80-130 mm³ were included in the study. On day 10 mice were injected with 3×10⁸ pfu FMT-MC-38 virus (one peptide-primed group, adjuvant-primed group and one PBS-primed group) or PBS (one peptide primed group and one PBS primed group). Tumours were measured next day and twice a week until mice reached endpoint: tumour size above 1000 mm³ or bleeding ulcers. Tumour volume was calculated with formula: (length×width×depth)/2. No virus-related acute toxicities were observed following FMT-MC-38 injection.

PBMC Isolation, Stimulation, and Intracellular Cytokine Staining (ICS) Assay

Blood was collected from mice into heparinized blood collection tubes by puncturing the saphenous vein. The blood volume was measured and blood was transferred into 15 ml conical tubes for erythrocyte lysis with ACK lysis buffer. The PBMCs were re-suspended in RPMI medium supplemented with 10% FBS, non-essential amino acids, 55 mM 2β-mercaptoethanol, HEPES buffer (Stem Cell) and Penicillin-Streptomycin and transferred to 96 well round-bottom plates. Each sample was split into either 3 wells (antigen stimulation, FMT-derived epitope stimulation and no-stimulation control) or 4 wells in experiments with MC-38 derived epitopes (1 for each epitope separately and unstimulated control). For unstimulated control, 0.1-0.4% DMSO (Sigma) in RPMI was added as the peptides stock solutions were made in DMSO. Blood samples from naïve mice were used for extra controls of peptide stimulation, for staining-negative controls and for PMA and lonomycin stimulated (at 100 ng/ml and 1 μg/ml, respectively) positive controls. The peptides were added at a concentrations 0.5 μg/ml, 1 μg/ml, 1 μg/ml or 5 μg/ml for OVA, m38, FMT or MC-38 peptides, respectively. Following 1 h incubation at 37° C., 5% CO₂, GolgiPlug (BD Biosciences) was added to each well at 0.2 μl per well and incubated for 4 h more. Cells were then washed, transferred to 96 well v-bottom plates (EverGreen) and stored overnight at 4° C. Next day ICS assay was performed. Briefly, cells were washed with FACS buffer (0.5% BSA in PBS), stained with CD8-PE, TCR-BrilliantViolet711 and CD45.1-PerCP-Cy5.5 antibodies and Fixable eFluor450 viability dye (eBioscience), washed with FACS buffer, fixed and permeabilized with Fixation and permeabilization kit (BD Bioscienses), stained with IFNγ-AlexaFluor647, TNFα-PE-Cy7 and IL-2-BrilliantViolet605 antibodies and re-suspended in FACS buffer. Data were acquired on BD LSR Fortessa X20 flow cytometer with HTS unit (BD Biosciences) and data were analysed using FlowJo (TriStar) software. The debris and doublets were excluded by gating on FSC vs SSC and FSC-A vs FSC-H, respectively. Viable cells were gated based on viability dye stain. Next, CD8-positive and TCR-positive cells were gated and within this population the expression of IFNγ, TNFα and IL-2 was examined. Cell numbers were calculated with the following formula:

$$N [ cell number \text{/} ml ] = \frac{Ns - Nu}{( \frac{Vm}{W} ) * Vf} * 1000$$

where N—resulting positive cell number per 1 ml of blood, Ns—number of positive cells in the well containing peptide, Nu—number of positive cells in unstimulated control, Vm—total blood volume collected from animal, W—number of wells the blood sample was distributed into, Vf—fraction of sample volume used for data acquisition by flow cytometry i.e. 80 μl out of 130 μl.

Characterization of Tumour Microenvironment

Phenotyping of Tumour-Infiltrating Immune Cells

7 weeks old female C57Bl/6 mice were injected intracranially (ic) at day 0 with 3×10³ CT2A-m38 cells and re-suspended in serum-free DMEM medium at a position 2.5 mm to the right and 0.5 mm anterior to bregma, 3.5 mm deep, using Hamilton syringe and infusion pump attached to stereotaxic frame. At day 3, mice were primed with 50 μg m38 peptide with adjuvant: 30 μg of anti CD40 antibody (BioXCell) and 10 μg of poly I:C or with PBS. 9 days later mice were boosted with either 1×10⁷ pfu FMT-m38 injected ic at the same position but 2.5 mm deep, with 3×10⁸ pfu FMT-m38 iv, or with PBS ic. 6 days after boost blood was collected to confirm the presence of m38-specific CD8 T cells in peripheral blood and afterwards mice were euthanized and tumour tissue was collected. The tumour tissue was dissociated with Neural Tissue Dissociation kit (Miltenyi Biotech) and the cells purified with Percoll gradient method. Cells were then kept overnight at 4° C. The next day, cells were washed with FACS buffer and stained with fluorochrome-conjugated antibodies: CD11b-BrilliantViolet421, CD4-BrilliantViolet510, CD86-BrilliantViolet605, CD3-BrilliantViolet650, F4/80-BrilliantViolet711, CD137-BrilliantViolet785, CD8-AlexaFluor488, CD45-PerCP-Cy5.5, NKp46-PE, CD206-PE-Cy7 and with m38-tetramer-APC. Fixable near-IR viability dye (eBioscience) was used to exclude dead cells. Data were acquired using BS LSR Fortessa X20 flow cytometer (BD Biosciences) and analysed with FlowJo (TriStar) software.

Statistics

Kaplan-Meier survival curves were generated in GraphPad version 5.0f (Prism) software and compared using Log-rank (Mantel-Cox) test. P value below 0.05 was considered significant. Frequencies and numbers of immune cells, cytokine and chemokine concentrations were compared across treatment groups in GraphPad version 5.0f (Prism) software using statistical test indicated in the figure legend. P value below 0.05 was considered significant.

In the preceding description, for purposes of explanation, numerous details are set forth in order to provide a thorough understanding of the examples. However, it will be apparent to one skilled in the art that these specific details are not required. Accordingly, what has been described is merely illustrative of the application of the described examples and numerous modifications and variations are possible in light of the above teachings.

Since the above description provides examples, it will be appreciated that modifications and variations can be effected to the particular examples by those of skill in the art. Accordingly, the scope of the claims should not be limited by the particular examples set forth herein, but should be construed in a manner consistent with the specification as a whole.

1. A Farmington virus comprising a nucleic acid that is capable of expressing a tumour associated antigen or an epitope thereof.

2. The Farmington virus of claim 1, wherein the genomic backbone of the Farmington virus encodes a protein having at least 90% sequence identity with any one of SEQ ID NOs 3-7.

3. The Farmington virus of claim 2, wherein the genomic backbone of the Farmington virus encodes a protein having at least 95% sequence identity with any one of SEQ ID NOs 3-7.

4. The Farmington virus of any one of claims 1-3, wherein the tumour associated antigen is a foreign antigen.

5. The Farmington virus of claim 4, wherein the foreign antigen comprises E6 protein from HPV or E7 protein from HPV.

6. The Farmington virus of claim any one of claims 1-3, wherein the tumour associated antigen is a self antigen.

7. The Farmington virus of claim 6, wherein the self antigen is MAGEA3.

8. The Farmington virus of claim any one of claims 1-3, wherein the tumour associated antigen is a neoepitope.

9. The Farmington virus of any one of claims 1-7, wherein the Farmington virus induces an immune response against the tumour associated antigen in a mammal to whom the Farmington virus is administered.

10. The Farmington virus of claim 9, wherein the mammal has been previously administered a prime that is immunologically distinct from the Farmington virus.

11. The Farmington virus of claim 10, wherein the prime is
(a) a virus comprising a nucleic acid that is capable of expressing the tumour associated antigen or an epitope thereof;
(b) T-cells specific for the tumour associated antigen; or
(c) a peptide of the tumour associated antigen.

12. The Farmington virus of any one of claims 1-11, further encoding a cell death protein.

13. A composition comprising a Farmington virus comprising a nucleic acid that is capable of expressing a tumour associated antigen or an epitope thereof, the composition being formulated to induce an immune response in a mammal against the tumour associated antigen.

14. A composition comprising a Farmington virus and an antigenic protein that includes an epitope from a tumour associated antigen, wherein the Farmington virus is separate from the antigenic protein, the composition being formulated to induce an immune response in a mammal against the tumour associated antigen.

15. A heterologous combination prime:boost therapy for use in inducing an immune response in a mammal, wherein the prime is formulated to generate an immunity in the mammal to a tumour associated antigen, and the boost comprises: a Farmington virus comprising a nucleic acid that is capable of expressing a tumour associated antigen or an epitope thereof and is formulated to induce the immune response in the mammal against the tumour associated antigen.

16. A method of enhancing an immune response in a mammal having a cancer, the method comprising a step of:
administering to the mammal a composition comprising a Farmington virus comprising a nucleic acid that is capable of expressing a tumour associated antigen or an epitope thereof,
wherein the mammal has been administered a prime that is directed to the tumour associated antigen or an epitope thereof; and
wherein the prime is immunologically distinct from the Farmington virus.

17. The method of claim 16, wherein the mammal has a tumour that expresses the tumour associated antigen.

18. The method of claim 16 or 17, wherein the cancer is brain cancer.

19. The method of claim 18, wherein the brain cancer is glioblastoma.

20. The method of claim 16 or 17, wherein the cancer is colon cancer.

21. The method of any one of claims 16-20, wherein the Farmington virus is capable of expressing an epitope of the tumour associated antigen.

22. The method of any one of claims 16-20, wherein the prime is directed to an epitope of the tumour associated antigen.

23. The method of claim 22, wherein the prime is directed the same epitope of the tumour associated antigen as the epitope encoded by the Farmington virus.

24. The method of any one of claims 16-23, wherein the prime comprises:
(a) a virus comprising a nucleic acid that is capable of expressing the tumour associated antigen or an epitope thereof;
(b) T-cells specific for the tumour associated antigen; or
(c) a peptide of the tumour associated antigen.

25. The method of claim 24, wherein the prime comprises a virus comprising a nucleic acid that is capable of expressing the tumour associated antigen or an epitope thereof.

26. The method of claim 25, wherein the prime comprises a single-stranded RNA virus.

27. The method of claim 26, wherein the single-stranded RNA virus is a positive-strand RNA virus.

28. The method of claim 27, wherein the positive-strand RNA virus is a lentivirus.

29. The method of claim 26, wherein the single-stranded RNA virus is a negative-strand RNA virus.

30. The method of claim 25, wherein the prime comprises a double-stranded DNA virus.

31. The method of claim 30, wherein the double-stranded DNA virus is an adenovirus.

32. The method of claim 31, wherein the adenovirus is an Ad5 virus.

33. The method of claim 24, wherein the prime comprises T-cells specific for the tumour associated antigen.

34. The method of claim 24, wherein the prime comprises a peptide of the tumour associated antigen.

35. The method of claim 28, wherein the prime further comprises an adjuvant.

36. The method of claim any one of claims 16-35, wherein the mammal is administered the composition at least 9 days after the mammal was administered the prime.

37. The method of any one of claims 16-36, wherein the mammal is administered the composition no more than 14 days after the mammal was administered the prime.

38. The method of any one of claims 16-37, further comprising a second step of administering to the mammal a composition comprising a Farmington virus comprising a nucleic acid that is capable of expressing a tumour associated antigen or an epitope thereof.

39. The method of claim 38, wherein the second step of administering is performed at least 50, at least 75, at least 100, or at least 120 days after the first step of administering.

40. The method of claim 38 or 39, further comprising a third step of administering to the mammal a composition comprising a Farmington virus comprising a nucleic acid that is capable of expressing a tumour associated antigen or an epitope thereof.

41. The method of claim 40, wherein the third step of administering is performed at least 50, at least 75, at least 100, or at least 120 days after the second step of administering.

42. The method of any one of claims 16-41, wherein at least one step of administering is performed by a systemic route of administration.

43. The method of any one of claims 16-41, wherein at least one step of administering is performed by a non-systemic route of administration.

44. The method of any one of claims 16-41, wherein at least one step of administering is performed by injection directly into a tumour of the mammal.

45. The method of any one of claims 16-41, wherein at least one step of administering is performed intracranially.

46. The method of any one of claims 16-41, wherein at least one step of administering is performed intravenously.

47. The method of any one of claims 16-41, wherein at least one step of administering is performed both intravenously and intracranially.

48. The method of any one of claims 16-47, wherein the frequency of T cells specific for the tumour associated antigen is increased after the step of administering.

49. The method of claim 48, wherein the T cells comprise CD8 T cells.

50. The method of any one of claims 16-49, wherein the mammal's survival is extended compared to that of a control mammal who is not administered the composition.

51. The method of claim 50, wherein the control mammal is administered a prime directed to the tumour associated antigen, wherein the prime is immunologically distinct from the composition.

52. The method of any one of claims 16-51, wherein the frequency of T cells specific for the Farmington virus increases by no more than 3% after the step of administering.

53. The method of claim 52, wherein the frequency of CD8 T cells specific for the Farmington virus increases by no more than 3% after the step of administering.


SEQUENCE LISTING (37 sequences)

SEQ ID NO: 1 - DNA, 12423 bp, Farmington rhabdovirus
  promoter (134)..(149)
  misc_feature (206)..(1444): ORF 1
  promoter (1562)..(1578)
  misc_feature (1640)..(2590): ORF 2
  promoter (2799)..(2813)
  misc_feature (2894)..(3340): ORF 3
  promoter (3457)..(3469)
  misc_feature (3603)..(5717): ORF 4
  promoter (5766)..(5780)
  misc_feature (5832)..(12221): ORF 5
ttacgacgcataagctgagaaacataagagactatgttcatagtcaccctgtattcattattgacttttatgacctattattcgtgaggtcatatgtgaggtaatgtcatctgcttatgcgtttgcttataagataaaacgatagacccttcacgggtaaatccttctccttgcagttctcgccaagtacctccaaagtcagacgatggctcgtccgctagctgctgcgcaacatctcataaccgagcgtcattcccttcaggcgactctgtcgcgggcgtccaagaccagagccgaggaattcgtcaaagatttctaccttcaagagcagtattctgtcccgaccatcccgacggacgacattgcccagtctgggcccatgctgcttcaggccatcctgagcgaggaatacacaaaggccactgacatagcccaatccatcctctggaacactcccacacccaacgggctcctcagagagcatctagatgccgatgggggaggctcattcacagcgctgcccgcgtctgcaatcagacccagcgacgaggcgaatgcatgggccgctcgcatctccgactcagggttggggcctgtcttctatgcagccctcgctgcttacatcatcggctggtcaggaagaggagagactagccgcgtgcagcagaacataggtcagaaatggctgatgaacctgaacgcaatcttcggcaccacgatcacccatccaacaaccgtgcgtctgccaatcaacgtcgtcaacaacagcctcgcagtgaggaacggacttgctgccacactctggctatactaccgttcatcacctcagagtcaggacgcgttcttctatgggctcatccgtccctgttgcagtggatatctcggcctgctacatcgggtgcaggagattgatgagatggagccggacttcctcagtgacccccggatcatccaggtgaatgaggtctacagtgcactcagagccctggttcaactgggaaacgacttcaagaccgccgatgatgagcccatgcaggtctgggcgtgcaggggaatcaacaacggatatctgacatatctctcagaaactcctgcgaagaaaggagctgttgtgcttatgtttgcccaatgcatgctgaagggcgactctgaggcctggaacagctaccgcactgcaacctgggtgatgccctattgcgacaatgtggccctaggagcgatggcaggctacatccaagcccgccagaacaccagggcatatgaggtctcagcccagacaggtctcgacgtcaacatggccgcggtcaaggactttgaggccagttcaaaacccaaggctgctccaatctcgctgatcccacgccccgctgatgtcgcatcccgcacctctgagcgcccatctattcctgaggttgacagcgacgaagagctcggaggaatgtaaaccaataagcttcactgccggtagtttaggcatacacacgcagttccgttatccatcacacccgtcccttcttttatgctgctattatttcagttgctaagcttcctgatttgattaacaaaaaaccgtagacctcctacgtgaggtatagctagaaattggttctatcggttgagagtctttgtactattagccatggaggactatttgtctagcttagaggccgcgagagagctcgtccggacggagctggagcccaagcgtaacctcatagccagcttagagtccgacgatcccgatccggtaatagcgccagcggtaaaaccaaaacatcccaagccatgcctgagcactaaagaagaggatcatctcccctctcttcgcctactattcggcgcaaaacgagacacctcggtgggcgtagagcagactctccacaagcgtctctgcgcttgtctcgacggttacctgaccatgacgaagaaagaggccaatgcctttaaggccgcggctgaagcagcagcattagcagtcatggacattaagatggagcatcagcgccaggatctagaggatctgaccgctgctatccctaggatagaattcaaactcaatgccatcctggaaaacaacaaggagatagccaaggctgtaactgctgctaaggagatggagcgggagatgtcgtggggggaaagcgccgccagctcgctcaagtctgtcaccctagatgagtcgtttaggggccctgaagagctttcagagtcatttggcatccgatataaggtcagaacctggaatgagttcaagaaggcgctggaaaccagcattgtggacctgaggcctagccctgtttcatttagggaattacggactatgtggctgtctcttgacacctcctttaggctcattgggtttgccttcattcccacatgcgagcgcctggagaccaaagccaaatgcaaggagacaaggactctactcccccttgcagagtcgatcatgcgaagatgggacctgcgggatccaaccatcttggagaaagcctgcgtagtaatgatgatccgtgggaatgagattgcatcgctgaatcaggtaaaagatgttctcccgaccacaattcgtgggtggaagatcgcttattagtcactgctcccattagtcccactagacggcatacttccattccgccctttaattcccctgtcagacactcatgctccgaaatcactaaccatccttgtccaccaagcaatacgcatattcagtagcactgcatctcgccctccccctatcaagccccagcgctgcagatcttcaccacatatatacatgcatcaactacatgtgatttagaaaaaaccagacccttcacgggtaatagcctaactcacgaacgttcctctcgtttcgtatgataaggccttaagcattgtcgatacggtcgttatgcgtcggttctttttaggagagagcagtgcccctgcgagggactgggagtccgagcgacctcccccctatgctgttgaggtccctcaaagtcacgggataagagtcaccgggtacttccagtgcaacgagcgtccgaaatccaagaagaccctccacagcttcgccgtaaaactctgcgacgcaattaagccggttcgagcggatgctcccagcttgaagatagcaatatggacggctctagatctggccttcgtgaaacctcccaatggaactgtaacaatagatgcggcggtgaaagctacaccgctaatcgggaacacccagtacaccgtaggcgatgaaatcttccagatgctagggagaaggggtggcctgatcgtcatcaggaacttaccccatgattatcctcgaacgttgattgagttcgcctctcccgagccttgagcaccagggcatcggtccgcccgccctgtgatctcccgtagccgggctcagcgatcaagccggcccgggtcgggggggactggtgcaacacaaggggcggcagtggacgctgattaacaaaaaaccacctatatagacccctcacggtcttagactctgttgccagctgacaaccaacacacaagacatctctctgattcagccgacccgatcgattcctccccacccaattcctaccaacgcactcctcacaagctccaccatgctcaggatccagatccctccgattgctatcattctggtaagtctcctcacactcgacctgtccggtgcaaggaggacaaccacacaaagaatccctctccttaatgattcgtgggatttgttctcgagctatggcgacattcccgaagaacttgtcgtataccagaactacagccacaattcctccgagttaccccctcctggcttcgagagatggtacataaaccgaagagtggcagacacttccataccgtgcaggggcccctgtctagtgccctacatccttcatggcctcaatgacacaactgtctctcgacggggaggaggatggcgaaggtccggaatgaagtacccaacccacgctgtcaggctaggcccttcaacagacgacgagagagttgaggaagacatcggctacgtcaatgtctccgcactatcctgcacagggtcgcccgttgagatggcgataccaacaatccccgactgcaccagtgctatccatccacgatccgaggttactgtgcccgtcaagctcgatgtcatgagacgaaatcccaactaccctcccattagagcgtggtcgtgcatcggacagaaaatcaccaaccgatgtgattgggcactcttcggcgagaacctcatatatactcaagttgaagctagctctctagcattcaagcacacaagagcctctcttttgaacgaatccaacgggatagacgctgaaggacgtgcagttccctatatcctcggggatatcgaacccgggtactgccgaaccctattcaacacatgggtctctagtgagatcgtgtcatgcacgcccatcgaacttgtcctagttgacctgaaccctttgtccccgggacatggcggatatgctgtattgctgccaaacggagacaaagtggatgtacacgacaagcatgcatgggatggggacaacaaaatgtggagatgggtgtacgagaagaaagatccctgtgcgttcgagctggtatccagggaagtgtgtcttttctcactgagtaggggtagtagactgagaggagcaacccctccccaaggagagctcctcacctgcccgcattcgggaaaggcatttgacctgaagggggcccgaaggattacacccatttcatgcaaaatcgacatggaatatgacttgctgtcactaccaaccggagtcatcctaggcctccacctatcagaactcgggacctcctttggcaacctctcaatgagtcttgaaatgtatgaacctgccacaactctgacccctgagcaaatcaacttctcgcttaaagagctgggaagctggaccgaggctcaactgaagagcctgtctcactcaatctgcctctccacattctccatatgggaactatcggttgggatgatcgatctaaaccctaccagggcagcaagggccttgctccatgatgataacatactggcaacattcgagaacggtcacttttccatcgtcagatgtcgtccggaaatagttcaagtcccttcgcatcctcgagcatgtcacatggatctccgcccttatgacaagcaatcacgggcatcaaccctggtggttccccttgacaacagcactgccctcctggtccccgacaacatcgtggttgaaggagtagaggccagtctatgcaaccactccgttgccatcacgctgtcgaagaacagaactcactcatacagcctctatccccagggtcgtcctgtgcttcgacagaaaggtgccgtggagctcccgacgatagggcccctccagttacatcctgccactcgagtggacctttatacactgaaagagttccaggaggaccgaatagcgcgcagtcgagtcacagacatcaaggctgccgttgacgatctgcgtgcgaagtggcgtaaaggcaaatttgaggcggacaccacgggagggggactttggtcggcgattgtgggagtcttcagttctctcggggggttcttcatgaggcccttgattgctctcgcggcgatagtgacctcaatcatcatcctgtatatccttctgcgtgtactgtgtgctgcctcatgttcgacacaccgaagagtaaggcaggactcttggtaaagaggactgcgattgttgagtggacaaaccctaggcctattccgatttagaaaaaaccagacctctcacgaggtcttttctactagctgggttttcctcattctatccagagccatggccttcgacccgaactggcagagagaaggttatgaatgggatccgtcaagtgagggcagaccgaccgatgagaacgaagacgacagaggtcatcggccaaaaacgagacttcgtacattccttgcccgcacgttaaatagccctatccgagccctattctacacaatattcctaggaattcgagcggtttgggacgggttcaaaagactcctacctgtgaggaccgaaaagggttatgcgaggttttctgagtgcgtcacatatggaatgatcggatgtgatgagtgtgtaatagacccggtgagggttgtcattgagctgaccgagatgcagttaccgattaaaggcaaaggctctacgaggttgagagcaatgataactgaagaccttctcacggggatgcgcacagccgtgcctcagatcagagtgagatcgaagatcctagcagagcggttagggagagcaatcggccgagagaccttgccggcaatgatccatcatgagtgggcatttgtgatggggaagattctcactttcatggcagacaatgtgggtatgaacgctgacacggtcgagggcgttctatcactatcagaggtcacacggcgatgggatatcggcaactctgtgtccgcagtgttcaatcctgatggccttactatcagagtagaaaacacgggttacatcatgaccagagagactgcctgcatgatcggagacattcatgctcaatttgcaatccaatacctagctgcatacctagacgaggtgatcggcacaaggacgtctctctcacccgccgaactgacctctctcaaactatggggacttaacgtcctgaaactcctaggacggaacggttatgaggtgatcgcctgcatggagcccatagggtacgctgtcctgatgatgggaagagacaggagtcctgatccctatgtcaatgacacctatttaaacagcatcctctcagaattccctgtcgactctgacgctcgagcctgcgttgaagccctcttaactatctatatgagcttcggcacaccccataaagtctcggacgcattcggcctcttcagaatgttgggacatccgatggttgatggagctgacgggattgaaaagatgcgaaggttaagcaagaaggtcaagatcccagaccagtctacagcgatcgacctcggggctatcatggccgaactgtttgtgcggagtttcgtaaagaagcacaaaaggtggcccaactgctccatcaatctcccgccacgacaccccttccaccacgcccgcctatgtgggtatgtcccggctgaaacccatcccctaaacaacactgcatcctgggcggctgtggagttcaaccaggaattcgagccgccgagacagtacaaccttgcagacatcattgatgacaagtcgtgctctcccaacaagcatgagctatatggtgcttggatgaagtcaaaaacagctgggtggcaggaacaaaagaagctcatactccgatggttcactgagaccatggttaaaccttcggagctcctggaagagattgatgcacacggcttccgagaagaggataagttgattggattaacaccaaaggagagagagctgaaattaacaccaagaatgttctccttgatgacattcaagttcagaacctaccaagtcctcactgagagtatggtcgccgatgagatcctcccgcacttcccccagatcaccatgaccatgtccaaccacgaactcacaaagaggttgattagcagaacgagacctcaatctggaggagggcgtgatgttcacatcaccgtgaacatagatttccagaaatggaacacaaacatgagacacggactggtcaaacatgtcttcgagcgactggacaacctctttggcttcaccaacttaatcagacgaactcatgaatacttccaggaggcgaaatactatctggctgaagatggaactaatctgtcgttcgacaggaacggggagttaatagatggcccatacgtttacaccggatcatacggggggaacgaggggttacgacagaagccctggacaatagttaccgtgtgtggaatatacaaggtagctagagacctgaaaatcaaacatcagatcaccggtcagggagataatcaggtggtcaccctaatatttccggatcgagagttgccttcagatccggtggagaggagcaagtactgtagagacaagagcagtcagttcctgacacgtctcagtcaatatttcgctgaggttggtttgcccgtcaagactgaagagacatggatgtcatcacgtctctatgcttacggtaagcgcatgttcttagagggagttccacttaagatgtttctcaagaagataggcagagctttcgccctctcgaatgagtttgtcccgtccctcgaggaagatctggccagagtctggagtgccaccagcgcagcggtagagcttgacctaactccctacgtaggatatgtcctcgggtgctgcttgtctgcgcaggcgatcagaaatcacctcatctactcccctgttctggagggccctctgctggttaaggcctacgagcgtaagttcattaactacgacggaggaacaaagcggggggcgatgcccggcctacgtccaacctttgagagcctagtcaaaagtatctgctggaagccaaaggccatcggagggtggccggtattgatgttagaagatctcatcatcaaagggttccctgatccggcgactagcgccctggctcaattgaagtcaatggtgccatatacctctggtatcgaccgggagatcatactttcctgtctcaaccttcccttatcgtcggtggtatctccgtcaatgttgttaaaggacccggcggccatcaacaccatcacaaccccgtccgcgggcgacatcctgcaagaggtcgccagagactatgttaccgattacccactccaaaacccgcagctcagagcagtggtcaagaacgtgaagaccgagctagacacattggccagtgacttattcaaatgtgaacctttctttcctcctttaatgagcgatatcttctcggcatctctcccggcatatcaagacaggattgttcgcaagtgctccacgacttctacaatcaggagaaaagctgccgagaggggctccgactctctcctcaaccggatgaaaaggaatgagatcaataagatgatgttacatctttgggctacctggggaaggagccctctggccagattagacaccagatgtctcacaacctgcaccaagcaattagcccaacagtatcggaaccagtcttggggaaagcagatccatggagtctcagtcggccaccccttagaactgttcggtcgaataacacccagccatagatgcctacatgaggaggaccacggagatttcctgcaaaccttcgccagcgagcatgtgaaccaagtggacaccgacatcaccacaactctggggccgttctacccttacataggctcggagacgcgagaacgggcagtcaaggttcgaaaaggagtgaattacgtagttgagccgcttctgaaacccgcagttcgactactaagagccattaattggttcattcccgaggagtcagatgcgtcccatttgctgagcaatctattagcgtctgttaccgacatcaatcctcaagaccactactcatctaccgaagtaggggggggcaacgccgtccatcgctacagctgccgactatccgacaaattgagcagagtcaacaacttatatcagttgcatacttatttatctgtcacaacagagcggttgaccaagtacagtcgaggatcaaaaaacactgacgcacacttccagagcatgatgatttatgcacaaagccgtcatatagacctcatcttggagtctctgcacaccggagagatggtaccgttggagtgtcatcatcacattgagtgcaatcactgtatagaggatatacccgacgagccaatcacgggggacccggcttggactgaagtcaagtttccttcaagtcctcaggagccctttctttacatcaggcaacaagatctgccggtcaaagacaaactcgagcctgtgcctcgcatgaacatcgtccgtcttgccggattgggtccggaggcgattagtgagctagcgcactactttgttgcattccgagttatccgggcgtcagagacggatgtcgaccctaacgatgttctctcgtggacctggctgagccgaattgatcctgacaaattggttgagtatatcgtgcatgtgttcgcttcactggaatggcatcatgtattaatgtcaggcgtgagtgtgagcgtcagagatgcattctttaagatgctagtgtctaaaagaatctcagagactccgctaagttcattctattatctggccaacctgttcgttgaccctcagactcgcgaagcactaatgagctctaaatacgggttcagcccccccgccgagacagtccccaacgcaaatgccgccgcagccgaaataagaagatgctgtgcgaacagtgcgccgtcgatcttagaatcagcccttcacagccgtgaggttgtttggatgccaggaacgaacaattatggagacgttgtcatctggtctcattacattagattacggttcagcgaagttaaactagttgacattacacgatatcagcagtggtggagacagtctgagcgagacccctacgatttggtcccggacatgcaggttcttgagagcgacctagatacgctgatgaaacggataccgaggctcatgcgcaaggcgagacgtccccctcttcaggtaattcgagaggacctggatgtcgcagtcatcaatgctgatcatcccgctcactctgtgcttcagaacaaatacaggaaattgattttcagagagccgaagattatcacgggagctgtgtacaagtacctctccctaaaatcagagttgacagagttcacctcagcaatggtgatcggagacggaactggaggtatcaccgccgccatgatggccgatgggatagatgtgtggtatcagacgctcgtcaactatgaccacgtgacacaacagggattatccgtacaagccccggcagcattggatcttctgcgcggggcaccctctggtaggctcttgaatccgggaagattcgcatcatttgggtctgacctaactgaccctcgatttacagcctactttgatcaatatcccccgttcaaggtggacactctatggtctgacgcagagggcgacttttgggacaagccttccaagttgaatcaatactttgagaacatcattgctttgagacatcggttcgtgaagacaaatggacagcttgtcgtgaaggtgtatctgactcaagacactgctaccacaattgaagcattcagaaagaagctgtccccatgcgccatcatcgtgtctctcttctcgacggaaggctccacagaatgcttcgtcctaagcaatctcatcgcaccagacacccctgtcgaccttgagatggtggagaatatccctaaactaacatcccttgttccccagaggacgacagtgaaatgctattcccgacgagtagcgtgcatcagtaaaaggtggggacttttcagatctccgagcatagcccttgaagtccaaccgttccttcactacatcacaaaggtcatctcagacaaaggaacacaactgagtctcatggcggtagctgacacaatgatcaacagttacaagaaggctatctcaccccgagtgttcgatctacaccggcatagggccgcactgggtttcgggaggagatccttgcatctcatctgggggatgatcatctcaccaatcgcttaccagcattttgagaatccggccaagttgatggatgtcctggacatgttgaccaataacatctcagctttcttatcgatatcgtcgtcaggatttgacctgtcatttagtgtcagtgcagaccgagatgtccggattgacagcaaacttgtcagactcccgctattcgaaggatcagacctaaaattcatgaaaaccatcatgtctaccctcggatctgtgttcaaccaggtcgagccttttaaggggatcgccataaacccttctaaactaatgactgtcaagaggacacaggagttacgttacaacaacctaatttacactaaggatgccatcctattccccaatgaagcggcaaaaaacactgccccgcttcgagccaacatggtataccccgtccggggagatctattcgcccctaccgatcgcataccaatcatgactctagtcagcgatgagacaacacctcagcactctcctccagaggatgaggcataactgaatcctccctgaaggctcacatgtcccacgcgacgcaagatataacgacaagcaactcgccctattaactgtgattaataaaaaaccgattattcagttgcttgagggagtttcaatccgttcagtgtatgataggaagtttctgagatggtggggattagggggcacctagagtatgtttgttcgttttatgcgtcgt

SEQ ID NO: 2 - RNA, 12423 nt, Farmington rhabdovirus
uuacgacgcauaagcugagaaacauaagagacuauguucauagucacccuguauucauuauugacuuuuaugaccuauuauucgugaggucauaugugagguaaugucaucugcuuaugcguuugcuuauaagauaaaacgauagacccuucacggguaaauccuucuccuugcaguucucgccaaguaccuccaaagucagacgauggcucguccgcuagcugcugcgcaacaucucauaaccgagcgucauucccuucaggcgacucugucgcgggcguccaagaccagagccgaggaauucgucaaagauuucuaccuucaagagcaguauucugucccgaccaucccgacggacgacauugcccagucugggcccaugcugcuucaggccauccugagcgaggaauacacaaaggccacugacauagcccaauccauccucuggaacacucccacacccaacgggcuccucagagagcaucuagaugccgaugggggaggcucauucacagcgcugcccgcgucugcaaucagacccagcgacgaggcgaaugcaugggccgcucgcaucuccgacucaggguuggggccugucuucuaugcagcccucgcugcuuacaucaucggcuggucaggaagaggagagacuagccgcgugcagcagaacauaggucagaaauggcugaugaaccugaacgcaaucuucggcaccacgaucacccauccaacaaccgugcgucugccaaucaacgucgucaacaacagccucgcagugaggaacggacuugcugccacacucuggcuauacuaccguucaucaccucagagucaggacgcguucuucuaugggcucauccgucccuguugcaguggauaucucggccugcuacaucgggugcaggagauugaugagauggagccggacuuccucagugacccccggaucauccaggugaaugaggucuacagugcacucagagcccugguucaacugggaaacgacuucaagaccgccgaugaugagcccaugcaggucugggcgugcaggggaaucaacaacggauaucugacauaucucucagaaacuccugcgaagaaaggagcuguugugcuuauguuugcccaaugcaugcugaagggcgacucugaggccuggaacagcuaccgcacugcaaccugggugaugcccuauugcgacaauguggcccuaggagcgauggcaggcuacauccaagcccgccagaacaccagggcauaugaggucucagcccagacaggucucgacgucaacauggccgcggucaaggacuuugaggccaguucaaaacccaaggcugcuccaaucucgcugaucccacgccccgcugaugucgcaucccgcaccucugagcgcccaucuauuccugagguugacagcgacgaagagcucggaggaauguaaaccaauaagcuucacugccgguaguuuaggcauacacacgcaguuccguuauccaucacacccgucccuucuuuuaugcugcuauuauuucaguugcuaagcuuccugauuugauuaacaaaaaaccguagaccuccuacgugagguauagcuagaaauugguucuaucgguugagagucuuuguacuauuagccauggaggacuauuugucuagcuuagaggccgcgagagagcucguccggacggagcuggagcccaagcguaaccucauagccagcuuagaguccgacgaucccgauccgguaauagcgccagcgguaaaaccaaaacaucccaagccaugccugagcacuaaagaagaggaucaucuccccucucuucgccuacuauucggcgcaaaacgagacaccucggugggcguagagcagacucuccacaagcgucucugcgcuugucucgacgguuaccugaccaugacgaagaaagaggccaaugccuuuaaggccgcggcugaagcagcagcauuagcagucauggacauuaagauggagcaucagcgccaggaucuagaggaucugaccgcugcuaucccuaggauagaauucaaacucaaugccauccuggaaaacaacaaggagauagccaaggcuguaacugcugcuaaggagauggagcgggagaugucguggggggaaagcgccgccagcucgcucaagucugucacccuagaugagucguuuaggggcccugaagagcuuucagagucauuuggcauccgauauaaggucagaaccuggaaugaguucaagaaggcgcuggaaaccagcauuguggaccugaggccuagcccuguuucauuuagggaauuacggacuauguggcugucucuugacaccuccuuuaggcucauuggguuugccuucauucccacaugcgagcgccuggagaccaaagccaaaugcaaggagacaaggacucuacucccccuugcagagucgaucaugcgaagaugggaccugcgggauccaaccaucuuggagaaagccugcguaguaaugaugauccgugggaaugagauugcaucgcugaaucagguaaaagauguucucccgaccacaauucguggguggaagaucgcuuauuagucacugcucccauuagucccacuagacggcauacuuccauuccgcccuuuaauuccccugucagacacucaugcuccgaaaucacuaaccauccuuguccaccaagcaauacgcauauucaguagcacugcaucucgcccucccccuaucaagccccagcgcugcagaucuucaccacauauauacaugcaucaacuacaugugauuuagaaaaaaccagacccuucacggguaauagccuaacucacgaacguuccucucguuucguaugauaaggccuuaagcauugucgauacggucguuaugcgucgguucuuuuuaggagagagcagugccccugcgagggacugggaguccgagcgaccuccccccuaugcuguugaggucccucaaagucacgggauaagagucaccggguacuuccagugcaacgagcguccgaaauccaagaagacccuccacagcuucgccguaaaacucugcgacgcaauuaagccgguucgagcggaugcucccagcuugaagauagcaauauggacggcucuagaucuggccuucgugaaaccucccaauggaacuguaacaauagaugcggcggugaaagcuacaccgcuaaucgggaacacccaguacaccguaggcgaugaaaucuuccagaugcuagggagaagggguggccugaucgucaucaggaacuuaccccaugauuauccucgaacguugauugaguucgccucucccgagccuugagcaccagggcaucgguccgcccgcccugugaucucccguagccgggcucagcgaucaagccggcccgggucgggggggacuggugcaacacaaggggcggcaguggacgcugauuaacaaaaaaccaccuauauagaccccucacggucuuagacucuguugccagcugacaaccaacacacaagacaucucucugauucagccgacccgaucgauuccuccccacccaauuccuaccaacgcacuccucacaagcuccaccaugcucaggauccagaucccuccgauugcuaucauucugguaagucuccucacacucgaccuguccggugcaaggaggacaaccacacaaagaaucccucuccuuaaugauucgugggauuuguucucgagcuauggcgacauucccgaagaacuugucguauaccagaacuacagccacaauuccuccgaguuacccccuccuggcuucgagagaugguacauaaaccgaagaguggcagacacuuccauaccgugcaggggccccugucuagugcccuacauccuucauggccucaaugacacaacugucucucgacggggaggaggauggcgaagguccggaaugaaguacccaacccacgcugucaggcuaggcccuucaacagacgacgagagaguugaggaagacaucggcuacgucaaugucuccgcacuauccugcacagggucgcccguugagauggcgauaccaacaauccccgacugcaccagugcuauccauccacgauccgagguuacugugcccgucaagcucgaugucaugagacgaaaucccaacuacccucccauuagagcguggucgugcaucggacagaaaaucaccaaccgaugugauugggcacucuucggcgagaaccucauauauacucaaguugaagcuagcucucuagcauucaagcacacaagagccucucuuuugaacgaauccaacgggauagacgcugaaggacgugcaguucccuauauccucggggauaucgaacccggguacugccgaacccuauucaacacaugggucucuagugagaucgugucaugcacgcccaucgaacuuguccuaguugaccugaacccuuuguccccgggacauggcggauaugcuguauugcugccaaacggagacaaaguggauguacacgacaagcaugcaugggauggggacaacaaaauguggagauggguguacgagaagaaagaucccugugcguucgagcugguauccagggaagugugucuuuucucacugaguagggguaguagacugagaggagcaaccccuccccaaggagagcuccucaccugcccgcauucgggaaaggcauuugaccugaagggggcccgaaggauuacacccauuucaugcaaaaucgacauggaauaugacuugcugucacuaccaaccggagucauccuaggccuccaccuaucagaacucgggaccuccuuuggcaaccucucaaugagucuugaaauguaugaaccugccacaacucugaccccugagcaaaucaacuucucgcuuaaagagcugggaagcuggaccgaggcucaacugaagagccugucucacucaaucugccucuccacauucuccauaugggaacuaucgguugggaugaucgaucuaaacccuaccagggcagcaagggccuugcuccaugaugauaacauacuggcaacauucgagaacggucacuuuuccaucgucagaugucguccggaaauaguucaagucccuucgcauccucgagcaugucacauggaucuccgcccuuaugacaagcaaucacgggcaucaacccuggugguuccccuugacaacagcacugcccuccugguccccgacaacaucgugguugaaggaguagaggccagucuaugcaaccacuccguugccaucacgcugucgaagaacagaacucacucauacagccucuauccccagggucguccugugcuucgacagaaaggugccguggagcucccgacgauagggccccuccaguuacauccugccacucgaguggaccuuuauacacugaaagaguuccaggaggaccgaauagcgcgcagucgagucacagacaucaaggcugccguugacgaucugcgugcgaaguggcguaaaggcaaauuugaggcggacaccacgggagggggacuuuggucggcgauugugggagucuucaguucucucgggggguucuucaugaggcccuugauugcucucgcggcgauagugaccucaaucaucauccuguauauccuucugcguguacugugugcugccucauguucgacacaccgaagaguaaggcaggacucuugguaaagaggacugcgauuguugaguggacaaacccuaggccuauuccgauuuagaaaaaaccagaccucucacgaggucuuuucuacuagcuggguuuuccucauucuauccagagccauggccuucgacccgaacuggcagagagaagguuaugaaugggauccgucaagugagggcagaccgaccgaugagaacgaagacgacagaggucaucggccaaaaacgagacuucguacauuccuugcccgcacguuaaauagcccuauccgagcccuauucuacacaauauuccuaggaauucgagcgguuugggacggguucaaaagacuccuaccugugaggaccgaaaaggguuaugcgagguuuucugagugcgucacauauggaaugaucggaugugaugaguguguaauagacccggugaggguugucauugagcugaccgagaugcaguuaccgauuaaaggcaaaggcucuacgagguugagagcaaugauaacugaagaccuucucacggggaugcgcacagccgugccucagaucagagugagaucgaagauccuagcagagcgguuagggagagcaaucggccgagagaccuugccggcaaugauccaucaugagugggcauuugugauggggaagauucucacuuucauggcagacaauguggguaugaacgcugacacggucgagggcguucuaucacuaucagaggucacacggcgaugggauaucggcaacucuguguccgcaguguucaauccugauggccuuacuaucagaguagaaaacacggguuacaucaugaccagagagacugccugcaugaucggagacauucaugcucaauuugcaauccaauaccuagcugcauaccuagacgaggugaucggcacaaggacgucucucucacccgccgaacugaccucucucaaacuauggggacuuaacguccugaaacuccuaggacggaacgguuaugaggugaucgccugcauggagcccauaggguacgcuguccugaugaugggaagagacaggaguccugaucccuaugucaaugacaccuauuuaaacagcauccucucagaauucccugucgacucugacgcucgagccugcguugaagcccucuuaacuaucuauaugagcuucggcacaccccauaaagucucggacgcauucggccucuucagaauguugggacauccgaugguugauggagcugacgggauugaaaagaugcgaagguuaagcaagaaggucaagaucccagaccagucuacagcgaucgaccucggggcuaucauggccgaacuguuugugcggaguuucguaaagaagcacaaaagguggcccaacugcuccaucaaucucccgccacgacaccccuuccaccacgcccgccuauguggguaugucccggcugaaacccauccccuaaacaacacugcauccugggcggcuguggaguucaaccaggaauucgagccgccgagacaguacaaccuugcagacaucauugaugacaagucgugcucucccaacaagcaugagcuauauggugcuuggaugaagucaaaaacagcuggguggcaggaacaaaagaagcucauacuccgaugguucacugagaccaugguuaaaccuucggagcuccuggaagagauugaugcacacggcuuccgagaagaggauaaguugauuggauuaacaccaaaggagagagagcugaaauuaacaccaagaauguucuccuugaugacauucaaguucagaaccuaccaaguccucacugagaguauggucgccgaugagauccucccgcacuucccccagaucaccaugaccauguccaaccacgaacucacaaagagguugauuagcagaacgagaccucaaucuggaggagggcgugauguucacaucaccgugaacauagauuuccagaaauggaacacaaacaugagacacggacuggucaaacaugucuucgagcgacuggacaaccucuuuggcuucaccaacuuaaucagacgaacucaugaauacuuccaggaggcgaaauacuaucuggcugaagauggaacuaaucugucguucgacaggaacggggaguuaauagauggcccauacguuuacaccggaucauacggggggaacgagggguuacgacagaagcccuggacaauaguuaccguguguggaauauacaagguagcuagagaccugaaaaucaaacaucagaucaccggucagggagauaaucagguggucacccuaauauuuccggaucgagaguugccuucagauccgguggagaggagcaaguacuguagagacaagagcagucaguuccugacacgucucagucaauauuucgcugagguugguuugcccgucaagacugaagagacauggaugucaucacgucucuaugcuuacgguaagcgcauguucuuagagggaguuccacuuaagauguuucucaagaagauaggcagagcuuucgcccucucgaaugaguuugucccgucccucgaggaagaucuggccagagucuggagugccaccagcgcagcgguagagcuugaccuaacucccuacguaggauauguccucgggugcugcuugucugcgcaggcgaucagaaaucaccucaucuacuccccuguucuggagggcccucugcugguuaaggccuacgagcguaaguucauuaacuacgacggaggaacaaagcggggggcgaugcccggccuacguccaaccuuugagagccuagucaaaaguaucugcuggaagccaaaggccaucggaggguggccgguauugauguuagaagaucucaucaucaaaggguucccugauccggcgacuagcgcccuggcucaauugaagucaauggugccauauaccucugguaucgaccgggagaucauacuuuccugucucaaccuucccuuaucgucggugguaucuccgucaauguuguuaaaggacccggcggccaucaacaccaucacaaccccguccgcgggcgacauccugcaagaggucgccagagacuauguuaccgauuacccacuccaaaacccgcagcucagagcaguggucaagaacgugaagaccgagcuagacacauuggccagugacuuauucaaaugugaaccuuucuuuccuccuuuaaugagcgauaucuucucggcaucucucccggcauaucaagacaggauuguucgcaagugcuccacgacuucuacaaucaggagaaaagcugccgagaggggcuccgacucucuccucaaccggaugaaaaggaaugagaucaauaagaugauguuacaucuuugggcuaccuggggaaggagcccucuggccagauuagacaccagaugucucacaaccugcaccaagcaauuagcccaacaguaucggaaccagucuuggggaaagcagauccauggagucucagucggccaccccuuagaacuguucggucgaauaacacccagccauagaugccuacaugaggaggaccacggagauuuccugcaaaccuucgccagcgagcaugugaaccaaguggacaccgacaucaccacaacucuggggccguucuacccuuacauaggcucggagacgcgagaacgggcagucaagguucgaaaaggagugaauuacguaguugagccgcuucugaaacccgcaguucgacuacuaagagccauuaauugguucauucccgaggagucagaugcgucccauuugcugagcaaucuauuagcgucuguuaccgacaucaauccucaagaccacuacucaucuaccgaaguaggggggggcaacgccguccaucgcuacagcugccgacuauccgacaaauugagcagagucaacaacuuauaucaguugcauacuuauuuaucugucacaacagagcgguugaccaaguacagucgaggaucaaaaaacacugacgcacacuuccagagcaugaugauuuaugcacaaagccgucauauagaccucaucuuggagucucugcacaccggagagaugguaccguuggagugucaucaucacauugagugcaaucacuguauagaggauauacccgacgagccaaucacgggggacccggcuuggacugaagucaaguuuccuucaaguccucaggagcccuuucuuuacaucaggcaacaagaucugccggucaaagacaaacucgagccugugccucgcaugaacaucguccgucuugccggauuggguccggaggcgauuagugagcuagcgcacuacuuuguugcauuccgaguuauccgggcgucagagacggaugucgacccuaacgauguucucucguggaccuggcugagccgaauugauccugacaaauugguugaguauaucgugcauguguucgcuucacuggaauggcaucauguauuaaugucaggcgugagugugagcgucagagaugcauucuuuaagaugcuagugucuaaaagaaucucagagacuccgcuaaguucauucuauuaucuggccaaccuguucguugacccucagacucgcgaagcacuaaugagcucuaaauacggguucagcccccccgccgagacaguccccaacgcaaaugccgccgcagccgaaauaagaagaugcugugcgaacagugcgccgucgaucuuagaaucagcccuucacagccgugagguuguuuggaugccaggaacgaacaauuauggagacguugucaucuggucucauuacauuagauuacgguucagcgaaguuaaacuaguugacauuacacgauaucagcagugguggagacagucugagcgagaccccuacgauuuggucccggacaugcagguucuugagagcgaccuagauacgcugaugaaacggauaccgaggcucaugcgcaaggcgagacgucccccucuucagguaauucgagaggaccuggaugucgcagucaucaaugcugaucaucccgcucacucugugcuucagaacaaauacaggaaauugauuuucagagagccgaagauuaucacgggagcuguguacaaguaccucucccuaaaaucagaguugacagaguucaccucagcaauggugaucggagacggaacuggagguaucaccgccgccaugauggccgaugggauagaugugugguaucagacgcucgucaacuaugaccacgugacacaacagggauuauccguacaagccccggcagcauuggaucuucugcgcggggcacccucugguaggcucuugaauccgggaagauucgcaucauuugggucugaccuaacugacccucgauuuacagccuacuuugaucaauaucccccguucaagguggacacucuauggucugacgcagagggcgacuuuugggacaagccuuccaaguugaaucaauacuuugagaacaucauugcuuugagacaucgguucgugaagacaaauggacagcuugucgugaagguguaucugacucaagacacugcuaccacaauugaagcauucagaaagaagcuguccccaugcgccaucaucgugucucucuucucgacggaaggcuccacagaaugcuucguccuaagcaaucucaucgcaccagacaccccugucgaccuugagaugguggagaauaucccuaaacuaacaucccuuguuccccagaggacgacagugaaaugcuauucccgacgaguagcgugcaucaguaaaagguggggacuuuucagaucuccgagcauagcccuugaaguccaaccguuccuucacuacaucacaaaggucaucucagacaaaggaacacaacugagucucauggcgguagcugacacaaugaucaacaguuacaagaaggcuaucucaccccgaguguucgaucuacaccggcauagggccgcacuggguuucgggaggagauccuugcaucucaucugggggaugaucaucucaccaaucgcuuaccagcauuuugagaauccggccaaguugauggauguccuggacauguugaccaauaacaucucagcuuucuuaucgauaucgucgucaggauuugaccugucauuuagugucagugcagaccgagauguccggauugacagcaaacuugucagacucccgcuauucgaaggaucagaccuaaaauucaugaaaaccaucaugucuacccucggaucuguguucaaccaggucgagccuuuuaaggggaucgccauaaacccuucuaaacuaaugacugucaagaggacacaggaguuacguuacaacaaccuaauuuacacuaaggaugccauccuauuccccaaugaagcggcaaaaaacacugccccgcuucgagccaacaugguauaccccguccggggagaucuauucgccccuaccgaucgcauaccaaucaugacucuagucagcgaugagacaacaccucagcacucuccuccagaggaugaggcauaacugaauccucccugaaggcucacaugucccacgcgacgcaagauauaacgacaagcaacucgcccuauuaacugugauuaauaaaaaaccgauuauucaguugcuugagggaguuucaauccguucaguguaugauaggaaguuucugagaugguggggauuagggggcaccuagaguauguuuguucguuuuaugcgucgu

SEQ ID NO: 3 - protein, 412 aa, Farmington rhabdovirus
MARPLAAAQHLITERHSLQATLSRASKTRAEEFVKDFYLQEQYSVPTIPTDDIAQSGPMLLQAILSEEYTKATDIAQSILWNTPTPNGLLREHLDADGGGSFTALPASAIRPSDEANAWAARISDSGLGPVFYAALAAYIIGWSGRGETSRVQQNIGQKWLMNLNAIFGTTITHPTTVRLPINVVNNSLAVRNGLAATLWLYYRSSPQSQDAFFYGLIRPCCSGYLGLLHRVQEIDEMEPDFLSDPRIIQVNEVYSALRALVQLGNDFKTADDEPMQVWACRGINNGYLTYLSETPAKKGAVVLMFAQCMLKGDSEAWNSYRTATWVMPYCDNVALGAMAGYIQARQNTRAYEVSAQTGLDVNMAAVKDFEASSKPKAAPISLIPRPADVASRTSERPSIPEVDSDEELGGM

SEQ ID NO: 4 - protein, 316 aa, Farmington rhabdovirus
MEDYLSSLEAARELVRTELEPKRNLIASLESDDPDPVIAPAVKPKHPKPCLSTKEEDHLPSLRLLFGAKRDTSVGVEQTLHKRLCACLDGYLTMTKKEANAFKAAAEAAALAVMDIKMEHQRQDLEDLTAAIPRIEFKLNAILENNKEIAKAVTAAKEMEREMSWGESAASSLKSVTLDESFRGPEELSESFGIRYKVRTWNEFKKALETSIVDLRPSPVSFRELRTMWLSLDTSFRLIGFAFIPTCERLETKAKCKETRTLLPLAESIMRRWDLRDPTILEKACVVMMIRGNEIASLNQVKDVLPTTIRGWKIAY

SEQ ID NO: 5 - protein, 148 aa, Farmington rhabdovirus
MRRFFLGESSAPARDWESERPPPYAVEVPQSHGIRVTGYFQCNERPKSKKTLHSFAVKLCDAIKPVRADAPSLKIAIWTALDLAFVKPPNGTVTIDAAVKATPLIGNTQYTVGDEIFQMLGRRGGLIVIRNLPHDYPRTLIEFASPEP

SEQ ID NO: 6 - protein, 704 aa, Farmington rhabdovirus
MLRIQIPPIAIILVSLLTLDLSGARRTTTQRIPLLNDSWDLFSSYGDIPEELVVYQNYSHNSSELPPPGFERWYINRRVADTSIPCRGPCLVPYILHGLNDTTVSRRGGGWRRSGMKYPTHAVRLGPSTDDERVEEDIGYVNVSALSCTGSPVEMAIPTIPDCTSAIHPRSEVTVPVKLDVMRRNPNYPPIRAWSCIGQKITNRCDWALFGENLIYTQVEASSLAFKHTRASLLNESNGIDAEGRAVPYILGDIEPGYCRTLFNTWVSSEIVSCTPIELVLVDLNPLSPGHGGYAVLLPNGDKVDVHDKHAWDGDNKMWRWVYEKKDPCAFELVSREVCLFSLSRGSRLRGATPPQGELLTCPHSGKAFDLKGARRITPISCKIDMEYDLLSLPTGVILGLHLSELGTSFGNLSMSLEMYEPATTLTPEQINFSLKELGSWTEAQLKSLSHSICLSTFSIWELSVGMIDLNPTRAARALLHDDNILATFENGHFSIVRCRPEIVQVPSHPRACHMDLRPYDKQSRASTLVVPLDNSTALLVPDNIVVEGVEASLCNHSVAITLSKNRTHSYSLYPQGRPVLRQKGAVELPTIGPLQLHPATRVDLYTLKEFQEDRIARSRVTDIKAAVDDLRAKWRKGKFEADTTGGGLWSAIVGVFSSLGGFFMRPLIALAAIVTSIIILYILLRVLCAASCSTHRRVRQDSW

SEQ ID NO: 7 - protein, 2129 aa, Farmington rhabdovirus
MAFDPNWQREGYEWDPSSEGRPTDENEDDRGHRPKTRLRTFLARTLNSPIRALFYTIFLGIRAVWDGFKRLLPVRTEKGYARFSECVTYGMIGCDECVIDPVRVVIELTEMQLPIKGKGSTRLRAMITEDLLTGMRTAVPQIRVRSKILAERLGRAIGRETLPAMIHHEWAFVMGKILTFMADNVGMNADTVEGVLSLSEVTRRWDIGNSVSAVFNPDGLTIRVENTGYIMTRETACMIGDIHAQFAIQYLAAYLDEVIGTRTSLSPAELTSLKLWGLNVLKLLGRNGYEVIACMEPIGYAVLMMGRDRSPDPYVNDTYLNSILSEFPVDSDARACVEALLTIYMSFGTPHKVSDAFGLFRMLGHPMVDGADGIEKMRRLSKKVKIPDQSTAIDLGAIMAELFVRSFVKKHKRWPNCSINLPPRHPFHHARLCGYVPAETHPLNNTASWAAVEFNQEFEPPRQYNLADIIDDKSCSPNKHELYGAWMKSKTAGWQEQKKLILRWFTETMVKPSELLEEIDAHGFREEDKLIGLTPKERELKLTPRMFSLMTFKFRTYQVLTESMVADEILPHFPQITMTMSNHELTKRLISRTRPQSGGGRDVHITVNIDFQKWNTNMRHGLVKHVFERLDNLFGFTNLIRRTHEYFQEAKYYLAEDGTNLSFDRNGELIDGPYVYTGSYGGNEGLRQKPWTIVTVCGIYKVARDLKIKHQITGQGDNQVVTLIFPDRELPSDPVERSKYCRDKSSQFLTRLSQYFAEVGLPVKTEETWMSSRLYAYGKRMFLEGVPLKMFLKKIGRAFALSNEFVPSLEEDLARVWSATSAAVELDLTPYVGYVLGCCLSAQAIRNHLIYSPVLEGPLLVKAYERKFINYDGGTKRGAMPGLRPTFESLVKSICWKPKAIGGWPVLMLEDLIIKGFPDPATSALAQLKSMVPYTSGIDREIILSCLNLPLSSVVSPSMLLKDPAAINTITTPSAGDILQEVARDYVTDYPLQNPQLRAVVKNVKTELDTLASDLFKCEPFFPPLMSDIFSASLPAYQDRIVRKCSTTSTIRRKAAERGSDSLLNRMKRNEINKMMLHLWATWGRSPLARLDTRCLTTCTKQLAQQYRNQSWGKQIHGVSVGHPLELFGRITPSHRCLHEEDHGDFLQTFASEHVNQVDTDITTTLGPFYPYIGSETRERAVKVRKGVNYVVEPLLKPAVRLLRAINWFIPEESDASHLLSNLLASVTDINPQDHYSSTEVGGGNAVHRYSCRLSDKLSRVNNLYQLHTYLSVTTERLTKYSRGSKNTDAHFQSMMIYAQSRHIDLILESLHTGEMVPLECHHHIECNHCIEDIPDEPITGDPAWTEVKFPSSPQEPFLYIRQQDLPVKDKLEPVPRMNIVRLAGLGPEAISELAHYFVAFRVIRASETDVDPNDVLSWTWLSRIDPDKLVEYIVHVFASLEWHHVLMSGVSVSVRDAFFKMLVSKRISETPLSSFYYLANLFVDPQTREALMSSKYGFSPPAETVPNANAAAAEIRRCCANSAPSILESALHSREVVWMPGTNNYGDVVIWSHYIRLRFSEVKLVDITRYQQWWRQSERDPYDLVPDMQVLESDLDTLMKRIPRLMRKARRPPLQVIREDLDVAVINADHPAHSVLQNKYRKLIFREPKIITGAVYKYLSLKSELTEFTSAMVIGDGTGGITAAMMADGIDVWYQTLVNYDHVTQQGLSVQAPAALDLLRGAPSGRLLNPGRFASFGSDLTDPRFTAYFDQYPPFKVDTLWSDAEGDFWDKPSKLNQYFENIIALRHRFVKTNGQLVVKVYLTQDTATTIEAFRKKLSPCAIIVSLFSTEGSTECFVLSNLIAPDTPVDLEMVENIPKLTSLVPQRTTVKCYSRRVACISKRWGLFRSPSIALEVQPFLHYITKVISDKGTQLSLMAVADTMINSYKKAISPRVFDLHRHRAALGFGRRSLHLIWGMIISPIAYQHFENPAKLMDVLDMLTNNISAFLSISSSGFDLSFSVSADRDVRIDSKLVRLPLFEGSDLKFMKTIMSTLGSVFNQVEPFKGIAINPSKLMTVKRTQELRYNNLIYTKDAILFPNEAAKNTAPLRANMVYPVRGDLFAPTDRIPIMTLVSDETTPQHSPPEDEA

SEQ ID NO: 8 - DNA, 1236 bp, Farmington rhabdovirus
atggctcgtccgctagctgctgcgcaacatctcataaccgagcgtcattcccttcaggcgactctgtcgcgggcgtccaagaccagagccgaggaattcgtcaaagatttctaccttcaagagcagtattctgtcccgaccatcccgacggacgacattgcccagtctgggcccatgctgcttcaggccatcctgagcgaggaatacacaaaggccactgacatagcccaatccatcctctggaacactcccacacccaacgggctcctcagagagcatctagatgccgatgggggaggctcattcacagcgctgcccgcgtctgcaatcagacccagcgacgaggcgaatgcatgggccgctcgcatctccgactcagggttggggcctgtcttctatgcagccctcgctgcttacatcatcggctggtcaggaagaggagagactagccgcgtgcagcagaacataggtcagaaatggctgatgaacctgaacgcaatcttcggcaccacgatcacccatccaacaaccgtgcgtctgccaatcaacgtcgtcaacaacagcctcgcagtgaggaacggacttgctgccacactctggctatactaccgttcatcacctcagagtcaggacgcgttcttctatgggctcatccgtccctgttgcagtggatatctcggcctgctacatcgggtgcaggagattgatgagatggagccggacttcctcagtgacccccggatcatccaggtgaatgaggtctacagtgcactcagagccctggttcaactgggaaacgacttcaagaccgccgatgatgagcccatgcaggtctgggcgtgcaggggaatcaacaacggatatctgacatatctctcagaaactcctgcgaagaaaggagctgttgtgcttatgtttgcccaatgcatgctgaagggcgactctgaggcctggaacagctaccgcactgcaacctgggtgatgccctattgcgacaatgtggccctaggagcgatggcaggctacatccaagcccgccagaacaccagggcatatgaggtctcagcccagacaggtctcgacgtcaacatggccgcggtcaaggactttgaggccagttcaaaacccaaggctgctccaatctcgctgatcccacgccccgctgatgtcgcatcccgcacctctgagcgcccatctattcctgaggttgacagcgacgaagagctcggaggaatg

SEQ ID NO: 9 - DNA, 948 bp, Farmington rhabdovirus
atggaggactatttgtctagcttagaggccgcgagagagctcgtccggacggagctggagcccaagcgtaacctcatagccagcttagagtccgacgatcccgatccggtaatagcgccagcggtaaaaccaaaacatcccaagccatgcctgagcactaaagaagaggatcatctcccctctcttcgcctactattcggcgcaaaacgagacacctcggtgggcgtagagcagactctccacaagcgtctctgcgcttgtctcgacggttacctgaccatgacgaagaaagaggccaatgcctttaaggccgcggctgaagcagcagcattagcagtcatggacattaagatggagcatcagcgccaggatctagaggatctgaccgctgctatccctaggatagaattcaaactcaatgccatcctggaaaacaacaaggagatagccaaggctgtaactgctgctaaggagatggagcgggagatgtcgtggggggaaagcgccgccagctcgctcaagtctgtcaccctagatgagtcgtttaggggccctgaagagctttcagagtcatttggcatccgatataaggtcagaacctggaatgagttcaagaaggcgctggaaaccagcattgtggacctgaggcctagccctgtttcatttagggaattacggactatgtggctgtctcttgacacctcctttaggctcattgggtttgccttcattcccacatgcgagcgcctggagaccaaagccaaatgcaaggagacaaggactctactcccccttgcagagtcgatcatgcgaagatgggacctgcgggatccaaccatcttggagaaagcctgcgtagtaatgatgatccgtgggaatgagattgcatcgctgaatcaggtaaaagatgttctcccgaccacaattcgtgggtggaagatcgcttat

SEQ ID NO: 10 - DNA, 444 bp, Farmington rhabdovirus
atgcgtcggttctttttaggagagagcagtgcccctgcgagggactgggagtccgagcgacctcccccctatgctgttgaggtccctcaaagtcacgggataagagtcaccgggtacttccagtgcaacgagcgtccgaaatccaagaagaccctccacagcttcgccgtaaaactctgcgacgcaattaagccggttcgagcggatgctcccagcttgaagatagcaatatggacggctctagatctggccttcgtgaaacctcccaatggaactgtaacaatagatgcggcggtgaaagctacaccgctaatcgggaacacccagtacaccgtaggcgatgaaatcttccagatgctagggagaaggggtggcctgatcgtcatcaggaacttaccccatgattatcctcgaacgttgattgagttcgcctctcccgagcct

SEQ ID NO: 11 - DNA, 2112 bp, Farmington rhabdovirus
atgctcaggatccagatccctccgattgctatcattctggtaagtctcctcacactcgacctgtccggtgcaaggaggacaaccacacaaagaatccctctccttaatgattcgtgggatttgttctcgagctatggcgacattcccgaagaacttgtcgtataccagaactacagccacaattcctccgagttaccccctcctggcttcgagagatggtacataaaccgaagagtggcagacacttccataccgtgcaggggcccctgtctagtgccctacatccttcatggcctcaatgacacaactgtctctcgacggggaggaggatggcgaaggtccggaatgaagtacccaacccacgctgtcaggctaggcccttcaacagacgacgagagagttgaggaagacatcggctacgtcaatgtctccgcactatcctgcacagggtcgcccgttgagatggcgataccaacaatccccgactgcaccagtgctatccatccacgatccgaggttactgtgcccgtcaagctcgatgtcatgagacgaaatcccaactaccctcccattagagcgtggtcgtgcatcggacagaaaatcaccaaccgatgtgattgggcactcttcggcgagaacctcatatatactcaagttgaagctagctctctagcattcaagcacacaagagcctctcttttgaacgaatccaacgggatagacgctgaaggacgtgcagttccctatatcctcggggatatcgaacccgggtactgccgaaccctattcaacacatgggtctctagtgagatcgtgtcatgcacgcccatcgaacttgtcctagttgacctgaaccctttgtccccgggacatggcggatatgctgtattgctgccaaacggagacaaagtggatgtacacgacaagcatgcatgggatggggacaacaaaatgtggagatgggtgtacgagaagaaagatccctgtgcgttcgagctggtatccagggaagtgtgtcttttctcactgagtaggggtagtagactgagaggagcaacccctccccaaggagagctcctcacctgcccgcattcgggaaaggcatttgacctgaagggggcccgaaggattacacccatttcatgcaaaatcgacatggaatatgacttgctgtcactaccaaccggagtcatcctaggcctccacctatcagaactcgggacctcctttggcaacctctcaatgagtcttgaaatgtatgaacctgccacaactctgacccctgagcaaatcaacttctcgcttaaagagctgggaagctggaccgaggctcaactgaagagcctgtctcactcaatctgcctctccacattctccatatgggaactatcggttgggatgatcgatctaaaccctaccagggcagcaagggccttgctccatgatgataacatactggcaacattcgagaacggtcacttttccatcgtcagatgtcgtccggaaatagttcaagtcccttcgcatcctcgagcatgtcacatggatctccgcccttatgacaagcaatcacgggcatcaaccctggtggttccccttgacaacagcactgccctcctggtccccgacaacatcgtggttgaaggagtagaggccagtctatgcaaccactccgttgccatcacgctgtcgaagaacagaactcactcatacagcctctatccccagggtcgtcctgtgcttcgacagaaaggtgccgtggagctcccgacgatagggcccctccagttacatcctgccactcgagtggacctttatacactgaaagagttccaggaggaccgaatagcgcgcagtcgagtcacagacatcaaggctgccgttgacgatctgcgtgcgaagtggcgtaaaggcaaatttgaggcggacaccacgggagggggactttggtcggcgattgtgggagtcttcagttctctcggggggttcttcatgaggcccttgattgctctcgcggcgatagtgacctcaatcatcatcctgtatatccttctgcgtgtactgtgtgctgcctcatgttcgacacaccgaagagtaaggcaggactcttgg

SEQ ID NO: 12 - DNA, 6387 bp, Farmington rhabdovirus
atggccttcgacccgaactggcagagagaaggttatgaatgggatccgtcaagtgagggcagaccgaccgatgagaacgaagacgacagaggtcatcggccaaaaacgagacttcgtacattccttgcccgcacgttaaatagccctatccgagccctattctacacaatattcctaggaattcgagcggtttgggacgggttcaaaagactcctacctgtgaggaccgaaaagggttatgcgaggttttctgagtgcgtcacatatggaatgatcggatgtgatgagtgtgtaatagacccggtgagggttgtcattgagctgaccgagatgcagttaccgattaaaggcaaaggctctacgaggttgagagcaatgataactgaagaccttctcacggggatgcgcacagccgtgcctcagatcagagtgagatcgaagatcctagcagagcggttagggagagcaatcggccgagagaccttgccggcaatgatccatcatgagtgggcatttgtgatggggaagattctcactttcatggcagacaatgtgggtatgaacgctgacacggtcgagggcgttctatcactatcagaggtcacacggcgatgggatatcggcaactctgtgtccgcagtgttcaatcctgatggccttactatcagagtagaaaacacgggttacatcatgaccagagagactgcctgcatgatcggagacattcatgctcaatttgcaatccaatacctagctgcatacctagacgaggtgatcggcacaaggacgtctctctcacccgccgaactgacctctctcaaactatggggacttaacgtcctgaaactcctaggacggaacggttatgaggtgatcgcctgcatggagcccatagggtacgctgtcctgatgatgggaagagacaggagtcctgatccctatgtcaatgacacctatttaaacagcatcctctcagaattccctgtcgactctgacgctcgagcctgcgttgaagccctcttaactatctatatgagcttcggcacaccccataaagtctcggacgcattcggcctcttcagaatgttgggacatccgatggttgatggagctgacgggattgaaaagatgcgaaggttaagcaagaaggtcaagatcccagaccagtctacagcgatcgacctcggggctatcatggccgaactgtttgtgcggagtttcgtaaagaagcacaaaaggtggcccaactgctccatcaatctcccgccacgacaccccttccaccacgcccgcctatgtgggtatgtcccggctgaaacccatcccctaaacaacactgcatcctgggcggctgtggagttcaaccaggaattcgagccgccgagacagtacaaccttgcagacatcattgatgacaagtcgtgctctcccaacaagcatgagctatatggtgcttggatgaagtcaaaaacagctgggtggcaggaacaaaagaagctcatactccgatggttcactgagaccatggttaaaccttcggagctcctggaagagattgatgcacacggcttccgagaagaggataagttgattggattaacaccaaaggagagagagctgaaattaacaccaagaatgttctccttgatgacattcaagttcagaacctaccaagtcctcactgagagtatggtcgccgatgagatcctcccgcacttcccccagatcaccatgaccatgtccaaccacgaactcacaaagaggttgattagcagaacgagacctcaatctggaggagggcgtgatgttcacatcaccgtgaacatagatttccagaaatggaacacaaacatgagacacggactggtcaaacatgtcttcgagcgactggacaacctctttggcttcaccaacttaatcagacgaactcatgaatacttccaggaggcgaaatactatctggctgaagatggaactaatctgtcgttcgacaggaacggggagttaatagatggcccatacgtttacaccggatcatacggggggaacgaggggttacgacagaagccctggacaatagttaccgtgtgtggaatatacaaggtagctagagacctgaaaatcaaacatcagatcaccggtcagggagataatcaggtggtcaccctaatatttccggatcgagagttgccttcagatccggtggagaggagcaagtactgtagagacaagagcagtcagttcctgacacgtctcagtcaatatttcgctgaggttggtttgcccgtcaagactgaagagacatggatgtcatcacgtctctatgcttacggtaagcgcatgttcttagagggagttccacttaagatgtttctcaagaagataggcagagctttcgccctctcgaatgagtttgtcccgtccctcgaggaagatctggccagagtctggagtgccaccagcgcagcggtagagcttgacctaactccctacgtaggatatgtcctcgggtgctgcttgtctgcgcaggcgatcagaaatcacctcatctactcccctgttctggagggccctctgctggttaaggcctacgagcgtaagttcattaactacgacggaggaacaaagcggggggcgatgcccggcctacgtccaacctttgagagcctagtcaaaagtatctgctggaagccaaaggccatcggagggtggccggtattgatgttagaagatctcatcatcaaagggttccctgatccggcgactagcgccctggctcaattgaagtcaatggtgccatatacctctggtatcgaccgggagatcatactttcctgtctcaaccttcccttatcgtcggtggtatctccgtcaatgttgttaaaggacccggcggccatcaacaccatcacaaccccgtccgcgggcgacatcctgcaagaggtcgccagagactatgttaccgattacccactccaaaacccgcagctcagagcagtggtcaagaacgtgaagaccgagctagacacattggccagtgacttattcaaatgtgaacctttctttcctcctttaatgagcgatatcttctcggcatctctcccggcatatcaagacaggattgttcgcaagtgctccacgacttctacaatcaggagaaaagctgccgagaggggctccgactctctcctcaaccggatgaaaaggaatgagatcaataagatgatgttacatctttgggctacctggggaaggagccctctggccagattagacaccagatgtctcacaacctgcaccaagcaattagcccaacagtatcggaaccagtcttggggaaagcagatccatggagtctcagtcggccaccccttagaactgttcggtcgaataacacccagccatagatgcctacatgaggaggaccacggagatttcctgcaaaccttcgccagcgagcatgtgaaccaagtggacaccgacatcaccacaactctggggccgttctacccttacataggctcggagacgcgagaacgggcagtcaaggttcgaaaaggagtgaattacgtagttgagccgcttctgaaacccgcagttcgactactaagagccattaattggttcattcccgaggagtcagatgcgtcccatttgctgagcaatctattagcgtctgttaccgacatcaatcctcaagaccactactcatctaccgaagtaggggggggcaacgccgtccatcgctacagctgccgactatccgacaaattgagcagagtcaacaacttatatcagttgcatacttatttatctgtcacaacagagcggttgaccaagtacagtcgaggatcaaaaaacactgacgcacacttccagagcatgatgatttatgcacaaagccgtcatatagacctcatcttggagtctctgcacaccggagagatggtaccgttggagtgtcatcatcacattgagtgcaatcactgtatagaggatatacccgacgagccaatcacgggggacccggcttggactgaagtcaagtttccttcaagtcctcaggagccctttctttacatcaggcaacaagatctgccggtcaaagacaaactcgagcctgtgcctcgcatgaacatcgtccgtcttgccggattgggtccggaggcgattagtgagctagcgcactactttgttgcattccgagttatccgggcgtcagagacggatgtcgaccctaacgatgttctctcgtggacctggctgagccgaattgatcctgacaaattggttgagtatatcgtgcatgtgttcgcttcactggaatggcatcatgtattaatgtcaggcgtgagtgtgagcgtcagagatgcattctttaagatgctagtgtctaaaagaatctcagagactccgctaagttcattctattatctggccaacctgttcgttgaccctcagactcgcgaagcactaatgagctctaaatacgggttcagcccccccgccgagacagtccccaacgcaaatgccgccgcagccgaaataagaagatgctgtgcgaacagtgcgccgtcgatcttagaatcagcccttcacagccgtgaggttgtttggatgccaggaacgaacaattatggagacgttgtcatctggtctcattacattagattacggttcagcgaagttaaactagttgacattacacgatatcagcagtggtggagacagtctgagcgagacccctacgatttggtcccggacatgcaggttcttgagagcgacctagatacgctgatgaaacggataccgaggctcatgcgcaaggcgagacgtccccctcttcaggtaattcgagaggacctggatgtcgcagtcatcaatgctgatcatcccgctcactctgtgcttcagaacaaatacaggaaattgattttcagagagccgaagattatcacgggagctgtgtacaagtacctctccctaaaatcagagttgacagagttcacctcagcaatggtgatcggagacggaactggaggtatcaccgccgccatgatggccgatgggatagatgtgtggtatcagacgctcgtcaactatgaccacgtgacacaacagggattatccgtacaagccccggcagcattggatcttctgcgcggggcaccctctggtaggctcttgaatccgggaagattcgcatcatttgggtctgacctaactgaccctcgatttacagcctactttgatcaatatcccccgttcaaggtggacactctatggtctgacgcagagggcgacttttgggacaagccttccaagttgaatcaatactttgagaacatcattgctttgagacatcggttcgtgaagacaaatggacagcttgtcgtgaaggtgtatctgactcaagacactgctaccacaattgaagcattcagaaagaagctgtccccatgcgccatcatcgtgtctctcttctcgacggaaggctccacagaatgcttcgtcctaagcaatctcatcgcaccagacacccctgtcgaccttgagatggtggagaatatccctaaactaacatcccttgttccccagaggacgacagtgaaatgctattcccgacgagtagcgtgcatcagtaaaaggtggggacttttcagatctccgagcatagcccttgaagtccaaccgttccttcactacatcacaaaggtcatctcagacaaaggaacacaactgagtctcatggcggtagctgacacaatgatcaacagttacaagaaggctatctcaccccgagtgttcgatctacaccggcatagggccgcactgggtttcgggaggagatccttgcatctcatctgggggatgatcatctcaccaatcgcttaccagcattttgagaatccggccaagttgatggatgtcctggacatgttgaccaataacatctcagctttcttatcgatatcgtcgtcaggatttgacctgtcatttagtgtcagtgcagaccgagatgtccggattgacagcaaacttgtcagactcccgctattcgaaggatcagacctaaaattcatgaaaaccatcatgtctaccctcggatctgtgttcaaccaggtcgagccttttaaggggatcgccataaacccttctaaactaatgactgtcaagaggacacaggagttacgttacaacaacctaatttacactaaggatgccatcctattccccaatgaagcggcaaaaaacactgccccgcttcgagccaacatggtataccccgtccggggagatctattcgcccctaccgatcgcataccaatcatgactctagtcagcgatgagacaacacctcagcactctcctccagaggatgaggca

SEQ ID NO: 13 - protein, 313 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSWGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 14 - protein, 321 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSWGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEDYKDDDDK

SEQ ID NO: 15 - protein, 158 aa, Artificial Sequence
  Artificial HPV16 E6 protein sequence. When all Xaa′s are cysteines, the sequence corresponds to the wildtype HPV16 E6 protein sequence.
  misc_feature (37)..(37): Xaa may present or absent. If present, Xaa can be any naturally occurring amino acid.
  misc_feature (40)..(40): Xaa may present or absent. If present, Xaa can be any naturally occurring amino acid..
  misc_feature (70)..(70): Xaa may present or absent. If present, Xaa can be any naturally occurring amino acid.
  misc_feature (73)..(73): Xaa may present or absent. If present, Xaa can be any naturally occurring amino acid.
  misc_feature (110)..(110): Xaa may present or absent. If present, Xaa can be any naturally occurring amino acid.
  misc_feature (113)..(113): Xaa may present or absent. If present, Xaa can be any naturally occurring amino acid.
  misc_feature (143)..(143): Xaa may present or absent. If present, Xaa can be any naturally occurring amino acid.
  misc_feature (146)..(146): Xaa may present or absent. If present, Xaa can be any naturally occurring amino acid.
MHQKRTAMFQDPQERPRKLPQLCTELQTTIHDIILEXVYXKQQLLRREVYDFAFRDLCIVYRDGNPYAVXDKXLKFYSKISEYRHYCYSLYGTTLEQQYNKPLCDLLIRXINXQKPLCPEEKQRHLDKKQRFHNIRGRWTGRXMSXCRSSRTRRETQL

SEQ ID NO: 16 - protein, 158 aa, Artificial Sequence
  Artificial HPV18 E6 protein sequence. When all Xaa′s are cysteines, the sequence corresponds to the wildtype HPV18 E6 protein sequence.
  misc_feature (32)..(32): Each X can be present or absent. If present, X can be any naturally occuring amino acid.
  misc_feature (35)..(35): Each X can be present or absent. If present, X can be any naturally occuring amino acid.
  misc_feature (65)..(65): Each X can be present or absent. If present, X can be any naturally occuring amino acid.
  misc_feature (68)..(68): Each X can be present or absent. If present, X can be any naturally occuring amino acid.
  misc_feature (105)..(105): Each X can be present or absent. If present, X can be any naturally occuring amino acid.
  misc_feature (108)..(108): Each X can be present or absent. If present, X can be any naturally occuring amino acid.
  misc_feature (138)..(138): Each X can be present or absent. If present, X can be any naturally occuring amino acid.
  misc_feature (141)..(141): Each X can be present or absent. If present, X can be any naturally occuring amino acid.
MARFEDPTRRPYKLPDLCTELNTSLQDIEITXVYXKTVLELTEVFEFAFKDLFVVYRDSIPHAAXHKXIDFYSRIRELRHYSDSVYGDTLEKLTNTGLYNLLIRXLRXQKPLNPAEKLRHLNEKRRFHNIAGHYRGQXHSXCNRARQERLQRRRETQV

SEQ ID NO: 17 - protein, 98 aa, Artificial Sequence
  Artificial HPV16 E7 protein sequence. When XaaXaaXaa is CYE and Xaa′s at positions 91 and 94 are cysteine, the sequence corresponds to the wildtype HPV16 E7 protein sequence.
  misc_feature (24)..(26): Xaa′s may be present or absent. If present, Xaa can be any naturally occurring amino acid.
  misc_feature (91)..(91): Xaa may be present or absent. If present, Xaa can be any naturally occurring amino acid.
  misc_feature (94)..(94): Xaa may be present or absent. If present, Xaa can be any naturally occurring amino acid.
MHGDTPTLHEYMLDLQPETTDLYXXXQLNDSSEEEDEIDGPAGQAEPDRAHYNIVTFCCKCDSTLRLCVQSTHVDIRTLEDLLMGTLGIVXPIXSQKP

SEQ ID NO: 18 - protein, 105 aa, Artificial Sequence
  Artificial HPV18 E7 protein sequence. When XaaXaaXaa is CHE and Xaa′s at positions 98 and 101 are cysteine, the sequence corresponds to the wildtype HPV18 E7 protein sequence.
  misc_feature (27)..(29): Xaa′s may be present or absent. If present, Xaa can be any naturally occurring amino acid
  misc_feature (98)..(98): Xaa may be present or absent. If present, Xaa can be any naturally occurring amino acid
  misc_feature (101)..(101): Xaa may be present or absent. If present, Xaa can be any naturally occurring amino acid
MHGPKATLQDIVLHLEPQNEIPVDLLXXXQLSDSEEENDEIDGVNHQHLPARRAEPQRHTMLCMCCKCEARIKLVVESSADDLRAFQQLFLNTLSFVXPWXASQQ

SEQ ID NO: 19 - protein, 341 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQLKL

SEQ ID NO: 20 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 21 - protein, 435 aa, Homo sapiens
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 22 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 23 - protein, 9 aa, Artificial Sequence
  tumour associated epitope
EVDPIGHLY

SEQ ID NO: 24 - protein, 9 aa, Artificial Sequence
  tumour associated epitope
FLWGPRALV

SEQ ID NO: 25 - protein, 9 aa, Artificial Sequence
  tumour associated epitope
KVAELVHFL

SEQ ID NO: 26 - protein, 9 aa, Artificial Sequence
  tumour associated epitope
TFPDLESEF

SEQ ID NO: 27 - protein, 9 aa, Artificial Sequence
  tumour associated epitope
VAELVHFLL

SEQ ID NO: 28 - protein, 10 aa, Artificial Sequence
  tumour associated epitope
REPVTKAEML

SEQ ID NO: 29 - protein, 9 aa, Artificial Sequence
  tumour associated epitope
AELVHFLLL

SEQ ID NO: 30 - protein, 9 aa, Artificial Sequence
  tumour associate epitope
WQYFFPVIF

SEQ ID NO: 31 - protein, 9 aa, Artificial Sequence
  tumour associated epitope
EGDCAPEEK

SEQ ID NO: 32 - protein, 16 aa, Artificial Sequence
  tumour associated epitope
KKLLTQHFVQENYLEY

SEQ ID NO: 33 - protein, 12 aa, Artificial Sequence
  tumour associated epitope
VIFSKASSSLQL

SEQ ID NO: 34 - protein, 15 aa, Artificial Sequence
  tumour associated epitope
VFGIELMEVDPIGHL

SEQ ID NO: 35 - protein, 15 aa, Artificial Sequence
  tumour associated epitope
GDNQIMPKAGLLIIV

SEQ ID NO: 36 - protein, 15 aa, Artificial Sequence
  tumour associated epitope
TSYVKVLHHMVKISG

SEQ ID NO: 37 - protein, 16 aa, Artificial Sequence
  tumour associated epitope
FLLLKYRAREPVTKAE